SEQUENCE LISTING (29 sequences)

SEQ ID NO: 1 - DNA, 1260 bp, SARS-COV-2
atgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaa

SEQ ID NO: 2 - DNA, 1329 bp, Artificial Sequence
  Synthetic Construct
atgcccatggggtctctgcaaccgctggccaccttgtacctgctggggatgctggtcgcttcctgcctcggatctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaa

SEQ ID NO: 3 - DNA, 2568 bp, Artificial Sequence
  Synthetic Construct
atgcccatggggtctctgcaaccgctggccaccttgtacctgctggggatgctggtcgcttcctgcctcggatctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggccatggcatcccctgctgcacctcgtgctgtttcctttgccgataacaatgatataacaaatacaaacctatctcgaggtagaggacgtaatccaaaaccacgagctgcaccaaataacactgtctcttggtacactgggcttacccaacacgggaaagtccctcttacctttccacctgggcagggtgtacctcttaatgccaattctacccctgcgcaaaatgctgggtattggcggagacaggacagaaaaattaataccgggaatggaattaagcaactggctcccaggtggtacttctactacactggaactggacccgaagcagcactcccattccgggctgttaaggatggcatcgtttgggtccatgaagatggcgccactgatgctccttcaacttttgggacgcggaaccctaacaatgattcagctattgttacacaattcgcgcccggtactaagcttcctaaaaacttccacattgaggggactggaggcaatagtcaatcatcttcaagagcctctagcttaagcagaaactcttccagatctagttcacaaggttcaagatcaggaaactctacccgcggcacttctccaggtccatctggaatcggagcagtaggaggtgatctactttaccttgatcttctgaacagactacaagcccttgagtctggcaaagtaaagcaatcgcagccaaaagtaatcactaagaaagatgctgctgctgctaaaaataagatgcgccacaagcgcacttccaccaaaagtttcaacatggtgcaagcttttggtcttcgcggaccaggagacctccagggaaactttggtgatcttcaattgaataaactcggcactgaggacccacgttggccccaaattgctgagcttgctcctacagccagtgcttttatgggtatgtcgcaatttaaacttacccatcagaacaatgatgatcatggcaaccctgtgtacttccttcggtacagtggagccattaaacttgacccaaagaatcccaactacaataagtggttggagcttcttgagcaaaatattgatgcctacaaaaccttccctaagaaggaaaagaaacaaaaggcaccaaaagaagaatcaacagaccaaatgtctgaacctccaaaggagcagcgtgtgcaaggtagcatcactcagcgcactcgcacccgtccaagtgttcagcctggtccaatgattgatgttaacactgattag

SEQ ID NO: 4 - DNA, 2568 bp, Artificial Sequence
  Synthetic Construct
atgcctatgggctctctgcagcccctggccaccctgtacctgctgggcatgctggtggccagctgcctgggaagcgacaacggcccccagaaccagagaaacgcccctagaatcacatttggcggccctagtgatagcaccggatctaatcaaaacggcgagagaagcggcgctcggtctaaacagagacggccacagggactgcctaacaacaccgccagctggttcaccgccctgacccagcacggcaaggaggaccttaagttccctcggggacagggcgtgccaatcaacaccaactctagtcccgacgaccagatcggctattatagaagagccacaagacgcatcagaggtggcgacggcaagatgaaggacctgagccctcgctggtacttttactacctggggaccggccctgaagccggcctgccttacggcgccaacaaggacggaatcatctgggtcgccaccgagggcgccctgaatacccctaaggaccacatcggcaccagaaaccctgctaataatgccgctatcgtgctgcagctgcctcagggcaccaccctgcctaagggcttctacgccgagggctcccggggaggttcccaggctagcagcagatcttccagccggagcagaaacagctccaggaacagcacacctggcagcagcagaggtacgagccctgcccggatggccggaaacggcggcgatgccgccctggccctgctgctgctggacagactgaaccagctcgagagcaagatgtctggcaagggccagcagcagcagggccagacagtgaccaagaaatccgccgctgaggccagcaaaaaacccagacagaaaagaaccgctacaaaggcctacaacgttacccaggcctttggcagacggggcccagagcagacccagggaaacttcggcgaccaggagctgatccggcagggcacggactacaagcactggcctcaaatcgcccagtttgccccttccgccagcgctttcttcggaatgagcagaatcggcatggaagtgacacctagtggcacctggctgacctacaccggtgccattaagctggatgacaaggaccccaacttcaaggatcaggtgatcctgctgaacaagcacattgatgcttacaagaccttcccacctaccgagccaaaaaaagataagaagaaaaaagccgatgagacacaagccctgccccagaggcagaagaagcaacaaaccgtcaccctgctgcctgctgccgacctggacgacttcagcaaacagctgcagcagagcatgagctctgctgatagcacccaggccatggcctctccagccgctcccagagctgtgtccttcgccgataataacgacatcacaaacaccaacctgagccggggcagaggcagaaaccctaaacctagagccgcccccaacaacaccgtgagctggtatacaggcctcacccagcatggcaaggtgcctctgacattcccccctgggcagggcgtgcccctgaacgccaacagcacccctgcccagaatgccggctactggcggaggcaggacagaaagatcaacactggtaacggcatcaagcagctggccccacggtggtatttctactacaccggcaccggcccggaagccgccctccccttcagagccgtgaaggacggcatcgtgtgggtgcacgaggacggcgccacagatgccccgtctacatttggcactcggaatcccaataacgacagcgccatcgtgacccagttcgcccctggcaccaagctgcctaagaactttcacatcgagggcacaggaggcaacagccagagcagcagccgggcttcgagcctgtctcggaatagctcccggtccagctctcagggcagccgcagtggaaattccacccggggcacatctcctggccccagcggcatcggcgctgtgggcggagacctgctctacctggacctgctgaacagactgcaggcacttgaaagcggcaaagttaagcaatctcaacctaaggtgatcaccaaaaaggacgccgccgccgctaagaacaagatgagacacaagagaacaagcacaaagagcttcaacatggtgcaagccttcggcctgcggggacctggcgacctgcagggcaacttcggcgacctgcagctgaacaagctgggcacagaggatcctcgatggccccagatcgccgaactagctccaaccgccagcgccttcatgggcatgagccagttcaagctgacacaccagaacaatgacgatcacggaaatcctgtgtacttcctgagatacagcggcgccatcaagctggatcctaagaaccccaactacaacaagtggctggaactgctggaacagaacatcgacgcctacaagaccttccccaagaaggaaaagaagcagaaggcccctaaagaggaaagcacagatcagatgagcgagcctcccaaggaacagagagtgcagggatctatcacccagagaacaagaacaagacccagcgtgcagcctggccctatgattgacgtgaacaccgactag

SEQ ID NO: 5 - protein, 419 aa, SARS-COV-2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 6 - protein, 442 aa, Artificial Sequence
  Synthetic Construct
MPMGSLQPLATLYLLGMLVASCLGSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 7 - protein, 855 aa, Artificial Sequence
  Synthetic Construct
MPMGSLQPLATLYLLGMLVASCLGSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQAMASPAAPRAVSFADNNDITNTNLSRGRGRNPKPRAAPNNTVSWYTGLTQHGKVPLTFPPGQGVPLNANSTPAQNAGYWRRQDRKINTGNGIKQLAPRWYFYYTGTGPEAALPFRAVKDGIVWVHEDGATDAPSTFGTRNPNNDSAIVTQFAPGTKLPKNFHIEGTGGNSQSSSRASSLSRNSSRSSSQGSRSGNSTRGTSPGPSGIGAVGGDLLYLDLLNRLQALESGKVKQSQPKVITKKDAAAAKNKMRHKRTSTKSFNMVQAFGLRGPGDLQGNFGDLQLNKLGTEDPRWPQIAELAPTASAFMGMSQFKLTHQNNDDHGNPVYFLRYSGAIKLDPKNPNYNKWLELLEQNIDAYKTFPKKEKKQKAPKEESTDQMSEPPKEQRVQGSITQRTRTRPSVQPGPMIDVNTD

SEQ ID NO: 8 - protein, 24 aa, Homo sapiens
MPMGSLQPLATLYLLGMLVASCLG

SEQ ID NO: 9 - protein, 413 aa, MERS
MASPAAPRAVSFADNNDITNTNLSRGRGRNPKPRAAPNNTVSWYTGLTQHGKVPLTFPPGQGVPLNANSTPAQNAGYWRRQDRKINTGNGIKQLAPRWYFYYTGTGPEAALPFRAVKDGIVWVHEDGATDAPSTFGTRNPNNDSAIVTQFAPGTKLPKNFHIEGTGGNSQSSSRASSLSRNSSRSSSQGSRSGNSTRGTSPGPSGIGAVGGDLLYLDLLNRLQALESGKVKQSQPKVITKKDAAAAKNKMRHKRTSTKSFNMVQAFGLRGPGDLQGNFGDLQLNKLGTEDPRWPQIAELAPTASAFMGMSQFKLTHQNNDDHGNPVYFLRYSGAIKLDPKNPNYNKWLELLEQNIDAYKTFPKKEKKQKAPKEESTDQMSEPPKEQRVQGSITQRTRTRPSVQPGPMIDVNTD

SEQ ID NO: 10 - protein, 422 aa, SARS-COV-1
MSDNGPQSNQRSAPRITFGGPTDSTDNNQNGGRNGARPKQRRPQGLPNNTASWFTALTQHGKEELRFPRGQGVPINTNSGPDDQIGYYRRATRRVRGGDGKMKELSPRWYFYYLGTGPEASLPYGANKEGIVWVATEGALNTPKDHIGTRNPNNNAATVLQLPQGTTLPKGFYAEGSRGGSQASSRSSSRSRGNSRNSTPGSSRGNSPARMASGGGETALALLLLDRLNQLESKVSGKGQQQQGQTVTKKSAAEASKKPRQKRTATKQYNVTQAFGRRGPEQTQGNFGDQDLIRQGTDYKHWPQIAQFAPSASAFFGMSRIGMEVTPSGTWLTYHGAIKLDDKDPQFKDNVILLNKHIDAYKTFPPTEPKKDKKKKTDEAQPLPQRQKKQPTVTLLPAADMDDFSRQLQNSMSGASADSTQA

SEQ ID NO: 11 - DNA, 26 bp, Artificial Sequence
  Synthetic Construct
ggcgcgccattggccaccgcggccgc

SEQ ID NO: 12 - DNA, 10 bp, Artificial Sequence
  Synthetic Construct
attggccacc

SEQ ID NO: 13 - protein, 497 aa, Influenza A Virus
ASQGTKRSYEQMETGGERQNATEIRASVGRMVGGIGRFYIQMCTELKLSDYEGRLIQNSITIERMVLSAFDERRNKYLEEHPSAGKDPKKTGGPIYRRRDGKWVRELILYDKEEIRRIWRQANNGEDATAGLTHLMIWHSNLNDATYQRTRALVRTGMDPRMCSLMQGSTLPRRSGAAGAAVKGVGTMVMELIRMIKRGINDRNFWRGENGRRTRIAYERMCNILKGKFQTAAQRAMMDQVRESRNPGNAEIEDLIFLARSALILRGSVAHKSCLPACVYGLAVASGYDFEREGYSLVGIDPFRLLQNSQVFSLIRPNENPAHKSQLVWMACHSAAFEDLRVSSFIRGTRVVPRGQLSTRGVQIASNENMETMDSSTLELRSRYWAIRTRSGGTTNQQRASAGQISVQPTFSVQRNLPFERATIMAAFTGNTEGRTSDMRTEIIRMMESAKPEDVSFQGRGVFELSDEKATNPIVPSFDMSNEGSYFFGDNAEEYDN

SEQ ID NO: 14 - protein, 560 aa, Influenza B Virus
MSNMDIDGINTGTIDKTPEEITPGTSGTTRPIIRPATLAPPSNKRTRNPSPERATTSSEDDVGRKTQKKQTPTEIKKSVYNMVVKLGEFYNQMMVKAGLNDDMERNLIQNAHAVERILLAATDDKKTEFQKKKNARDVKEGKEEIDHNKTGGTFYKMVRDDKTIYFSPIRITFLKEEVKTMYKTTMGSDGFSGLNHIMIGHSQMNDVCFQRSKALKRVGLDPSLISTFAGSTIPRRSGATGVAIKGGGTLVAEAIRFIGRAMADRGLLRDIKAKTAYEKILLNLKNKCSAPQQKALVDQVIGSRNPGIADIEDLTLLARSMVVVRPSVASKVVLPISIYAKIPQLGFNVEEYSMVGYEAMALYNMATPVSILRMGDDAKDKSQLFFMSCFGAAYEDLRVLSALTGTEFKPRSALKCKGFHVPAKEQVEGMGAALMSIKLQFWAPMTRSGGNEVGGDGGSGQISCSPVFAVERPIALSKQAVRRMLSMNIEGRDADVKGNLLKMMNDSMAKKTSGNAFIGKKMFQISDKNKTNPVEIPIKQTIPNFFFGRDTAEDYDDLDY

SEQ ID NO: 15 - DNA, 3246 bp, Artificial Sequence
  Synthetic Construct
atgcctatgggcagcctgcagccactggctacactgtacctgctgggcatgctggtggcctcttgtctgggcgccagccaaggcactaagagaagctacgagcagatggaaaccggaggcgaacggcagaacgccacagagatcagagcctctgtgggccgtatggtcggcggcatcggcagattctacatccagatgtgcaccgaactgaagctgagcgactacgagggccgcctgatccagaacagcatcacaatcgagagaatggtgctgtccgcctttgacgagcggagaaacaaatacctggaagagcaccctagcgccggaaaagatcctaagaaaaccggcggacctatctacagaagaagagatggtaagtgggtgagagagctgattctgtacgataaggaagagattcgaagaatctggagacaggccaacaacggcgaggatgccaccgcaggcctgacacacctgatgatctggcacagcaacctgaacgatgcgacctaccagcgcacgcgggccctggtcagaaccggcatggatcctcggatgtgtagcctgatgcagggcagcacactgccaagacggagtggggccgccggcgctgcagtgaagggcgtcggaaccatggtgatggagctgatccggatgataaagcggggcatcaacgacagaaacttctggcgaggcgagaacggccgaagaacccggatcgcctacgagagaatgtgcaacatcctgaaaggaaaattccagaccgccgcccagcgggccatgatggaccaggtgcgcgagagcagaaaccccggcaatgccgagatcgaggacctgatcttcctggccagaagcgccctcattcttagaggctctgtggcccacaagagctgtctgcctgcctgtgtgtacggcctggcagtggcctcaggctacgacttcgagcgggaaggatacagtctggtgggcatcgaccctttcagactcctgcagaatagccaggtgtttagcctgatcagaccaaacgaaaaccccgcccataagagccagctggtgtggatggcctgccacagcgccgcctttgaggatctgagagtgagctcttttatcagaggcacccgggtggttccacgaggtcaactgtctacaagaggtgtgcagatcgccagcaacgagaacatggagaccatggatagcagcaccctggaactgagatccagatactgggccatcaggacacggagcggcggcaccaccaatcagcagcgcgccagcgccggccagatctctgtccagcctacgtttagcgtgcagcggaatttgcccttcgaacgcgccacaatcatggctgctttcaccggcaatacagagggcagaaccagcgatatgagaacagaaattatccgtatgatggagtccgcaaaacctgaggacgtgtccttccaaggcagaggcgtgttcgagctgagcgacgagaaggccaccaaccctatcgtgcctagcttcgatatgtctaatgagggcagctactttttcggagataacgccgaagagtacgacaacatgtctaatatggatatcgacggcattaacaccggcaccatcgacaaaacccctgaggagatcacccctggcaccagcggcacaacccggcccatcatccgccccgctacactggctccacctagcaacaagcggaccagaaatccctcgccagaaagagccacaacctccagcgaggacgacgtgggacggaagacacaaaagaagcagacccctacagagatcaagaagtctgtttacaacatggtggtgaaactgggcgagttctacaaccagatgatggtgaaggccggcctgaacgacgatatggaaagaaatctgatccagaacgcccacgccgtggagcggattctgctggccgccaccgatgataagaagaccgaattccagaaaaagaaaaacgccagagacgtgaaggaaggcaaggaagagatcgaccacaacaagacaggcggcacattctacaagatggtccgggacgacaagaccatctacttcagccctatccggataacattcctgaaagaagaagtgaagaccatgtacaaaaccacaatgggctctgacggcttcagcggcctgaatcacatcatgatcggccactctcaaatgaacgatgtgtgcttccagagaagcaaggctctgaagcgcgtgggcctggatcctagcctgatctctaccttcgccggcagcaccatccccagaagatcgggcgctaccggcgtggctatcaagggaggaggcacactggtggctgaagccatcagattcatcggaagagccatggccgacagaggactcctgagagatatcaaagccaaaaccgcctacgaaaaaatcctgctgaacctgaagaacaagtgcagcgcgcctcaacagaaggccctggtggaccaggttatcggctctagaaaccctggaatcgccgatatcgaggacctgacactgctggccagatctatggtggtggtgagaccctccgtggccagcaaggtggtgctgcctatcagcatctacgccaagatccctcagctgggatttaacgtggaagaatacagcatggttggttatgaggccatggccctgtacaacatggccacacctgtgtccatcctgagaatgggcgacgatgccaaagacaagagccagctgttcttcatgagctgcttcggcgctgcctatgaggacctgagagtgctgtccgctcttacaggaacagagttcaagcctaggagcgcactgaagtgcaagggcttccacgtgcccgccaaggaacaggtggaaggcatgggagctgctctgatgtccatcaagctgcaattttgggctcctatgacccggagcggcggaaatgaggtgggtggcgacggaggcagcggacagatttcttgcagccccgtatttgccgtggagagaccaatcgccctgtccaagcaggccgtgagaagaatgctgagcatgaacatcgagggccgggacgccgacgtgaagggcaacctgttgaagatgatgaacgacagcatggccaagaagaccagtggcaatgccttcatcggcaagaagatgttccagatctccgacaagaacaagaccaaccccgtggaaatccccatcaagcagacaatccctaacttcttcttcggcagagacaccgccgaagactatgacgacctggactactga

SEQ ID NO: 16 - protein, 1081 aa, Artificial Sequence
  Synthetic Construct
MPMGSLQPLATLYLLGMLVASCLGASQGTKRSYEQMETGGERQNATEIRASVGRMVGGIGRFYIQMCTELKLSDYEGRLIQNSITIERMVLSAFDERRNKYLEEHPSAGKDPKKTGGPIYRRRDGKWVRELILYDKEEIRRIWRQANNGEDATAGLTHLMIWHSNLNDATYQRTRALVRTGMDPRMCSLMQGSTLPRRSGAAGAAVKGVGTMVMELIRMIKRGINDRNFWRGENGRRTRIAYERMCNILKGKFQTAAQRAMMDQVRESRNPGNAEIEDLIFLARSALILRGSVAHKSCLPACVYGLAVASGYDFEREGYSLVGIDPFRLLQNSQVFSLIRPNENPAHKSQLVWMACHSAAFEDLRVSSFIRGTRVVPRGQLSTRGVQIASNENMETMDSSTLELRSRYWAIRTRSGGTTNQQRASAGQISVQPTFSVQRNLPFERATIMAAFTGNTEGRTSDMRTEIIRMMESAKPEDVSFQGRGVFELSDEKATNPIVPSFDMSNEGSYFFGDNAEEYDNMSNMDIDGINTGTIDKTPEEITPGTSGTTRPIIRPATLAPPSNKRTRNPSPERATTSSEDDVGRKTQKKQTPTEIKKSVYNMVVKLGEFYNQMMVKAGLNDDMERNLIQNAHAVERILLAATDDKKTEFQKKKNARDVKEGKEEIDHNKTGGTFYKMVRDDKTIYFSPIRITFLKEEVKTMYKTTMGSDGFSGLNHIMIGHSQMNDVCFQRSKALKRVGLDPSLISTFAGSTIPRRSGATGVAIKGGGTLVAEAIRFIGRAMADRGLLRDIKAKTAYEKILLNLKNKCSAPQQKALVDQVIGSRNPGIADIEDLTLLARSMVVVRPSVASKVVLPISIYAKIPQLGFNVEEYSMVGYEAMALYNMATPVSILRMGDDAKDKSQLFFMSCFGAAYEDLRVLSALTGTEFKPRSALKCKGFHVPAKEQVEGMGAALMSIKLQFWAPMTRSGGNEVGGDGGSGQISCSPVFAVERPIALSKQAVRRMLSMNIEGRDADVKGNLLKMMNDSMAKKTSGNAFIGKKMFQISDKNKTNPVEIPIKQTIPNFFFGRDTAEDYDDLDY

SEQ ID NO: 17 - protein, 497 aa, Influenza A Virus
ASQGTKRSYEQMETDGERQNANEIRASVGKMIGGIGRFYIQMCTELKLSDYEGRLIQNSLTIERMVLSAFDERRNKYLEEHPSAGKDPKKTGGPIYKRVDGKWMRELVLYDKEEIRRIWRQANNGDDATAGLTHMMIWHSNLNDTTYQRTRALVRTGMDPRMCSLMQGSTLPRRSGAAGAAVKGVGTMVMELIRMIKRGINDRNFWRGENGRKTRNAYERMCNILKGKFQTAAQRAMMDQVRESRNPGNAEIEDLIFLARSALILRGSVAHKSCLPACVYGPAVASGYDFEKEGYSLVGIDPFKLLQNSQVYSLIRPNENPAHKSQLVWMACNSAAFEDLRVSSFIRGTKVIPRGKLSTRGVQIASNENMDTMGSSTLELRSRYWAIRTRSGGNTNQQRASAGQISVQPTFSVQRNLPFDKPTIMAAFTGNAEGRTSDMRAEIIRMMEGAKPEEVSFQGRGVFELSDEKATNPIVPSFDMSNEGSYFFGDNAEEYDN

SEQ ID NO: 18 - protein, 738 aa, Zaire ebolavirus
DSRPQKIWMAPSLTESDMDYHKILTAGLSVQQGIVRQRVIPVYQVNNLEEICQLIIQAFEAGVDFQESADSFLLMLCLHHAYQGDYKLFLESGAVKYLEGHGFRFEVKKRDGVKRLEELLPAVSSGKNIKRTLAAMPEEETTEANAGQFLSFASLFLPKLVVGEKACLEKVQRQIQVHAEQGLIQYPTAWQSVGHMMVIFRLMRTNFLIKFLLIHQGMHMVAGHDANDAVISNSVAQARFSGLLIVKTVLDHILQKTERGVRLHPLARTAKVKNEVNSFKAALSSLAKHGEYAPFARLLNLSGVNNLEHGLFPQLSAIALGVATAHGSTLAGVNVGEQYQQLREAATEAEKQLQQYAESRELDHLGLDDQEKKILMNFHQKKNEISFQQTNAMVTLRKERLAKLTEAITAASLPKTSGHYDDDDDIPFPGPINDDDNPGHQDDDPTDSQDTTIPDVVVDPDDGSYGEYQSYSENGMNAPDDLVLFDLDEDDEDTKPVPNRSTKGGQQKNSQKGQHIEGRQTQFRPIQNVPGPHRTIHHASAPLTDNDRRNEPSGSTSPRMLTPINEEADPLDDADDETSSLPPLESDDEEQDRDGTSNRTPTVAPPAPVYRDHSEKKELPQDEQQDQDHTQEARNQDSDNTQSEHSLEEMYRHILRSQGPFDAVLYYHMMKDEPVVFSTSDGKEYTYPDSLEEEYPPWLTEKEAMNEENRFVTLDGQQFYWPVMNHKNKFMAILQHHQ

SEQ ID NO: 19 - protein, 336 aa, Sudan ebolavirus
AKLTEAITTASKIKVGDRYPDDNDIPFPGPIYDDTHPNPSDDNPDDSRDTTIPGGVVDPYDDESNNYPDYEDSAEGTTGDLDLFNLDDDDDDSRPGPPDRGQNKERAARTYGLQDPTLDGAKKVPELTPGSHQPGNLHITKSGSNTNQPQGNMSSTLHSMTPIQEESEPDDQKDNDDESLTSLDSEGDEDGESISEENTPTVAPPAPVYKDTGVDTNQQNGPSSTVDSQGSESEALPINSKKSSALEETYYHLLKTQGPFEAINYYHLMSDEPIAFSTESGKEYIFPDSLEEAYPPWLSEKEALEKENRYLVIDGQQFLWPVMSLRDKFLAVLQHD

SEQ ID NO: 20 - protein, 337 aa, Bundibugyo ebolavirus
AKLTEAITSTSILKTGRRYDDDNDIPFPGPINDNENSGQNDDDPTDSQDTTIPDVIIDPNDGGYNNYSDYANDAASAPDDLVLFDLEDEDDADNPAQNTPEKNDRPATTKLRNGQDQDGNQGETASPRVAPNQYRDKPMPQVQDRSENHDQTLQTQSRVLTPISEEADPSDHNDGDNESIPPLESDDEGSTDTTAAETKPATAPPAPVYRSISVDDSVPSENIPAQSNQTNNEDNVRNNAQSEQSIAEMYQHILKTQGPFDAILYYHMMKEEPIIFSTSDGKEYTYPDSLEDEYPPWLSEKEAMNEDNRFITMDGQQFYWPVMNHRNKFMAILQHHR

SEQ ID NO: 21 - protein, 169 aa, Tai Forest ebolavirus
LVLFDLEDGDEDDHRPSSSSENNNKHSLTGTDSNKTSNWNRNPTNMPKKDSTQNNDNPAQRAQEYARDNIQDTPTPHRALTPISEETGSNGHNEDDIDSIPPLESDEENNTETTITTTKNTTAPPAPVYRSNSEKEPLPQEKSQKQPNQVSGSENTDNKPHSEQSVEEM

SEQ ID NO: 22 - protein, 1604 aa, Artificial Sequence
  Synthetic Construct
MPMGSLQPLATLYLLGMLVASCLGDSRPQKIWMAPSLTESDMDYHKILTAGLSVQQGIVRQRVIPVYQVNNLEEICQLIIQAFEAGVDFQESADSFLLMLCLHHAYQGDYKLFLESGAVKYLEGHGFRFEVKKRDGVKRLEELLPAVSSGKNIKRTLAAMPEEETTEANAGQFLSFASLFLPKLVVGEKACLEKVQRQIQVHAEQGLIQYPTAWQSVGHMMVIFRLMRTNFLIKFLLIHQGMHMVAGHDANDAVISNSVAQARFSGLLIVKTVLDHILQKTERGVRLHPLARTAKVKNEVNSFKAALSSLAKHGEYAPFARLLNLSGVNNLEHGLFPQLSAIALGVATAHGSTLAGVNVGEQYQQLREAATEAEKQLQQYAESRELDHLGLDDQEKKILMNFHQKKNEISFQQTNAMVTLRKERLAKLTEAITAASLPKTSGHYDDDDDIPFPGPINDDDNPGHQDDDPTDSQDTTIPDVVVDPDDGSYGEYQSYSENGMNAPDDLVLFDLDEDDEDTKPVPNRSTKGGQQKNSQKGQHIEGRQTQFRPIQNVPGPHRTIHHASAPLTDNDRRNEPSGSTSPRMLTPINEEADPLDDADDETSSLPPLESDDEEQDRDGTSNRTPTVAPPAPVYRDHSEKKELPQDEQQDQDHTQEARNQDSDNTQSEHSLEEMYRHILRSQGPFDAVLYYHMMKDEPVVFSTSDGKEYTYPDSLEEEYPPWLTEKEAMNEENRFVTLDGQQFYWPVMNHKNKFMAILQHHQAKLTEAITTASKIKVGDRYPDDNDIPFPGPIYDDTHPNPSDDNPDDSRDTTIPGGVVDPYDDESNNYPDYEDSAEGTTGDLDLFNLDDDDDDSRPGPPDRGQNKERAARTYGLQDPTLDGAKKVPELTPGSHQPGNLHITKSGSNTNQPQGNMSSTLHSMTPIQEESEPDDQKDNDDESLTSLDSEGDEDGESISEENTPTVAPPAPVYKDTGVDTNQQNGPSSTVDSQGSESEALPINSKKSSALEETYYHLLKTQGPFEAINYYHLMSDEPIAFSTESGKEYIFPDSLEEAYPPWLSEKEALEKENRYLVIDGQQFLWPVMSLRDKFLAVLQHDAKLTEAITSTSILKTGRRYDDDNDIPFPGPINDNENSGQNDDDPTDSQDTTIPDVIIDPNDGGYNNYSDYANDAASAPDDLVLFDLEDEDDADNPAQNTPEKNDRPATTKLRNGQDQDGNQGETASPRVAPNQYRDKPMPQVQDRSENHDQTLQTQSRVLTPISEEADPSDHNDGDNESIPPLESDDEGSTDTTAAETKPATAPPAPVYRSISVDDSVPSENIPAQSNQTNNEDNVRNNAQSEQSIAEMYQHILKTQGPFDAILYYHMMKEEPIIFSTSDGKEYTYPDSLEDEYPPWLSEKEAMNEDNRFITMDGQQFYWPVMNHRNKFMAILQHHRLVLFDLEDGDEDDHRPSSSSENNNKHSLTGTDSNKTSNWNRNPTNMPKKDSTQNNDNPAQRAQEYARDNIQDTPTPHRALTPISEETGSNGHNEDDIDSIPPLESDEENNTETTITTTKNTTAPPAPVYRSNSEKEPLPQEKSQKQPNQVSGSENTDNKPHSEQSVEEM

SEQ ID NO: 23 - DNA, 4815 bp, Artificial Sequence
  Synthetic Construct
atgcccatggggtctctgcaaccgctggccaccttgtacctgctggggatgctggtcgcttcctgcctcggagattctcgtcctcagaaaatctggatggcgccgagtctcactgaatctgacatggattaccacaagatcttgacagcaggtctgtccgttcaacaggggattgttcggcaaagagtcatcccagtgtatcaagtaaacaatcttgaagaaatttgccaacttatcatacaggcctttgaagcaggtgttgattttcaagagagtgcggacagtttccttctcatgctttgtcttcatcatgcgtaccagggagattacaaacttttcttggaaagtggcgcagtcaagtatttggaagggcacgggttccgttttgaagtcaagaagcgtgatggagtgaagcgccttgaggaattgctgccagcagtatctagtggaaaaaacattaagagaacacttgctgccatgccggaagaggagacaactgaagctaatgccggtcagtttctctcctttgcaagtctattccttccgaaattggtagtaggagaaaaggcttgccttgagaaggttcaaaggcaaattcaagtacatgcagagcaaggactgatacaatatccaacagcttggcaatcagtaggacacatgatggtgattttccgtttgatgcgaacaaattttctgatcaaatttctcctaatacaccaagggatgcacatggttgccgggcatgatgccaacgatgctgtgatttcaaattcagtggctcaagctcgtttttcaggcttattgattgtcaaaacagtacttgatcatatcctacaaaagacagaacgaggagttcgtctccatcctcttgcaaggaccgccaaggtaaaaaatgaggtgaactcctttaaggctgcactcagctccctggccaagcatggagagtatgctcctttcgcccgacttttgaacctttctggagtaaataatcttgagcatggtcttttccctcaactatcggcaattgcactcggagtcgccacagcacacgggagtaccctcgcaggagtaaatgttggagaacagtatcaacaactcagagaggctgccactgaggctgagaagcaactccaacaatatgcagagtctcgcgaacttgaccatcttggacttgatgatcaggaaaagaaaattcttatgaacttccatcagaaaaagaacgaaatcagcttccagcaaacaaacgctatggtaactctaagaaaagagcgcctggccaagctgacagaagctatcactgctgcgtcactgcccaaaacaagtggacattacgatgatgatgacgacattccctttccaggacccatcaatgatgacgacaatcctggccatcaagatgatgatccgactgactcacaggatacgaccattcccgatgtggtggttgatcctgatgatggaagctacggcgaataccagagttactcggaaaacggcatgaatgcaccagatgacttggtcctattcgatctagacgaggacgacgaggacactaagccagtgcctaatagatcgaccaagggtggacaacagaagaacagtcaaaagggccagcatatagagggcagacagacacaattcaggccaattcaaaatgtcccaggccctcacagaacaatccaccacgccagtgcgccactcacggacaatgacagaagaaatgaaccctccggctcaaccagccctcgcatgctgacaccaattaacgaagaggcagacccactggacgatgccgacgacgagacgtctagccttccgcccttggagtcagatgatgaagagcaggacagggacggaacttccaaccgcacacccactgtcgccccaccggctcccgtatacagagatcactctgaaaagaaagaactcccgcaagacgagcaacaagatcaggaccacactcaagaggccaggaaccaggacagtgacaacacccagtcagaacactcccttgaggagatgtatcgccacattctaagatcacaggggccatttgatgctgttttgtattatcatatgatgaaggatgagcctgtagttttcagtaccagtgatggcaaagagtacacgtatccagactcccttgaagaggaatatccaccatggctcactgaaaaagaggctatgaatgaagagaatagatttgttacattggatggtcaacaattttattggccggtgatgaatcacaagaataaattcatggcaatcctgcaacatcatcaggctaaattgaccgaagccatcacgactgcatcgaagatcaaggttggagaccgttatcctgatgacaatgatattccatttcccgggccgatctatgatgacactcaccccaatccctctgatgacaatcctgatgattcacgtgatacaactattccaggtggtgttgttgacccgtatgatgatgagagtaataattatcctgactacgaggattcggctgaaggcaccacaggagatcttgatctcttcaatttggacgacgacgatgatgacagccgaccaggaccaccagacagggggcagaacaaggagagggcggcccggacatatggcctccaagatccgaccttggacggagcgaaaaaggtgccggagttgaccccaggttcccatcaaccaggcaacctccacatcaccaagtcgggttcaaacaccaaccaaccacaaggcaatatgtcatctactctccatagtatgacccctatacaggaagaatcagagcccgatgatcaaaaagataatgatgacgagagtctcacatcccttgactctgaaggtgacgaagatggtgagagcatctctgaggagaacaccccaactgtagctccaccagcaccagtctacaaagacactggagtagacactaatcagcagaatggaccaagcagtactgtagatagtcaaggttctgaaagtgaagctctcccaatcaactctaaaaagagttccgcactagaagaaacatattatcatctcctaaaaacacagggtccatttgaggcaatcaattattatcacctaatgagtgatgaacccattgcttttagcactgaaagtggcaaggaatatatctttccagactcccttgaagaagcctacccgccgtggttgagtgagaaggaggccttagagaaggaaaatcgttatctggtcattgatggccagcaattcctctggccggtaatgagcctacgggacaagttccttgccgttcttcaacatgacgccaaattgaccgaagctattacttccacctctatcctcaaaacaggaaggcggtatgatgatgacaatgacataccctttccagggccaatcaatgataacgagaactctggtcagaacgatgacgatccaacagactcccaggataccacaatcccggatgtaataatcgatccaaacgatggtgggtataataattacagcgattatgcaaatgatgctgcaagtgctcctgatgacctagttctttttgaccttgaggacgaggatgatgctgataacccggctcaaaacacgccagaaaaaaatgatagaccagcaacaacaaagctgagaaatggacaggaccaggatggaaaccaaggcgaaactgcatccccacgggtagcccccaaccaatacagagacaagccaatgccacaagtacaggacagatccgaaaatcatgaccaaacccttcaaacacagtccagggttttgactcctatcagcgaggaagcagaccccagcgaccacaacgatggtgacaatgaaagcattcctcccctggaatcagacgacgagggtagcactgatactactgcagcagaaacaaagcctgccactgcacctcccgctcccgtctaccgaagtatctccgtagatgattctgtcccctcagagaacattcccgcacagtccaatcaaacgaacaatgaggacaatgtcaggaacaatgctcagtcggagcaatccattgcagaaatgtatcaacatatcttgaaaacacaaggaccttttgatgccatcctttactaccatatgatgaaagaagagcccatcattttcagcactagtgatgggaaggagtatacatatccagactctcttgaagatgagtatccaccctggctcagcgagaaggaagccatgaacgaagacaatagattcataaccatggatggtcagcagttttactggcctgtgatgaatcatagaaataaattcatggcaatcctccagcatcacaggcttgttctttttgaccttgaagatggtgacgaggatgatcaccgaccgtcaagttcatcagagaacaacaacaaacacagtcttacaggaactgacagtaacaaaacaagtaactggaatcgaaacccgactaatatgccaaagaaagactccacacaaaacaatgacaatcctgcacagcgggctcaagaatacgccagggataacatccaggatacaccaacaccccatcgagctctaactcccatcagcgaagaaaccggctccaatggtcacaatgaagatgacattgatagcatccctcctttggaatcagacgaagaaaacaacactgagacaaccattaccaccacaaaaaataccactgctccaccagcacctgtttatcggagtaattcagaaaaggagcccctcccgcaagaaaaatcccagaagcaaccaaaccaagtgagtggtagtgagaataccgacaataaacctcactcagagcaatcagtggaagaaatgtaa

SEQ ID NO: 24 - DNA, 744 bp, Artificial Sequence
  Synthetic Construct
atgcccatggggtctctgcaaccgctggccaccttgtacctgctggggatgctggtcgcttcctgcctcggaagagtccaaccaacagaatctattgttagatttcctaatattacaaacttgtgcccttttgatgaagtttttaacgccaccagatttgcatctgtttatgcttggaacaggaagagaatcagcaactgtgttgctgattattctgtcctatataatctcgcaccatttttcacttttaagtgttatggagtgtctcctactaaattaaatgatctctgctttactaatgtctatgcagattcatttgtaattagaggtgatgaagtcagacaaatcgctccagggcaaactggaaacattgctgattataattataaattaccagatgattttacaggctgcgttatagcttggaattctaacaagcttgattctaaggttagtggtaattataattacctgtatagattgtttaggaagtctaatctcaaaccttttgagagagatatttcaactgaaatctatcaggccggtaacaaaccttgtaatggtgttgcaggttttaattgttactttcctttacgatcatatagtttccgacccacttatggtgttggtcaccaaccatacagagtagtagtactttcttttgaacttctacatgcaccagcaactgtttgtggacctaaaaagtctactaatttggttaaaaacaaatgtgtcaatttctaa

SEQ ID NO: 25 - protein, 247 aa, Artificial Sequence
  Synthetic Construct
MPMGSLQPLATLYLLGMLVASCLGRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNF

SEQ ID NO: 26 - DNA, 4056 bp, Artificial Sequence
  Synthetic Construct
atgcccatggggtctctgcaaccgctggccaccttgtacctgctggggatgctggtcgcttcctgcctcggaagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattaccggtatagattgtttaggaagtctaatctcaaaccttttgagagagatatttcaactgaaatctatcaggccggtagcaaaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttctcaggtggtggcggttcaggcggaggtggctctggcggtggcggatcgatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaaggccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtatgggaacttctcctgctagaatggctggcaatggctgtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggcttatgaaactcaagccttaccgcagagacagaagaaacagcaaactgtgactcttcttcctgctgcagatttggatgatttctccaaacaattgcaacaatccatgagcagtgctgactcaactcaggccatggcatcccctgctgcacctcgtgctgtttcctttgccgataacaatgatataacaaatacaaacctatctcgaggtagaggacgtaatccaaaaccacgagctgcaccaaataacactgtctcttggtacactgggcttacccaacacgggaaagtccctcttacctttccacctgggcagggtgtacctcttaatgccaattctacccctgcgcaaaatgctgggtattggcggagacaggacagaaaaattaataccgggaatggaattaagcaactggctcccaggtggtacttctactacactggaactggacccgaagcagcactcccattccgggctgttaaggatggcatcgtttgggtccatgaagatggcgccactgatgctccttcaacttttgggacgcggaaccctaacaatgattcagctattgttacacaattcgcgcccggtactaagcttcctaaaaacttccacattgaggggactggaggcaatagtcaatcatcttcaagagcctctagcttaagcagaaactcttccagatctagttcacaaggttcaagatcaggaaactctacccgcggcacttctccaggtccatctggaatcggagcagtaggaggtgatctactttaccttgatcttctgaacagactacaagcccttgagtctggcaaagtaaagcaatcgcagccaaaagtaatcactaagaaagatgctgctgctgctaaaaataagatgcgccacaagcgcacttccaccaaaagtttcaacatggtgcaagcttttggtcttcgcggaccaggagacctccagggaaactttggtgatcttcaattgaataaactcggcactgaggacccacgttggccccaaattgctgagcttgctcctacagccagtgcttttatgggtatgtcgcaatttaaacttacccatcagaacaatgatgatcatggcaaccctgtgtacttccttcggtacagtggagccattaaacttgacccaaagaatcccaactacaataagtggttggagcttcttgagcaaaatattgatgcctacaaaaccttccctaagaaggaaaagaaacaaaaggcaccaaaagaagaatcaacagaccaaatgtctgaacctccaaaggagcagcgtgtgcaaggtagcatcactcagcgcactcgcacccgtccaagtgttcagcctggtccaatgattgatgttaacactgattctggtggcggtggctcgggcggaggtgggtcgggtggcggcggatcagaagcaaaaccttctggctcagttgtggaacaggctgaaggtgttgaatgtgatttttcacctcttctgtctggcacacctcctcaggtttataatttcaagcgtttggtttttaccaattgcaattataatcttaccaaattgctttcacttttttctgtgaatgattttacttgtagtcaaatatctccagcagcaattgctagcaactgttattcttcactgattttggattacttttcatacccacttagtatgaaatccgatctcagtgttagttctgctggtccaatatcccagtttaattataaacagtccttttctaatcccacatgtttgattttagcgactgttcctcataaccttactactattactaagcctcttaagtacagctatattaacaagtgctctcgtcttctttctgatgatcgtactgaagtacctcagttagtgaacgctaatcaatactcaccctgtgtatccattgtcccatccactgtgtgggaagacggtgattattataggaaacaactatctccacttgaaggtggtggctggcttgttgctagtggctcaactgttgccatgactgagcaattacagatgggctttggtattacagttcaatatggtacagacaccaatagtgtttgccccaagcttgaatttgctaatgacacaaaaattgcctctcaattaggcaattgcgtggaatattag

SEQ ID NO: 27 - protein, 1351 aa, Artificial Sequence
  Synthetic Construct
MPMGSLQPLATLYLLGMLVASCLGRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFSGGGGSGGGGSGGGGSMSDNGPQNQRNAPRITFGGPSDSTGSNQNGERSGARSKQRRPQGLPNNTASWFTALTQHGKEGLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSMGTSPARMAGNGCDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKAYETQALPQRQKKQQTVTLLPAADLDDFSKQLQQSMSSADSTQAMASPAAPRAVSFADNNDITNTNLSRGRGRNPKPRAAPNNTVSWYTGLTQHGKVPLTFPPGQGVPLNANSTPAQNAGYWRRQDRKINTGNGIKQLAPRWYFYYTGTGPEAALPFRAVKDGIVWVHEDGATDAPSTFGTRNPNNDSAIVTQFAPGTKLPKNFHIEGTGGNSQSSSRASSLSRNSSRSSSQGSRSGNSTRGTSPGPSGIGAVGGDLLYLDLLNRLQALESGKVKQSQPKVITKKDAAAAKNKMRHKRTSTKSFNMVQAFGLRGPGDLQGNFGDLQLNKLGTEDPRWPQIAELAPTASAFMGMSQFKLTHQNNDDHGNPVYFLRYSGAIKLDPKNPNYNKWLELLEQNIDAYKTFPKKEKKQKAPKEESTDQMSEPPKEQRVQGSITQRTRTRPSVQPGPMIDVNTDSGGGGSGGGGSGGGGSEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNCVEY

SEQ ID NO: 28 - DNA, 4098 bp, Artificial Sequence
  Synthetic Construct
atgcctatgggcagcctgcagccactggctacactgtacctgctgggcatgctggtggcctcttgtctgggcataggagagtgtccaaagtatgtcaggagtgcaaaattaaggatggttacaggactaaggaacatcccatccattcaatccagaggtttgtttggagccattgccggtttcattgaaggggggtggactggaatggtagatgggtggtatggttatcatcatcagaatgagcaaggatctggctatgctgcagatcaaaaaagtacacaaaatgccattaacgggattacaaacaaggtgaattctgtaattgagaaaatgaacactcaattcacagctgtgggcaaagaattcaacaaattggaaagaaggatggaaaacttaaataaaaaagttgatgatgggtttctagacatttggacatataatgcagaattgttggttctactggaaaatgaaaggactttggatttccatgactccgccagccaaggcactaagagaagctacgagcagatggaaaccggaggcgaacggcagaacgccacagagatcagagcctctgtgggccgtatggtcggcggcatcggcagattctacatccagatgtgcaccgaactgaagctgagcgactacgagggccgcctgatccagaacagcatcacaatcgagagaatggtgctgtccgcctttgacgagcggagaaacaaatacctggaagagcaccctagcgccggaaaagatcctaagaaaaccggcggacctatctacagaagaagagatggtaagtgggtgagagagctgattctgtacgataaggaagagattcgaagaatctggagacaggccaacaacggcgaggatgccaccgcaggcctgacacacctgatgatctggcacagcaacctgaacgatgcgacctaccagcgcacgcgggccctggtcagaaccggcatggatcctcggatgtgtagcctgatgcagggcagcacactgccaagacggagtggggccgccggcgctgcagtgaagggcgtcggaaccatggtgatggagctgatccggatgataaagcggggcatcaacgacagaaacttctggcgaggcgagaacggccgaagaacccggatcgcctacgagagaatgtgcaacatcctgaaaggaaaattccagaccgccgcccagcgggccatgatggaccaggtgcgcgagagcagaaaccccggcaatgccgagatcgaggacctgatcttcctggccagaagcgccctcattcttagaggctctgtggcccacaagagctgtctgcctgcctgtgtgtacggcctggcagtggcctcaggctacgacttcgagcgggaaggatacagtctggtgggcatcgaccctttcagactcctgcagaatagccaggtgtttagcctgatcagaccaaacgaaaaccccgcccataagagccagctggtgtggatggcctgccacagcgccgcctttgaggatctgagagtgagctcttttatcagaggcacccgggtggttccacgaggtcaactgtctacaagaggtgtgcagatcgccagcaacgagaacatggagaccatggatagcagcaccctggaactgagatccagatactgggccatcaggacacggagcggcggcaccaccaatcagcagcgcgccagcgccggccagatctctgtccagcctacgtttagcgtgcagcggaatttgcccttcgaacgcgccacaatcatggctgctttcaccggcaatacagagggcagaaccagcgatatgagaacagaaattatccgtatgatggagtccgcaaaacctgaggacgtgtccttccaaggcagaggcgtgttcgagctgagcgacgagaaggccaccaaccctatcgtgcctagcttcgatatgtctaatgagggcagctactttttcggagataacgccgaagagtacgacaacatgtctaatatggatatcgacggcattaacaccggcaccatcgacaaaacccctgaggagatcacccctggcaccagcggcacaacccggcccatcatccgccccgctacactggctccacctagcaacaagcggaccagaaatccctcgccagaaagagccacaacctccagcgaggacgacgtgggacggaagacacaaaagaagcagacccctacagagatcaagaagtctgtttacaacatggtggtgaaactgggcgagttctacaaccagatgatggtgaaggccggcctgaacgacgatatggaaagaaatctgatccagaacgcccacgccgtggagcggattctgctggccgccaccgatgataagaagaccgaattccagaaaaagaaaaacgccagagacgtgaaggaaggcaaggaagagatcgaccacaacaagacaggcggcacattctacaagatggtccgggacgacaagaccatctacttcagccctatccggataacattcctgaaagaagaagtgaagaccatgtacaaaaccacaatgggctctgacggcttcagcggcctgaatcacatcatgatcggccactctcaaatgaacgatgtgtgcttccagagaagcaaggctctgaagcgcgtgggcctggatcctagcctgatctctaccttcgccggcagcaccatccccagaagatcgggcgctaccggcgtggctatcaagggaggaggcacactggtggctgaagccatcagattcatcggaagagccatggccgacagaggactcctgagagatatcaaagccaaaaccgcctacgaaaaaatcctgctgaacctgaagaacaagtgcagcgcgcctcaacagaaggccctggtggaccaggttatcggctctagaaaccctggaatcgccgatatcgaggacctgacactgctggccagatctatggtggtggtgagaccctccgtggccagcaaggtggtgctgcctatcagcatctacgccaagatccctcagctgggatttaacgtggaagaatacagcatggttggttatgaggccatggccctgtacaacatggccacacctgtgtccatcctgagaatgggcgacgatgccaaagacaagagccagctgttcttcatgagctgcttcggcgctgcctatgaggacctgagagtgctgtccgctcttacaggaacagagttcaagcctaggagcgcactgaagtgcaagggcttccacgtgcccgccaaggaacaggtggaaggcatgggagctgctctgatgtccatcaagctgcaattttgggctcctatgacccggagcggcggaaatgaggtgggtggcgacggaggcagcggacagatttcttgcagccccgtatttgccgtggagagaccaatcgccctgtccaagcaggccgtgagaagaatgctgagcatgaacatcgagggccgggacgccgacgtgaagggcaacctgttgaagatgatgaacgacagcatggccaagaagaccagtggcaatgccttcatcggcaagaagatgttccagatctccgacaagaacaagaccaaccccgtggaaatccccatcaagcagacaatccctaacttcttcttcggcagagacaccgccgaagactatgacgacctggactacataggaaattgcccaatatgggtgaaaacacctttgaagcttgccaatggaaccaaatatagacctcctgcaaaactattaaaggaaaggggtttcttcggagctattgctggtttcctagaaggaggatgggaaggaatgattgcaggctggcacggatacacatctcacggagcacatggagtggcagtggcggcggaccttaagagtacgcaagaagctataaacaagataacaaaaaatctcaattctttgagtgagctagaagtaaagaatcttcaaagactaagtggtgccatggatgaactccacaacgaaatactcgagctggatgagaaagtggatgatctcagagctgacactataagctcgcaaatagaacttgcagtcttgctttccaacgaaggaataataaacagtgaagatgagtga

SEQ ID NO: 29 - protein, 1365 aa, Artificial Sequence
  Synthetic Construct
MPMGSLQPLATLYLLGMLVASCLGIGECPKYVRSAKLRMVTGLRNIPSIQSRGLFGAIAGFIEGGWTGMVDGWYGYHHQNEQGSGYAADQKSTQNAINGITNKVNSVIEKMNTQFTAVGKEFNKLERRMENLNKKVDDGFLDIWTYNAELLVLLENERTLDFHDSASQGTKRSYEQMETGGERQNATEIRASVGRMVGGIGRFYIQMCTELKLSDYEGRLIQNSITIERMVLSAFDERRNKYLEEHPSAGKDPKKTGGPIYRRRDGKWVRELILYDKEEIRRIWRQANNGEDATAGLTHLMIWHSNLNDATYQRTRALVRTGMDPRMCSLMQGSTLPRRSGAAGAAVKGVGTMVMELIRMIKRGINDRNFWRGENGRRTRIAYERMCNILKGKFQTAAQRAMMDQVRESRNPGNAEIEDLIFLARSALILRGSVAHKSCLPACVYGLAVASGYDFEREGYSLVGIDPFRLLQNSQVFSLIRPNENPAHKSQLVWMACHSAAFEDLRVSSFIRGTRVVPRGQLSTRGVQIASNENMETMDSSTLELRSRYWAIRTRSGGTTNQQRASAGQISVQPTFSVQRNLPFERATIMAAFTGNTEGRTSDMRTEIIRMMESAKPEDVSFQGRGVFELSDEKATNPIVPSFDMSNEGSYFFGDNAEEYDNMSNMDIDGINTGTIDKTPEEITPGTSGTTRPIIRPATLAPPSNKRTRNPSPERATTSSEDDVGRKTQKKQTPTEIKKSVYNMVVKLGEFYNQMMVKAGLNDDMERNLIQNAHAVERILLAATDDKKTEFQKKKNARDVKEGKEEIDHNKTGGTFYKMVRDDKTIYFSPIRITFLKEEVKTMYKTTMGSDGFSGLNHIMIGHSQMNDVCFQRSKALKRVGLDPSLISTFAGSTIPRRSGATGVAIKGGGTLVAEAIRFIGRAMADRGLLRDIKAKTAYEKILLNLKNKCSAPQQKALVDQVIGSRNPGIADIEDLTLLARSMVVVRPSVASKVVLPISIYAKIPQLGFNVEEYSMVGYEAMALYNMATPVSILRMGDDAKDKSQLFFMSCFGAAYEDLRVLSALTGTEFKPRSALKCKGFHVPAKEQVEGMGAALMSIKLQFWAPMTRSGGNEVGGDGGSGQISCSPVFAVERPIALSKQAVRRMLSMNIEGRDADVKGNLLKMMNDSMAKKTSGNAFIGKKMFQISDKNKTNPVEIPIKQTIPNFFFGRDTAEDYDDLDYIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDE

(19) United States
(12) Patent Application Publication
(10) Pub. No.: US 2024/0269268 A1
(43) Pub. Date: Aug. 15, 2024

(54) TEMPERATURE-CONTROLLABLE, SELF-REPLICATING RNA VACCINES FOR VIRAL DISEASES

(71) Applicant: Elixirgen Therapeutics, Inc., Baltimore, MD (US)

(72) Inventor: Minoru S.H. KO, Cockeysville, MD (US)

(73) Assignee: Elixirgen Therapeutics, Inc., Baltimore, MD (US)

(21) Appl. No.: 18/569,589

(22) PCT Filed: Jun. 17, 2022
(86) PCT No.: PCT/US2022/034104

§ 371 Date: Dec. 12, 2023

Related U.S. Application Data
(60) Provisional application No. 63/211,974, filed on Jun. 17, 2021.
(60) Provisional application No. 63/240,278, filed on Sep. 2, 2021.
(60) Provisional application No. 63/275,398, filed on Nov. 3, 2021.

(51) Int. Cl. A61K 39/295; A61K 39/00; A61K 39/145; A61K 39/215; A61P 31/14; C12N 15/86

(52) U.S. Cl. CPC A61K 39/295; A61K 39/145; A61K 39/215; A61P 31/14; C12N 15/86; A61K 2039/53; A61K 2039/54; A61K 2039/55583; A61K 2039/572; A61K 2039/575; A61K 2039/70; C07K 2319/02; C12N 2760/14122; C12N 2760/14134; C12N 2760/16122; C12N 2760/16134; C12N 2760/16222; C12N 2760/16234; C12N 2770/36122; C12N 2770/36143

(57) ABSTRACT

The present disclosure relates to mRNA, self-replicating RNA, and temperature-sensitive, self-replicating RNA encoding a coronavirus nucleocapsid protein or an influenza virus nucleocapsid protein in operable combination with a mammalian signal peptide. The present disclosure relates to mRNA, self-replicating RNA, and temperature-sensitive, self-replicating RNA encoding other viral nucleocapsid protein(s) in operable combination with a mammalian signal peptide. The RNA constructs are suitable for active immunization against a virus in a mammalian subject, such as a human subject.

### CROSS-REFERENCE TO RELATED APPLICATION(S)

This application claims the benefit of U.S. Provisional Application No. 63/275,398, filed Nov. 3, 2021, U.S. Provisional Application No. 63/240,278, filed Sep. 2, 2021, and U.S. Provisional Application No. 63/211,974, filed Jun. 17, 2021, each of which is hereby incorporated by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 699442001440SEQLIST.TXT, date recorded: Jun. 16, 2022, size: 126,113 bytes).

### FIELD

The present disclosure relates to mRNA, self-replicating RNA, and temperature-sensitive, self-replicating RNA encoding a coronavirus nucleocapsid protein or an influenza virus nucleocapsid protein in operable combination with a mammalian signal peptide. The present disclosure relates to mRNA, self-replicating RNA, and temperature-sensitive, self-replicating RNA encoding other viral nucleocapsid protein(s) in operable combination with a mammalian signal peptide. The RNA constructs are suitable for active immunization against a virus in a mammalian subject, such as a human subject.

### BACKGROUND

The betacoronavirus genus encompasses Severe Acute Respiratory Syndrome (SARS)-COV-2, which caused the COVID-19 pandemic, SARS-COV-1, which caused the 2002-2004 SARS outbreak, and Middle East Respiratory Syndrome (MERS)-CoV. The COVID-19 pandemic has made design and production of vaccines an urgent necessity for immunization of a large global population.

The SARS-COV-2 vaccines currently approved by the U.S. Food & Drug Administration are designed to elicit neutralizing antibodies (nAb) against the Spike (S) protein or the receptor binding domain (RBD) of the S protein in advance of infection. However, this approach poses a great challenge in that the S protein is not well conserved even between SARS-CoV-1 and SARS-COV-2 strains. In particular, small amino acid changes that occur among variants often result in conformational changes to the S protein that may significantly reduce the effectiveness of nAb elicited by the specific S protein of the COVID-19 vaccine.

Continued vaccine development targeting only the betacoronavirus S protein is therefore contemplated to follow the path of seasonal influenza vaccines. This means that the continual emergence of variants will likely require development and production of new vaccines on a periodic basis. Although annual production of betacoronavirus vaccines may be technically feasible, global vaccination efforts involving annual administration of new vaccines are economically and logistically impractical. The problems posted by annual administration of new vaccines present especially undue burdens for low- and middle-income countries.

Accordingly, there is a need in the art for betacoronavirus vaccines that safely induce a long-lived, immune response that is broadly reactive against SARS-COV-2 variants. Preferably the long-lived, immune response is broadly reactive with other betacoronaviruses, which cause disease in humans. There is also a need in the art for influenza virus vaccines that are safe and effective in inducing a broadly reactive immune response against influenza A and/or influenza B viruses.

### BRIEF SUMMARY

The present disclosure relates to the use of nucleoproteins (also referred to herein as nucleocapsid proteins) from betacoronaviruses as a vaccine antigen to induce cellular immune responses that are broadly reactive with betacoronavirus variants. In some embodiments, a temperature-controllable, self-replicating RNA (referred to herein as srRNAts and c-srRNA) vaccine platform is utilized. The c-srRNA vaccine platform is advantageous for induction of a potent cellular immune response after intradermal administration. In some embodiments, a nucleoprotein from SARS-COV-2 is expressed in host cells to address infection by both SARS-CoV-2 and SARS-COV-1, as well as variants thereof. In some embodiments, a nucleoprotein from a coronavirus is fused with a signal peptide of the human CD5 antigen and expressed in host cells to enhance the cellular immune response elicited against the coronavirus. In some embodiments, a nucleoprotein from a first coronavirus is fused to a nucleoprotein from a second coronavirus, which is different from the first coronavirus. In some embodiments, the fusion protein comprises a tandem array of two or three coronavirus nucleoproteins. In a subset of these embodiments, the fusion protein comprises a SARS-COV-2 nucleoprotein and a MERS-CoV nucleoprotein. In some embodiments, the fusion protein further comprises a coronavirus spike protein or fragment thereof. In this way, a more broadly reactive coronavirus-specific immune response is stimulated.

The present disclosure also relates to the use of nucleoproteins (also referred to herein as nucleocapsid proteins) from influenza viruses as a vaccine antigen to induce cellular immune responses that are broadly reactive with influenza A and/or influenza B viruses, which rapidly change over time as a consequence of antigen drift and antigen shift. In some embodiments, a temperature-controllable, self-replicating RNA vaccine platform is utilized. The c-srRNA vaccine platform is advantageous for induction of a potent cellular immune response after intradermal administration. In some embodiments, a nucleoprotein from one subtype of influenza A (FluA) virus is expressed in host cells to address infection by the same and different subtypes of FluA. In some embodiments, a nucleoprotein from one lineage of influenza B (FluB) virus is expressed in host cells to address infection by the same and different lineages of FluB. In some embodiments, a nucleoprotein from an influenza virus is fused with a signal peptide of the human CD5 antigen and expressed in host cells to enhance the cellular immune response elicited against the influenza virus. In some embodiments, a nucleoprotein from a FluA virus is fused to a nucleoprotein from a FluB virus. In some embodiments, the fusion protein comprises a tandem array of two or three nucleoproteins from one or more strains of FluA and/or one or more lineages of FluB. In some embodiments, the fusion protein further comprises an influenza hemagglutinin or fragment thereof. In this way, a more broadly reactive influenza-specific immune response is stimulated.

The present disclosure also relates to the use of nucleoproteins (also referred to herein as nucleocapsid proteins) from ebolaviruses as a vaccine antigen to induce cellular immune responses that are broadly reactive with two, three or four species of ebolavirus that infect humans. In some embodiments, a temperature-controllable, self-replicating RNA vaccine platform is utilized. The c-srRNA vaccine platform is advantageous for induction of a potent cellular immune response after intradermal administration. In some embodiments, a nucleoprotein from an ebolavirus is fused with a signal peptide of the human CD5 antigen and expressed in host cells to enhance the cellular immune response elicited against the ebolavirus. In some embodiments, a nucleoprotein from a first ebolavirus species is fused to a nucleoprotein from a second ebolavirus species, which is optionally fused to a nucleoprotein of a third ebolavirus species, which is optionally fused to a nucleoprotein of a fourth ebolavirus species. In some embodiments, the fusion protein comprises a tandem array of two, three or four nucleoproteins or fragments thereof from two or more species of ebolavirus. In some embodiments, the fusion protein further comprises an ebolavirus envelope glycoprotein or fragment thereof. In this way, a more broadly reactive ebolavirus-specific immune response is stimulated.

Among other embodiments, the present disclosure provides compositions comprising an excipient and a temperature-controllable, self-replicating RNA. In some embodiments, the composition comprises a chitosan. In some embodiments, the chitosan is a low molecular weight (about 3-5 kDa) chitosan oligosaccharide, such as chitosan oligosaccharide lactate. In some embodiments, the composition does not comprise liposomes or lipid nanoparticles.

### BRIEF DESCRIPTION OF THE DRAWINGS

FIG. 1 shows a schematic of the mechanism for induction of cellular (CD4+ and CD8+ T cell) immune responses after intradermal injection of a temperature-controllable, self-replicating RNA (referred to herein as srRNAts and c-srRNA) vaccine.

FIG. 2 shows a schematic diagram of SARS-COV-2 nucleocapsid (N) proteins expressed from mRNA, self-replicating RNA, or temperature-sensitive, self-replicating RNA (srRNAts) delivered to mammalian host cells. In exemplary embodiments, the coding region of the N protein is the gene of interest (GOI) inserted within the srRNAts. The amino acid sequence of the G5004 antigen is set forth as SEQ ID NO:5. The G5004 antigen is a SARS-CoV-2 N protein devoid of a signal peptide. The amino acid sequence of the G5005 antigen is set forth as SEQ ID NO:6. The G5005 antigen is a fusion protein comprising the signal peptide sequence from the human CD5 antigen (CD5-SP) set forth as SEQ ID NO:8, and a SARS-COV-2 N protein, in which the CD5-SP replaces the start methionine at position 1 of the N protein. The amino acid sequence of the G5006 antigen is set forth as SEQ ID NO:7. The G5006 antigen is a fusion protein comprising the signal peptide sequence from CD5-SP, a SARS-COV-2 N protein, and a MERS-COV N protein. The nucleotide sequence encoding the G5004 antigen is set forth as SEQ ID NO:1. The nucleotide sequence encoding the G5005 antigen is set forth as SEQ ID NO:2. The nucleotide sequence encoding the G5006 antigen is set forth as SEQ ID NO:3, and as a codon-optimized version in SEQ ID NO:4.

FIG. 3 shows a schematic diagram of an exemplary method for stimulating an immune response against a coronavirus in a human subject. A temperature-sensitive agent (ts-agent) such as a srRNAts is functional at a permissive temperature, but is non-functional at a non-permissive temperature. The temperature at or just below the surface of a human subject's body (surface body temperature) is a permissive temperature, while the human subject's core body temperature is a higher, non-permissive temperature. Thus, a ts-agent administered intradermally to the human subject is functional while located at the permissive temperature just below the surface of the human subject's body.

FIG. 4A and FIG. 4B show the frequency of cytokine-secreting cells in samples of splenocytes obtained from CD-1 outbred mice that had been immunized by a single intradermal injection of 100 μL solution containing either 5 μg or 25 μg of a temperature-controllable self-replicating RNA (srRNA1ts2 [PCT/US20/67506]) encoding the G5004 antigen or a placebo (PBO: buffer only). FIG. 4A shows the frequency of interferon-gamma (INF-γ) spot-forming cells (SFC) and FIG. 4B shows the frequency of interleukin-4 (IL-4) SFC in 1×10\hat{ }6 splenocytes after restimulation by culturing the splenocytes in the presence or absence of a pool of SARS-COV-2 nucleoprotein peptides. The frequency obtained in the presence of peptides is plotted in the graph after subtracting the frequency obtained in the absence of peptides (background). The average and standard deviation (error bars) of five mice (n=5) are shown for each group. Splenocytes were isolated 14 days after intradermal injection.

FIG. 5A and FIG. 5B show the frequency of cytokine-secreting cells in samples of splenocytes obtained from CD-1 outbred mice that had been immunized by a single intradermal injection of 100 μL solution containing either 5 μg or 25 μg of a temperature-controllable self-replicating RNA (srRNA1ts2 [PCT/US20/67506]) encoding the G5005 antigen or a placebo (PBO: buffer only). FIG. 5A shows the frequency of interferon-gamma (INF-γ) spot-forming cells (SFC) and FIG. 5B shows the frequency of interleukin-4 (IL-4) SFC in 1×10\hat{ }6 splenocytes after restimulation by culturing the splenocytes in the presence or absence of a pool of SARS-COV-2 nucleoprotein peptides. The frequency obtained in the presence of peptides is plotted in the graph after subtracting the frequency obtained in the absence of peptides (background). The average and standard deviation (error bars) of five mice (n=5) are shown for each group. Splenocytes were isolated 14 days after intradermal injection.

FIG. 6A and FIG. 6B show the frequency of cytokine-secreting cells in samples of splenocytes obtained from BALB/c mice that had been immunized by a single intradermal injection of 100 μL solution containing either 5 μg or 25 μg of a temperature-controllable self-replicating RNA (srRNA Its2 [PCT/US20/67506]) encoding the G5005 antigen or a placebo (PBO: buffer only). FIG. 6A shows the frequency of interferon-gamma (INF-y) spot-forming cells (SFC) and FIG. 6B shows the frequency of interleukin-4 (IL-4) SFC in 1×10\hat{ }6 splenocytes after restimulation by culturing the splenocytes in the presence or absence of a pool of SARS-CoV-2 nucleoprotein peptides. The frequency obtained in the presence of peptides is plotted in the graph after subtracting the frequency obtained in the absence of peptides (background). The average and standard deviation (error bars) of five mice (n=5) are shown for each group. Splenocytes were isolated 30 days after the vaccination.

FIG. 7 show the levels of SARS-COV-2 antigen-reactive immunoglobulin G (IgG) in serum of BALB/c mice that had been immunized by a single intradermal injection of a 100 μL solution containing either 5 μg or 25 μg of a temperature-controllable self-replicating RNA (srRNAlts2 [PCT/US20/67506]) encoding the G5005 antigen or a placebo (PBO: buffer only). The IgG levels are represented by OD450 in the ELISA. The IgG levels before (Day -1) and after (Day 30) vaccination (Day 0) are shown. The average and standard deviation (error bars) of five mice (n=5) are shown for each group.

FIG. 8 shows the frequency of interferon-gamma (INF-y)-secreting cells in samples of splenocytes obtained from BALB/c mice that had been immunized by a single intradermal injection of 100 μL solution containing either 5 μg or 25 μg of a temperature-controllable self-replicating RNA (srRNA Its2 [PCT/US20/67506]) encoding the G5006 antigen or a placebo (PBO: buffer only). Specifically, FIG. 8 shows the frequency of INF-γ spot-forming cells (SFC) in 1×10\hat{ }6 splenocytes after restimulation by culturing the splenocytes in the presence or absence of a pool of SARS-COV-2 nucleoprotein peptides. The frequency obtained in the presence of peptides is plotted in the graph after subtracting the frequency obtained in the absence of peptides (background). The average and standard deviation (error bars) of five mice (n=5) are shown for each group. Splenocytes were isolated 14 days after the vaccination.

FIG. 9 shows a schematic diagram of an exemplary pan-influenza vaccine. In brief, a fusion protein comprising a nucleoprotein from an Influenza Type A virus (FluA) and a nucleoprotein from an Influenza Type B virus (FluB) is expressed from mRNA, self-replicating RNA, or temperature-sensitive, self-replicating RNA (srRNAts) delivered to mammalian host cells. In exemplary embodiments, the coding region of the fusion protein is the gene of interest (GOI) inserted within the srRNAts. Specifically, G5010 is a fusion protein comprising the signal peptide sequence from the human CD5 antigen (CD5-SP) set forth as SEQ ID NO:8, the FluA nucleoprotein (Influenza Type A, H5N8 subtype [A/breeder duck/Korea/Gochang1/2014], GenBank No. KJ413835.1, ProteinID No. AHL21420.1), and the FluB nucleoprotein (Influenza Type B [B/Florida/4/2006], GenBank No. CY033879.1, ProteinID No. ACF54251.1). In G5010, the CD5-SP replaces the start methionine of the FluA nucleoprotein, and the FluA nucleoprotein is fused to the methionine of the start codon of the FluB nucleoprotein.

FIG. 10 shows an alignment of the nucleoprotein of Influenza A (H5N8 strain; ProteinID AHL21420.1) used as a vaccine antigen in G5010 (SEQ ID NO:13) and the nucleoprotein of Influenza A (NP/AnnArbor H2N2; ProteinID P21433) used as a source (SEQ ID NO:17) of a peptide pool for ELISpot assay.

FIG. 11A and FIG. 11B show the frequency of cytokine-secreting cells in samples of splenocytes obtained from BALB/c mice that had been immunized by a single intradermal injection of 100 μL solution containing either 5 μg or 25 μg of a temperature-controllable self-replicating RNA (srRNA1ts2 [PCT/US20/67506]) encoding the G5010 antigen or a placebo (PBO: buffer only). FIG. 11A shows the frequency of interferon-gamma (INF-γ) spot-forming cells (SFC) and FIG. 11B shows the frequency of interleukin-4 (IL-4) SFC in 1×10\hat{ }6 splenocytes after restimulation by culturing the splenocytes in the presence or absence of a pool of SARS-COV-2 nucleoprotein peptides. The frequency obtained in the presence of peptides is plotted in the graph after subtracting the frequency obtained in the absence of peptides (background). The average and standard deviation (error bars) of five mice (n=5) are shown for each group. Splenocytes were isolated 14 days after the vaccination.

FIG. 12 shows a schematic diagram of an exemplary pan-ebolavirus vaccine. In brief, a fusion protein comprising nucleoproteins of four different ebolavirus strains is expressed from mRNA, self-replicating RNA, or temperature-sensitive, self-replicating RNA (srRNAts) delivered to mammalian host cells. In exemplary embodiments, the coding region of the fusion protein is the gene of interest (GOI) inserted within the srRNAts. Specifically, the exemplary PanEbola antigen is a fusion protein comprising the signal peptide sequence from the human CD5 antigen (CD5-SP) set forth as SEQ ID NO:8, a part of a nucleoprotein of Zaire ebolavirus (residues 2-739; total 738 aa; GenBank ID: AF272001) set forth as SEQ ID NO:18, a part of a nucleoprotein of Sudan ebolavirus (residues 403-738; total 336 aa; GenBank ID: AF173836) set forth as SEQ ID NO: 19, a part of a nucleoprotein of Bundibugyo ebolavirus (residues 403-739; total 337 aa; GenBank ID: FJ217161) set forth as SEQ ID NO:20, and a part of a nucleoprotein of Taï Forest ebolavirus (residues 483-651; total 169 aa; GenBank ID: FJ217162) set forth as SEQ ID NO:21. The amino acid sequence of the PanEbola antigen is set forth as SEQ ID NO:22, while the nucleic acid sequence encoding the PanEbola antigen is set forth as SEQ ID NO:23.

FIG. 13 shows amino acid sequence similarities among four species of Ebolavirus as percent identities. Amino acid sequence of Zaire ebolavirus NP (GenBank ID: AF272001), Sudan ebolavirus NP (GenBank ID: AF173836), Bundibugyo ebolavirus NP (GenBank ID: FJ217161), Taï Forest ebolavirus NP (GenBank ID: FJ217162) were compared to each other by using NCBI BlastP algorithm. Based on the sequence alignment, proteins were divided into well-conserved regions (A) and less well-conserved regions (B). The amino acid sequence identity between Zaire ebolavirus NP and Sudan ebolavirus NP was 88% for Region A, whereas it was 42% for Region B. The amino acid sequence identity between Zaire ebolavirus NP and Bundibugyo ebolavirus NP was 92% for Region A, whereas it was 53% for Region B. The amino acid sequence identity between Zaire ebolavirus NP and Taï Forest ebolavirus NP was 92% for Region A, whereas it was 54% for Region B. For Region B, Bundibugyo (B) and Taï Forest (B) sequences shared a relatively high level of sequence similarity. Based on the sequence alignment of Region B, proteins were divided into well-conserved regions (80% and 86% similarity; no label) and a less well-conserved region (40% identity; referred to herein as Region C).

FIG. 14A and FIG. 14B show the frequency of cytokine-secreting cells in samples of splenocytes obtained from BALB/c mice that had been immunized by a single intradermal injection of 100 μL solution containing either 25 μg of a temperature-controllable self-replicating RNA (srRNAlts2 as described in WO 2021/138447 A1, also called c-srRNA) encoding the PanEbola antigen (srRNAlts2-PanEbola, also called G5011) or a placebo (PBO: buffer only). FIG. 14A shows the frequency of interferon-gamma (INF-γ) spot-forming cells (SFC) and FIG. 14B shows the frequency of interleukin-4 (IL-4) SFC in 1×10\hat{ }6 splenocytes after restimulation by culturing the splenocytes in the presence or absence of a pool of 182 peptides derived from a peptide scan (15mers with 11 amino acid overlaps) through Nucleoprotein (Swiss-Prot ID: B8XCN6) of Taï Forest Ebolavirus [JPT peptide; PepMix Tai Forest Ebolavirus (NP); JPT Product Code: PM-TEBOV-NP]. The frequency obtained in the presence of peptides is plotted in the graph after subtracting the frequency obtained in the absence of peptides (background). The average and standard deviation (error bars) of five mice (n=5) are shown for each group. Splenocytes were isolated 14 days after the vaccination.

FIG. 15 depicts a schematic diagram showing exemplary srRNAlts2 constructs encoding the receptor binding domain (RBD) of the spike protein of severe acute respiratory syndrome coronavirus-2 (SARS-COV-2). G5003 is the same antigen as “srRNAlts2-2019-nCOV-RBD1” presented in FIG. 21 of WO 2021/138447 A1; and G5003 encodes a fusion protein including the signal peptide of CD5 (residues 1-24) and the RBD of the spike protein of SARS-CoV-2 (an original Wuhan strain). G50030 encodes a fusion protein (SEQ ID NO:25) including the signal peptide of CD5 (residues 1-24) and the RBD of the spike protein of SARS-COV-2 (an omicron strain B.1.1.529: Science Brief: Omicron (B.1.1.529) Variant | CDC). The nucleotide sequence of the G50030 open reading frame is set forth as SEQ ID NO:24.

FIG. 16A and FIG. 16B show the frequency of cytokine-secreting cells in samples of splenocytes obtained from C57BL/6 mice that had been immunized by a single intradermal injection of 100 μL solution containing either placebo (PBO: buffer only) or 25 μg of a temperature-controllable self-replicating RNA (srRNAlts2 as described in WO 2021/138447 A1) encoding the G50030 antigen. FIG. 16A shows the frequency of interferon-gamma (INF-γ) spot-forming cells (SFC) and FIG. 16B shows the frequency of interleukin-4 (IL-4) SFC in 1×10\hat{ }6 splenocytes from immunized mice restimulated by culturing in the presence or absence of a pool of 53 peptides derived from a peptide scan (15mers with 11 amino acid overlaps) through RBD of SARS-COV-2 omicron variant (B.1.1.529) [JPT peptide Product Code: PM-SARS2-RBDMUT08-1]. The assays were performed by the ELISpot assay. The frequency obtained in the presence of peptides is plotted in the graph after subtracting the frequency obtained in the absence of peptides (background). The average and standard deviation (error bars) of five mice (n=5) are shown for each group. Splenocytes were isolated 14 days after the vaccination.

FIG. 17A-17C show the induction of both cellular immunity and humoral

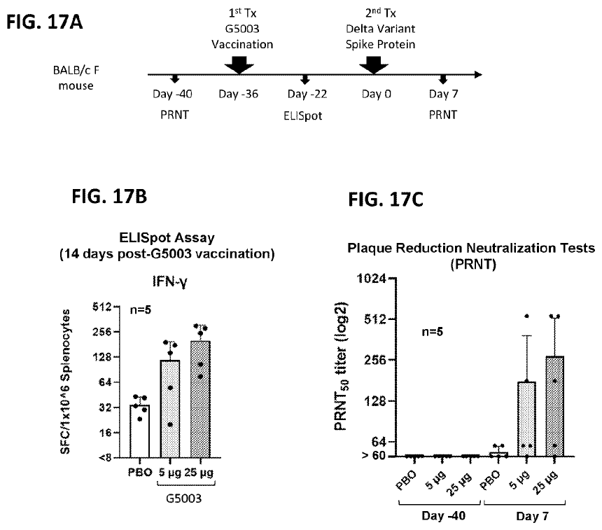

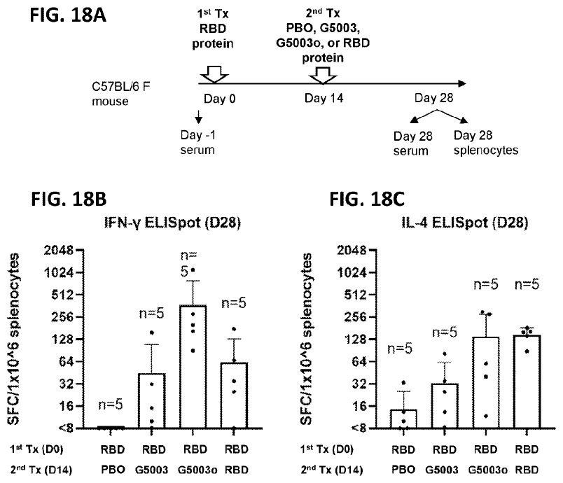

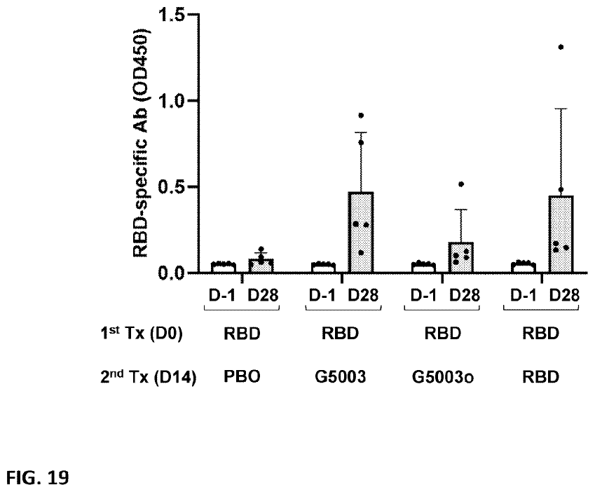

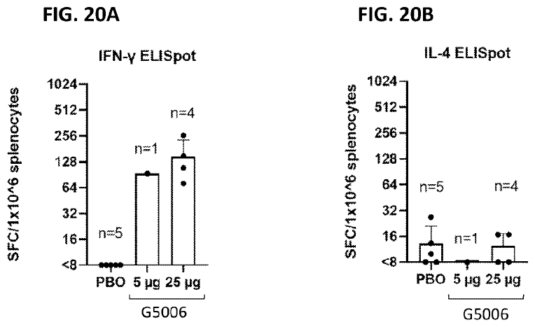

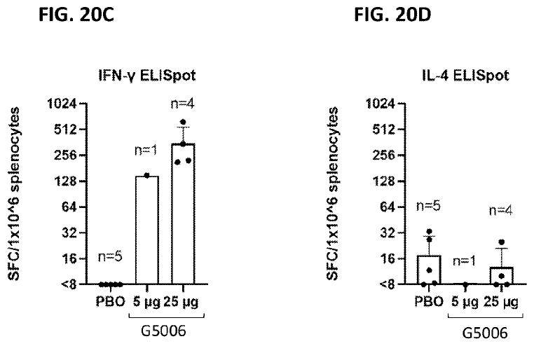

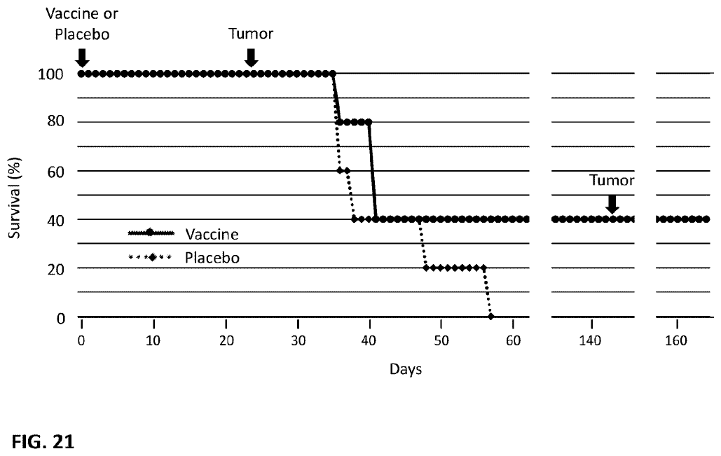

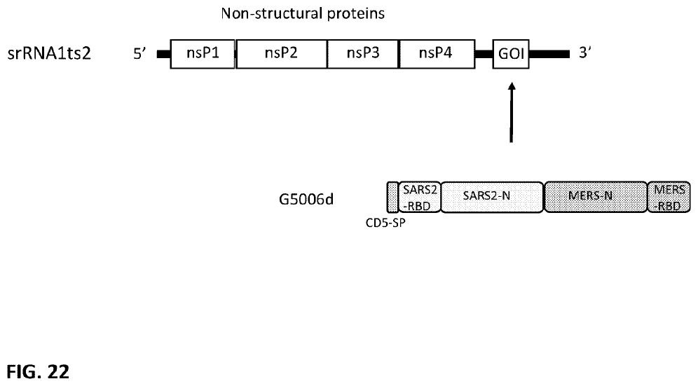

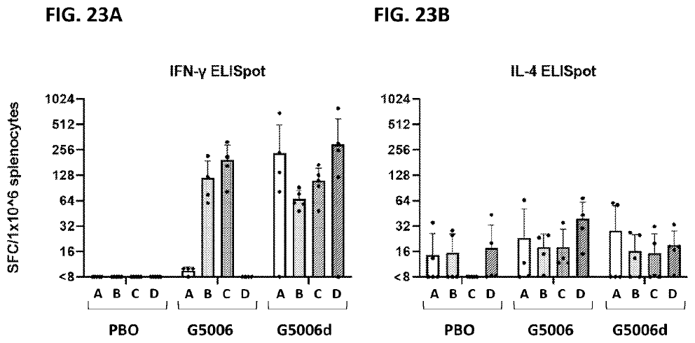

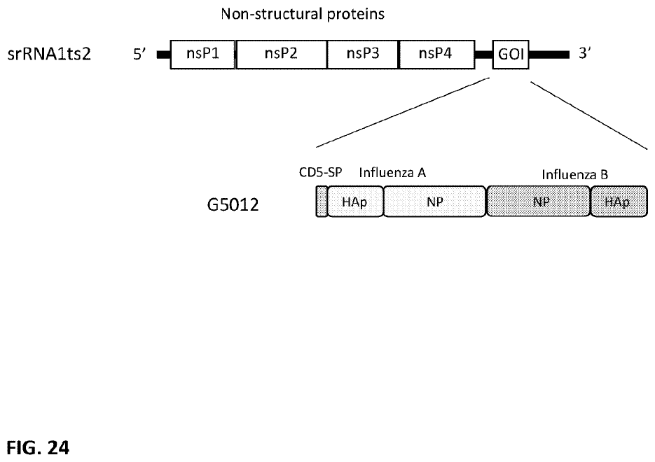

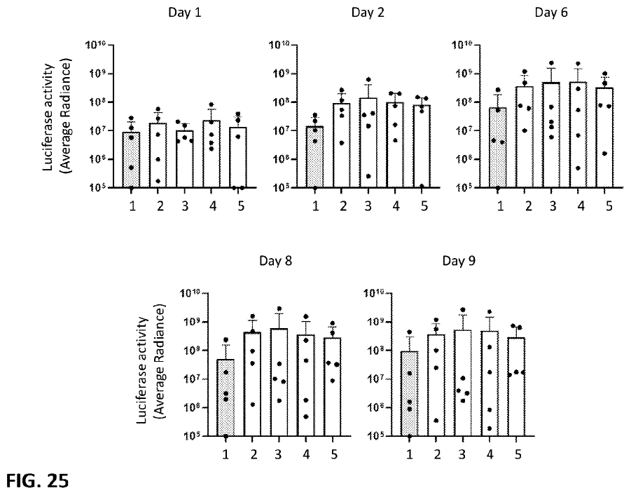

immunity in mice as a consequence of administering a composition comprising a c-srRNA encoding an antigen, followed by administering a composition comprising a protein antigen. FIG. 17A depicts a schematic diagram of experimental procedures. On day -40, blood was withdrawn from female BALB/c mice for the plaque reduction neutralization test (PRNT). On day -36, these mice were treated with c-srRNA encoding G5003 antigen. The c-srRNA was injected intradermally into mouse skin as a naked RNA, without any nanoparticle nor transfection reagent. On day -22 (14 days after c-srRNA-G5003 vaccination), a half of mice were sacrificed to obtain splenocytes for ELISpot assays. On day 0, the remaining mice were intradermally injected with a Spike protein of SARS-COV-2 Delta variant (B.1.617.2) mixed with adjuvant (Adda Vax™ adjuvant marketed by Invivogen). On day 7 (7 days after the Spike protein injection), blood was withdrawn for the PRNT assays. FIG. 17B shows the induction of cellular immunity against the RBD protein by a single intradermal vaccination with the c-srRNA-G5003 vaccine. The figure shows the frequency of interferon-gamma (INF-γ) spot-forming cells (SFC) in 1×10\hat{ }6 splenocytes from immunized mice restimulated by culturing in the presence or absence of a pool of 53 peptides (15 mers with 11 amino acid overlaps) that covers SARS-COV-2 RBD (an original Wuhan strain). The assays were performed by the ELISpot assay. The frequency obtained in the presence of peptides is plotted in the graph after subtracting the frequency obtained in the absence of peptides (background). The average and standard deviation (error bars) of five mice (n=5) are shown for each group. Splenocytes were isolated on day -22 (14 days after the vaccination). FIG. 17C shows the titer of serum antibodies that can neutralize (50%) the SARS-COV-2 virus (Delta variant B.1.617.2), measured by a plaque reduction neutralization assay (PRNT). Exposure to a spike protein of SARS-COV-2 virus (Delta variant B.1.617.2) induced neutralization antibodies specifically against the Delta variant of SARS-COV-2 virus only in mice vaccinated with a vaccine c-srRNA-G5003, encoding the RBD of the SARS-COV-2 (an original Wuhan strain).

FIG. 18A -- 18C show the induction of cellular immunity in mice as a consequence of administering a composition comprising a protein antigen, followed by administering a composition comprising c-srRNA encoding an antigen. FIG. 18A depicts a schematic diagram of experimental procedures. On day 0 (1st treatment), female C57BL/6 mice were treated with intradermal injection with 10 μg RBD protein (Sino Biological SARS-COV-2 [2019-nCOV]) +Adjuvant (Adda Vax™ adjuvant marketed by Invivogen). On day 14 (2nd treatment), the mice were treated with intradermal injection of a placebo (PBO: buffer only), 25 ug c-srRNA encoding G5003 antigen, 25 μg c-srRNA encoding G50030 antigen, or 10 μg RBD protein (Sino Biological SARS-COV-2 [2019-nCOV]) +Adjuvant (Adda Vax™ adjuvant). On day 28, mice were sacrificed, and splenocytes and serum were collected. FIG. 18B shows the frequency of interferon-gamma (INF-γ) and FIG. 18C shows the frequency of interleukin 4 (IL-4) spot-forming cells (SFC) in 1×10\hat{ }6 splenocytes restimulated by culturing in the presence or absence of a pool of 53 peptides (15 mers with 11 amino acid overlaps) that covers SARS-COV-2 RBD (an original Wuhan strain). The assays were performed by the ELISpot assay. The frequency obtained in the presence of peptides is plotted in the graph after subtracting the frequency obtained in the absence of peptides (background).

FIG. 19 shows the level of serum antibodies against the RBD of the SARS-COV-2 virus (an original Wuhan strain) as determined by an ELISA assay (represented by the OD450 measurement). The average and standard deviation (error bars) of five mice (n=5) are shown for each group. The data of Day -1 (before the 1st treatment) and the data of Day 28 (after the 2nd treatment) are shown for each group.

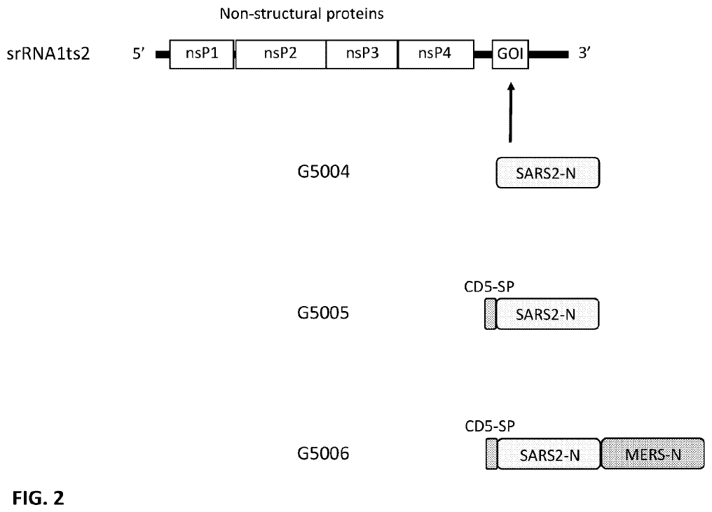

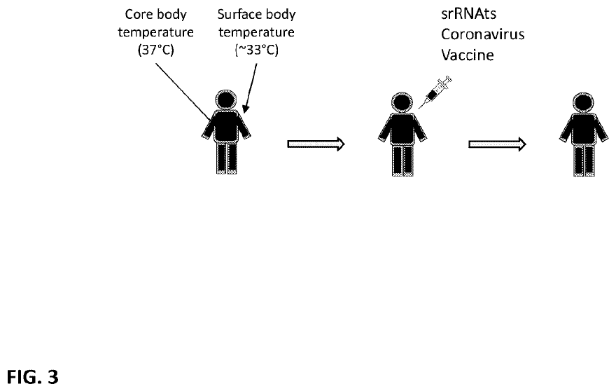

FIGS. 20A-D show the frequency of interferon-gamma (INF-γ)- or interleukin 4 (IL-4)-secreting cells in samples of splenocytes obtained from BALB/c mice that had been immunized by a single intradermal injection of 100 μL solution containing either 5 μg (n=1) or 25 μg (n=4) of a temperature-controllable self-replicating RNA (srRNA1ts2 as described in WO 2021/138447 A1) encoding the G5006 antigen (FIG. 2) or a placebo (PBO: buffer only:

n=5). The frequency obtained in the presence of peptides is plotted in the graph after subtracting the frequency obtained in the absence of peptides (background). The average and standard deviation (error bars) of one mouse (n=1) or four mice (n=4) are shown for each group. Splenocytes were isolated 14 days after the vaccination. FIG. 20A and FIG. 20B show the results after restimulation by culturing the splenocytes in the presence or absence of a pool of SARS-COV-2 nucleoprotein peptides. FIG. 20C and FIG. 20D show the results after restimulation by culturing the splenocytes in the presence or absence of a pool of MERS-COV-2 nucleoprotein peptides.

FIG. 21 shows survival (%) of the female BALB/c mice vaccinated with c-srRNA-G5006, followed by the injection of tumor cells expressing G5006 antigens.

FIG. 22 depicts a schematic diagram showing exemplary srRNAlts2 constructs encoding a fusion protein of the signal peptide of CD5 (residues 1-24), the receptor binding domain (RBD) of the spike protein of severe acute respiratory syndrome coronavirus-2 (SARS-CoV-2), the nucleoprotein of SARS-COV-2, the nucleoprotein of MERS-COV, and the RBD of MERS-COV (named here G5006d). The amino acid sequence of the pan-coronavirus antigen (G5006d) is set forth as SEQ ID NO:27, and the nucleotide sequence of its open reading frame is set forth as SEQ ID NO:26.

FIGS. 23A-B show the frequency of cytokine-secreting cells in samples of splenocytes obtained from female C57BL/6 mice that had been immunized by a single intradermal injection of 100 μL solution containing either placebo (PBO: buffer only), 25 μg of a temperature-controllable self-replicating RNA (srRNAIts2 as described in WO 2021/138447 A1) encoding the G5006 antigen, or 25 μg of a temperature-controllable self-replicating RNA (srRNAlts2 as described in WO 2021/138447 A1) encoding the G5006d antigen. FIG. 23A shows the frequency of interferon-gamma (INF-γ) spot-forming cells (SFC) and FIG. 23B shows the frequency of interleukin-4 (IL-4) SFC in 1×10\hat{ }6 splenocytes from immunized mice restimulated by culturing in the presence or absence of pools of peptides derived from a peptide scan (15mers with 11 amino acid overlaps) through (A) RBD of Spike protein of SARS-COV-2 [JPT Peptide Product Code: PM-WCPV-S-RBD-2]; (B) Nucleoprotein of SARS-COV-2 [JPT peptide Product Code: PM-WCPV-NCAP]; (C) Nucleoprotein of MERS-COV [JPT peptide, custom made]; and (D) Spike protein of MERS-COV [JPT peptide Product Code: PM-MERS-CoV-S-1). The assays were performed by the ELISpot assay. The frequency obtained in the presence of peptides is plotted in the graph after subtracting the frequency obtained in the absence of peptides (background). The average and standard deviation (error bars) of five mice (n=5) for PBO, four mice (n=4) for G5006, and five mice (n=5) for G5006d, are shown for each group. Splenocytes were isolated 14 days after the vaccination.

FIG. 24 depicts a schematic diagram showing exemplary srRNAlts2 constructs encoding a fusion protein (G5012) of the signal peptide of CD5 (residues 1-24), a part of the hemagglutinin (HA) of the Influenza A (A/New Caledonia/20/1999(H1N1)) (residues 25-165), nucleoprotein of Influenza A (A/breeder duck/Korea/Gochang1/2014(H5N8)) (residues 166-662), nucleoprotein of Influenza B (B/Florida/4/2006) (residues 663-1222), and a part of the hemagglutinin (HA) of the Influenza B (B/Florida/4/2006) (residues 1223-1365). The amino acid sequence of the pan-influenza virus antigen (G5012) is set forth as SEQ ID NO:29, and the nucleotide sequence of its open reading frame is set forth as SEQ ID NO:28.

FIG. 25 shows the effects of Chitosan Oligomers on gene (luciferase) expression from srRNAlts2 (exemplary c-srRNA) in mice. c-srRNA encoding luciferase was intradermally injected into mice under the following conditions: 1, a control - c-srRNA only; 2, c-srRNA mixed with chitosan oligosaccharide (0.001 μg/mL); 3, c-srRNA mixed with chitosan oligosaccharide (0.01 μg/mL); 4, c-srRNA mixed with chitosan oligosaccharide (0.5 μg/mL); and 5, c-srRNA mixed with chitosan oligosaccharide lactate (0.1 μg/mL).

### DETAILED DESCRIPTION

Broader, longer-lasting protection against SARS-COV-1, SARS-COV-2, MERS-CoV, and their variants, is best achieved through vaccines that induce cellular immunity (i.e., T-cell-inducing vaccines involving CD8+killer T cells and CD4+helper T cells). This is a departure from the current, neutralizing antibody-focused COVID-19 vaccine paradigm, as discussed in the Background section. The critical importance of cellular immunity in fighting against coronaviruses has been demonstrated experimentally and extensively discussed [Sette and Crotty 2021]. Cellular immunity alone can provide protection via CD8+killer T cells [Matchett et al., 2021]. Also, cellular immunity depends on linear T cell epitopes, whereas humoral immunity depends on conformational (as well as linear) B cell epitopes. Therefore, cellular immunity is much more robust against variants than humoral immunity. Furthermore, memory T cells last longer than memory B cells, and thus, potentially provide lifelong immunity. This requires both suitable antigens and a cellular immunity-based vaccine platform.

### Cellular Immunity-Based mRNA Vaccine Platform

The vaccine platform is described in Elixirgen's earlier patent application [PCT/US20/67506, now published as WO 2021/138447 A1]. This vaccine platform is optimized to induce cellular immunity, which becomes possible by combining existing knowledge of vaccine biology with temperature-controllable self-replicating mRNA (srRNAts) based on an Alphavirus, such as the Venezuelan equine encephalitis virus (VEEV). The terms c-srRNA and srRNAts are used interchangeably throughout the present disclosure, with srRNAlts2 (described in WO 2021/138447 A1) being an exemplary embodiment. srRNAts is based on srRNA, also known as self-amplifying mRNA (saRNA or SAM), by incorporation of small amino acid changes in the Alphavirus replicase that provide temperature-sensitivity. Elixirgen Therapeutic Inc.'s srRNAts is functional at 30-35° C., but not functional at or above 37° C.±0.5° C. It carries all the benefits of mRNA platforms: no genome integration, rapid development and deployment, and a simple good manufacturing process (GMP), as well as additional advantages of srRNA platforms compared to mRNA platforms, particularly longer expression [Johanning et al., 1995] and higher immunogenicity at a lower dosage [Brito et al., 2014]. However, this simple temperature-controllable feature makes it possible to pull together many desirable features of T-cell inducing vaccine as described herein.

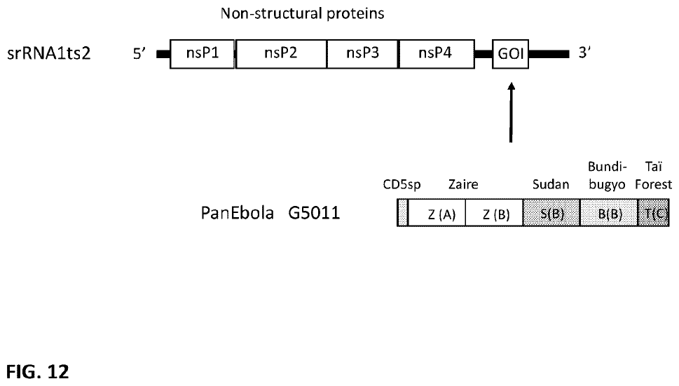

In brief, srRNAlts2 is a temperature-sensitive, self-replicating VEEV-based RNA replicon developed for transient expression of a heterologous protein. Temperature-sensitivity is conferred by an insertion of five amino acids residues within the non-structural Protein 2 (nsP2) of VEEV. The nsP2 protein is a helicase/proteinase, which along with nsP1, nsP3 and nsP4 constitutes a VEEV replicase. srRNAlts2 does not contain VEEV structural proteins (capsid, El, E2 and E3). The disclosure of WO 2021/138447 A1 of Elixirgen Therapeutics, Inc. is hereby incorporated by reference. In particular, Example 3, FIG. 12, and SEQ ID NOs. 29-49 of WO 2021/138447 A1 are hereby incorporated by reference.

Overall, the srRNAts platform's compelling potential for immunogenicity (dose-sparing) and safety benefits (temperature-control and naked delivery), provisioning of long-lasting baseline cellular immunity, and ability to provide rapid humoral responses across variants makes it a strong candidate for large-scale deployment to meet the global need for an inexpensive, safe, variant-addressing vaccine that provides long-term immunity.

### General Techniques and Definitions

The practice of the present disclosure will employ, unless otherwise indicated, conventional techniques of molecular biology (including recombinant techniques), microbiology, cell biology, biochemistry and immunology, which are within the skill of the art.

As used herein and in the appended claims, the singular forms “a”, “an”, and “the” include plural references unless indicated otherwise. For example, “an” excipient includes one or more excipients.

The phrase “comprising” as used herein is open-ended, indicating that such embodiments may include additional elements. In contrast, the phrase “consisting of” is closed, indicating that such embodiments do not include additional elements (except for trace impurities). The phrase “consisting essentially of” is partially closed, indicating that such embodiments may further comprise elements that do not materially change the basic characteristics of such embodiments.

The term “about” as used herein in reference to a value, encompasses from 90% to 110% of that value (e.g., molecular weight of about 5,000 daltons when used in reference to a chitosan oligosaccharide refers to 4,500 daltons to 5,500 daltons).

The term “antigen” refers to a substance that is recognized and bound specifically by an antibody or by a T cell antigen receptor. Antigens can include peptides, polypeptides, proteins, glycoproteins, polysaccharides, complex carbohydrates, sugars, gangliosides, lipids and phospholipids; portions thereof and combinations thereof. In the context of the present disclosure, the term “antigen” typically refers to a polypeptide or protein antigen at least eight amino acid residues in length, which may comprise one or more post-translational modifications.

The terms “polypeptide” and “protein” are used interchangeably to refer to a polymer of amino acid residues, and are not limited to a certain length unless otherwise specified. Polypeptides may include natural amino acid residues or a combination of natural and non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity (e.g., antigenicity).

The terms “isolated” and “purified” as used herein refers to a material that is removed from at least one component with which it is naturally associated (e.g., removed from its original environment). The term “isolated,” when used in reference to a recombinant protein, refers to a protein that has been removed from the culture medium of the host cell that produced the protein. In some embodiments, an isolated protein (e.g., SARS-COV-2 Spike protein) is at least 75%, 90%, 95%, 96%, 97%, 98% or 99% pure as determined by HPLC.

An “effective amount” or a “sufficient amount” of a substance is that amount sufficient to effect beneficial or desired results, including clinical results, and, as such, an “effective amount” depends upon the context in which it is being applied. In the context of administering a composition of the present disclosure comprising an mRNA encoding an antigen, an effective amount contains sufficient mRNA to stimulate an immune response (preferably a cellular immune response against the antigen).

In the present disclosure, the terms “individual” and “subject” refer to a mammals. “Mammals” include, but are not limited to, humans, non-human primates (e.g., monkeys), farm animals, sport animals, rodents (e.g., mice and rats) and pets (e.g., dogs and cats). In some preferred embodiments, the subject is a human subject.

The term “dose” as used herein in reference to a composition comprising a mRNA encoding an antigen refers to a measured portion of the taken by (administered to or received by) a subject at any one time. Administering a composition of the present disclosure to a subject in need thereof, comprises administering an effective amount of a composition comprising a mRNA encoding an antigen to stimulate an immune response to the antigen in the subject.

“Stimulation” of a response or parameter includes eliciting and/or enhancing that response or parameter when compared to otherwise same conditions except for a parameter of interest, or alternatively, as compared to another condition (e.g., increase in antigen-specific cytokine secretion after administration of a composition comprising or encoding the antigen as compared to administration of a control composition not comprising or encoding the antigen).

For example, “stimulation” of an immune response (e.g., Thl response) means an increase in the response. Depending upon the parameter measured, the increase may be from 2-fold to 200-fold or over, from 5-fold to 500-fold or over, from 10-fold to 1000-fold or over, or from 2, 5, 10, 50, or 100-fold to 200, 500, 1,000, 5,000, or 10,000-fold.

Conversely, “inhibition” of a response or parameter includes reducing and/or repressing that response or parameter when compared to otherwise same conditions except for a parameter of interest, or alternatively, as compared to another condition. For example, “inhibition” of an immune response (e.g., Th2 response) means a decrease in the response. Depending upon the parameter measured, the decrease may be from 2-fold to 200-fold, from 5-fold to 500-fold or over, from 10-fold to 1000-fold or over, or from 2, 5, 10, 50, or 100-fold to 200, 500, 1,000, 2,000, 5,000, or 10,000-fold.

The relative terms “higher” and “lower” refer to a measurable increase or decrease, respectively, in a response or parameter when compared to otherwise same conditions except for a parameter of interest, or alternatively, as compared to another condition. For instance, a “higher antibody titer” refers to an antigen-reactive antibody titer as a consequence of administration of a composition of the present disclosure comprising an mRNA encoding an antigen that is at least 2, 3, 4, 5, 6, 7, 8, 9, or 10-fold above an antigen-reactive antibody titer as a consequence of a control condition (e.g., administration of a comparator composition that does not comprise the mRNA or comprises a control mRNA that does not encode the antigen). Likewise, a “lower antibody titer” refers to an antigen-reactive antibody titer as a consequence of a control condition (e.g., administration of a comparator composition that does not comprise the mRNA or comprises a control mRNA that does not encode the antigen) that is at least 2, 3, 4, 5, 6, 7, 8, 9, or 10-fold below an antigen-reactive antibody titer as a consequence of administration of a composition of the present disclosure comprising an mRNA encoding an antigen.

As used herein the term “immunization” refers to a process that increases a mammalian subject's reaction to antigen and therefore improves its ability to resist or overcome infection and/or resist disease.

The term “vaccination” as used herein refers to the introduction of a vaccine into a body of a mammalian subject.

As used herein, “percent (%) amino acid sequence identity” and “percent identity” and “sequence identity” when used with respect to an amino acid sequence (reference polypeptide sequence) is defined as the percentage of amino acid residues in a candidate sequence (e.g., the subject antigen) that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

An amino acid substitution may include replacement of one amino acid in a polypeptide with another amino acid. Amino acid substitutions may be introduced into an antigen of interest and the products screened for a desired activity, e.g., increased stability and/or immunogenicity.

Amino acids generally can be grouped according to the following common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro; and
(6) aromatic: Trp, Tyr, Phe.

Conservative amino acid substitutions will involve exchanging a member of one of these classes with another member of the same class. Non-conservative amino acid substitutions will involve exchanging a member of one of these classes with a member of another class.

As used herein, the term “excipient” refers to a compound present in a composition comprising an active ingredient (e.g., mRNA encoding an antigen). Pharmaceutically acceptable excipients are inert pharmaceutical compounds, and may include for instance, solvents, bulking agents, buffering agents, tonicity adjusting agents, and preservatives (Pramanick et al., Pharma Times, 45:65-77, 2013). In some embodiments the compositions of the present disclosure comprise an excipient that functions as one or more of a solvent, a bulking agent, a buffering agent, and a tonicity adjusting agent (e.g., sodium chloride in saline may serve as both an aqueous vehicle and a tonicity adjusting agent).

### Optimized for Intradermal Delivery

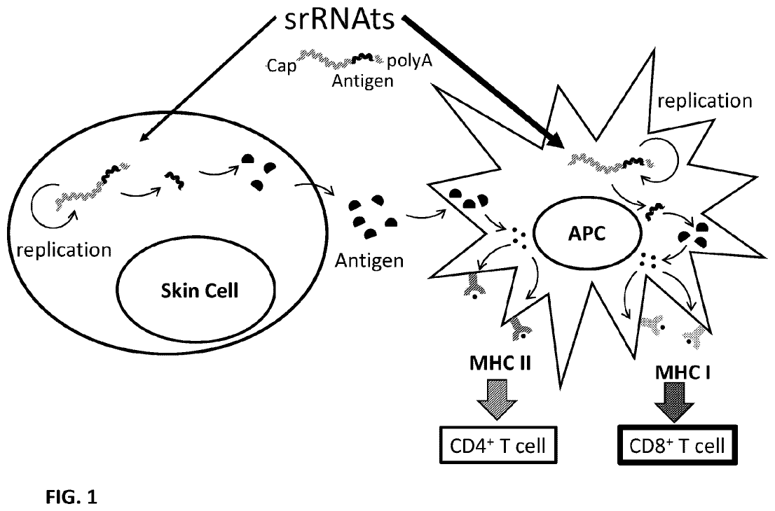

Intradermal vaccination results in long-lasting cellular immunity and increased immunogenicity [Hickling and Jones, 2009]. Human skin (epidermis and dermis) is rich in antigen-presenting cells (APCs), including Langerhans cells and dermal dendritic cells (DCs). Intradermal vaccination is known to be 5- to 10-times more effective than subcutaneous or intramuscular vaccination because it targets the APCs present in skin [Hickling and Jones, 2009], thereby activating the T cell immunity pathway for long-lasting immunity. By intradermal injection, srRNAts is predominantly taken up by skin APCs, wherein it replicates, produces antigen, digests the antigen into peptides, and presents the peptides to T cells (FIG. 1). The peptides presented through this pathway stimulates MHC-I-restricted CD8+ killer T cells. In an alternative pathway, APCs also take antigens produced by nearby skin cells. The peptides presented through this pathway stimulate MHC-II-restricted CD4+ Helper T cells, which helps B cells to produce neutralizing antibodies (nAb) to fight virus infection.

### Issues and Solutions for Intradermal Injection

Here are potential issues that we have identified and the solutions that the srRNAts platform offers.

(1) A key unrecognized hurdle for the application of srRNA as an intradermal vaccine platform is that both mRNA and srRNA do not express antigen well at skin temperature [PCT/US20/67506]. Unintuitively, the temperature of the human skin is lower (about 30-35° C.) than human core body temperature (about 37° C.); this means that vectors and platforms developed at 37° C. are not optimal for intradermal injection. One innovation of the srRNAts platform is that it expresses antigen strongly at skin temperature [PCT/US20/67506]. Furthermore, this temperature-control also minimizes the safety risk caused by unintended systemic distribution of srRNAts because srRNAts becomes inactivated once its temperature increases above its permissive threshold (when it moves closer to the core of the body). In other words, the srRNAts platform expresses antigen the best for intradermal injection compared to mRNA and srRNA, and it additionally has safety features: the vector's ability to spread and become produced in other areas of a subject's body is limited or inactivated.

(2) Another challenge for intradermal vaccination is the lack of suitable additives. Because adjuvants such as aluminum-salt and oil-in-water are too reactogenic locally when delivered by the intradermal route, no adjuvant has been incorporated into clinically approved intradermal vaccines, resulting in lower immunogenicity [Hickling and Jones, 2009]. Lipid Nanoparticles (LNPs) used for mRNA and srRNA vaccines, which are administered intramuscularly, are also oil-in-water, which may cause skin reactogenicity and increase risk of allergic reactions to LNP components such as PEG. The c-srRNA platform is a solution to this problem since it is injected as naked c-srRNA (no LNPs, no adjuvants). First, self-replication of RNAs inside cells, especially APCs, induces the strong innate immunity, which substitutes the major functions of adjuvants. Second, data in the literature and obtained during development of the present disclosure demonstrates that, specifically for intradermal injection, naked mRNA/srRNA is equally efficient to produce an antigen compared to electroporation of mRNA/srRNA [Johansson et al, 2012] and mRNA/srRNAs combined with LNPs [Golombek et al., 2018].

(3) A third challenge is the limited number of precedents for intradermal vaccines. Only the BCG vaccine has been administered intradermally on a routine basis, and currently available COVID-19 vaccines are all administered intramuscularly. One way we lower the hurdle for adopting intradermal injection is by using specialized devices such as the MicronJet600 (NanoPass) and Immucise (Terumo), which are now available to enable easy, consistent intradermal injection. These devices are also good candidates for large-scale production and deployment. However, due to a relatively high cost of these special devices, an intradermal injection by the Mantoux technique using a standard needle and syringe is also an option.

### Design of Suitable Antigens

The cellular immunity-focused approach allowed for the reconsideration of all the proteins encoded on viral genomes as antigen candidates, as humoral immunity, i.e., the induction of neutralizing antibodies, is not the primary consideration.

When selecting an antigen that would provide broader protection against SARS-CoV-1, SARS-COV-2, MERS-COV, and their variants, the Nucleoprotein (N) was determined to be the most suitable, because (1) N is the most abundant protein, followed by Membrane (M) and Spike (S) in viral particles [Finkel et al., 2021], (2) N is overall the most conserved protein among the above indicated Betacoronaviruses [Grifoni et al., 2020], and (3) epitopes for B and T cells are the most abundant in S and N [Grifoni et al., 2020]. This is consistent with the earlier proposal that N is the best antigen for the vaccine [Dutta et al., 2020]. Notably, a recent report clearly demonstrated that a vaccine using N alone as an antigen can provide an S-independent protective immunity in both hamster and mouse [Matchett et al., 2021]. Although disease enhancement was observed for N vaccines, as well as S vaccines previously [Lambert et al., 2020], these data were obtained by using different vectors with unfavorable Th2>Th1 profiles.

An exemplary vaccine candidate, srRNA1ts2-G5005, was designed to express the N protein of SARS-COV-2 (SARS2-N). However, MERS-N forms a distinct group and shows only 48% identity [Tilocca et al., 2020]. With this in mind, a further exemplary vaccine candidate, srRNA1ts2-G5006, was designed to express a fusion protein of SARS2-N and MERS-N. The G5005 and G5006 antigens are shown schematically in FIG. 2. srRNAlts2-G5005 is suitable for induction of immune responses against SARS-COV-1, SARS-COV-2, and their variants. In contrast, srRNA1ts2-G5006 is suitable for induction of a pan-coronavirus immune response (e.g., against SARS-COV-1, SARS-COV-2, MERS-COV, and their variants).

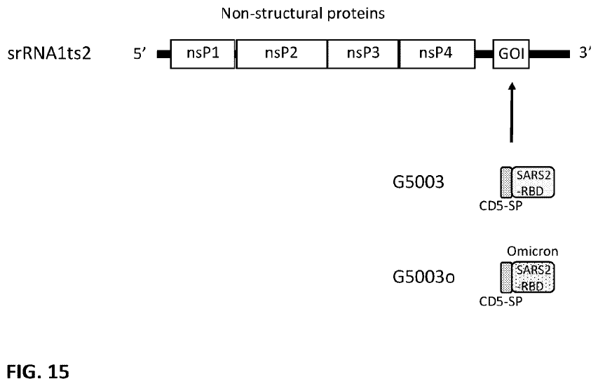

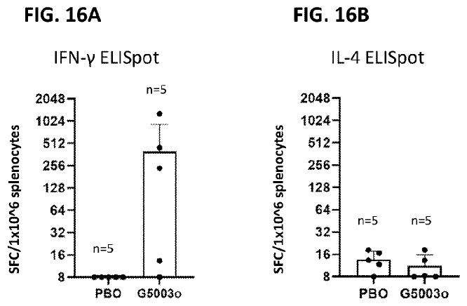

To address the emergence of a variant (mutated) form of SARS-COV-2 virus, c-srRNA encoding the RBD of SARS-COV-2 omicron variant (G50030) was generated and intradermally administered to C57BL/6 mice (Example 8 and FIG. 15). Cellular immunity was assessed 14 days after the vaccination. The results clearly demonstrate that c-srRNA can induce omicron variant-specific cellular immunity, when the open reading frame of the receptor binding domain (RBD) of the omicron variant is included in the c-srRNA. Importantly, c-srRNA encoding the G50030 antigen was found to induce a Th1-biased response as shown in FIG. 16A-16B [Th1 (INF-γ)>Th2 (IL-4], which is favored for vaccines.

### Inclusion in Prime-Boost Immunization Regimens

One of the unique features of intradermally administered c-srRNA vaccine is its ability to induce cellular immunity without apparent induction of humoral immunity (i.e., antibodies). As determined during development of the present disclosure, c-srRNA vaccines are able to prime a humoral immune response to a subsequently encountered protein antigen. In brief, mice were first treated with c-srRNA encoding an antigen (i.e., RBD of SARS-COV-2 Wuhan strain) and were subsequently treated with an adjuvanted variant RBD protein (i.e., RBD of SARS-COV-2 Delta variant) as described in Example 9 and shown in FIG. 17A.

Cellular immunity, assessed by measuring the presence of antigen-specific IFN-γ-secreting T cells, was already induced by day 14 post-primary vaccination (prime) as shown in FIG. 17B. Antigen-specific antibody was not detected at this time. After treatment with the adjuvanted protein antigen, antibodies were induced as early as day 7 post-secondary vaccination (boost) as shown in FIG. 17C. This early induction of antibodies is consistent with a secondary immune response, indicating that c-srRNA already primed humoral immunity. Importantly, the antibodies induced by the protein antigen boost were able to neutralize the viral variant, which has a distinct RBD sequence from the RBD antigen encoded by the c-srRNA vaccine. This surprising finding indicates that the c-srRNA vaccine can induce a protective immune response against a pathogen with an antigen sequence that differs from the antigen sequence encoded by the c-srRNA vaccine. Thus, the c-srRNA vaccines are expected to induce broadly reactive immune responses, which are critical for providing protection against variant pathogens.

Subunit vaccines against pathogens generally do not provide the long-lasting humoral immunity (i.e., pathogen-specific antibodies), and therefore one or more booster vaccines are required. As determined during development of the present disclosure, c-srRNA vaccines are suitable for use as a booster vaccine, when an adjuvanted protein is administered as a prime vaccine. In brief, mice were first treated with adjuvanted protein (i.e., RBD of SARS-CoV-2 Wuhan strain) and were subsequently treated with a placebo (PBO: buffer only), c-srRNA encoding G5003 antigen (Wuhan RBD), c-srRNA encoding G50030 antigen (Omicron RBD), or the adjuvanted protein antigen (Wuhan RBD) as described in Example 10 and shown in FIG. 18A.

As shown in FIG. 17C, c-srRNA vaccine alone does not induce humoral immunity in the form of a neutralizing antibody response (see, PBO day 7). However, when humoral immunity is primed by the adjuvanted protein (as a model for primary vaccination), the c-srRNA booster vaccine is able to induce both antigen-specific cytokine responses (FIG. 18B-18C) and antigen-specific antibody responses (FIG. 19). It is worth noting that in the current experimental condition, a single dose of adjuvanted protein did not induce RBD-specific antibodies. Apparently, cellular immunity induced by c-srRNA is capable of stimulating antibody production to an earlier encountered protein antigen. This observation is indicative of important interactions occurring between cellular and humoral immune responses.

### Elimination of Antigen-Expressing Cells in Vivo

c-srRNA vaccines are able to induce strong cellular immune responses (i.e., antigen-specific CD8+ cytotoxic T lymphocytes and CD4+ helper T lymphocytes). Antigen-specific CD8+ CTL lyse cells in which the antigen is expressed. Antigen recognition by CD8+ CTL is based on presentation of short peptide fragments (T cell epitopes) by MHC class I molecules, and thus, the antigen does not have to be expressed on the surface of target cells. For a vaccine directed against a pathogen, the vaccine is expected to lyse cells infected with the pathogen. For a vaccine directed against a cancer, the vaccine is expected to lyse cancer cells.

A c-srRNA vaccine encoding a fusion protein of SARS-COV-2 nucleoprotein and MERS-COV nucleoprotein (called SMN protein or G5006) as an antigen was produced. In order to model cells infected with a virus, a 4T1 breast cancer cell line derived from BALB/c mouse and known as a model for a triple-negative stage IV human breast cancer was selected. When injected into BALB/c mice, the 4T1 cells grow rapidly and form tumors. This syngeneic mouse model was used to mimic the rapid increase of infected cells. The 4T1 cells expressing the SMN protein (named 4T1-SMN) was established by transfecting a plasmid vector encoding an SMN protein under the CMV promoter, so that the protein is constitutively expressed in 4T1 cells. The fusion protein is the same as G5006 except that the CD5 signal peptide was removed from the N-terminus of the SMN protein expressed in 4T1 cells.

BALB/c mice were vaccinated with c-srRNA-G5006, and the induction of cellular immunity was demonstrated by the presence of T-cells that responded to both SARS-CoV-2 nucleoprotein (FIG. 20A-20B) and MERS-COV nucleoprotein (FIG. 20C-20D). Subsequently, 4T1-SMN cells were injected into the BALB/c mice vaccinated with c-srRNA-G5006 on day 24 (24 days post-vaccination). As expected, 4T1-SMN cells grew rapidly in mice that received a placebo (no vaccine group). In contrast, the growth of 4T1-SMN tumors was suppressed in the c-srRNA-G5006 vaccinated mice. In two mice that received 25 μg of the c-srRNA-G5006 vaccine, while the tumors initially grew, the mice eventually became tumor-free and survived long after the recipients of the placebo had died. Furthermore, even after the second round of injection of 4T1-SMN tumors on day 143 after vaccination, no tumors grew, and the mice remained alive and tumor-free for the duration of the study (FIG. 21). This result suggests that a c-srRNA vaccine encoding the G5006 antigen (i.e., SMN protein) can induce a protective immune response by elimination of cells infected with SARS-COV-2 or MERS-COV.

### Pancoronavirus Booster Vaccine

For infectious diseases, such as COVID-19, World Health Organization guidelines require a licensed vaccine to be capable of inducing neutralizing antibodies (nAb). This requirement makes sense since nAb can prevent cells from becoming infected, and thus nAb can efficiently control the spread of infection. However, nAb levels generally decline rapidly, and therefore booster vaccines are needed periodically (e.g., once or twice a year) after completion of a primary vaccination series (1st and 2nd vaccinations) to maintain adequate nAb levels. The high mutation rate of SARS-COV-2, particularly within the RBD of the Spike protein, which is a target for nAb, is a major concern associated with the use of first generation COVID-19 vaccines that typically target SARS-COV-2 Spike protein.

To address these issues, a new booster vaccine was developed, c-srRNA-G5006d, which encodes a fusion protein comprising the CD5 signal peptide, Spike-RBD of SARS-COV-2, nucleoprotein of SARS-COV-2, nucleoprotein of MERS-COV, and Spike-RBD of MERS-COV (Example 12 and FIG. 22). The amino acid sequence of the pancoronavirus antigen (G5006d) is set forth as SEQ ID NO:27, and the nucleotide sequence of its open reading frame is set forth as SEQ ID NO:26. The order of each sequence segment (RBD of SARS-COV-2; a nucleoprotein of SARS-COV-2; a nucleoprotein of MERS-COV; RBD of MERS-COV) of the fusion protein can be altered, and the amino acid sequences of each segment do not have to be 100% identical to the exemplary sequences provided herein.

The c-srRNA-G5006d vaccine is intended to be used as a booster vaccine, after a primary vaccine series (1st vaccination or 1st and 2nd vaccinations) targeted to the Spike antigen or fragment thereof (RBD) has been received. However, the c-srRNA-G5006d vaccine could also be used as part of a primary vaccine series.

The c-srRNA-G5006d vaccine boosts nAb levels and provides cellular immunity against betacoronaviruses that infect humans. Cellular immunity is important for providing long-lasting protection from severe illness, hospitalization, and death.

As described in Example 10, a c-srRNA vaccine encoding Spike-RBD can increase the level of antibodies or nAb against Spike-RBD, when it was used as a booster vaccine, following administration of a vaccine that can prime or induce humoral immunity.

The c-srRNA-G5006d encodes both Spike-RBD protein of SARS-COV-2 and Spike-RBD protein of MERS-COV. Therefore, c-srRNA-G5006d can be used as a booster vaccine for both SARS-COV-2 and MERS-COV.

Spike proteins of SARS-COV-2 and SARS-COV are similar (about 76% identity) (Grifoni et al., 2020). Therefore, c-srRNA-G5006d is effective as a booster for SARS-COV-2, SARS-COV, and their variants. On the other hand, Spike proteins of SARS-COV-2 and MERS-CoV are different (about 35% identity) (Grifoni et al., 2020). However, c-srRNA-G5006d also encodes a Spike-RBD of MERS-COV. Therefore, c-srRNA-G5006d is effective as a booster for MERS-COV and its variants. Taken together, c-srRNA-G5006d is effective as a booster for SARS-COV-2, SARS-COV, MERS-COV, and their variants.

The c-srRNA-G5006d also encodes nucleoproteins of SARS-COV-2 and MERS-CoV. Therefore, c-srRNA-G5006d is able to induce strong cellular immunity against SARS-CoV-2 and MERS-COV. Nucleoproteins of SARS-COV-2 and SARS-COV are very similar to each other (about 90% identity) (Grifoni et al., 2020). Therefore, c-srRNA-G5006d provides strong cellular immunity against SARS-COV-2, SARS-COV, and their variants. In contrast, nucleoproteins of SARS-COV-2 and MERS-COV are different (about 48% identity) (Grifoni et al., 2020). However, c-srRNA-G5006d also encodes a nucleoprotein of MERS-COV. Therefore, c-srRNA-G5006d is contemplated to provide strong cellular immunity against MERS-COV and its variants. Taken together, c-srRNA-G5006dinduces a potent immune response against SARS-CoV-2, SARS-COV, MERS-COV, and their variants.

As described in Examples 9 and 10, c-srRNA vaccine has a remarkable mode of action. That is, the encoded antigens do not appear to directly stimulate B cells, and thus, consideration of three-dimensional structure of the encoded antigens is not required. This differs from traditional vaccine that are designed to directly stimulate the B cells to produce antibodies against conformational epitopes (three-dimensional structures of antigens). This is why it is appropriate to use a fusion protein for a c-srRNA vaccine, whereas use of a fusion protein for a traditional subunit vaccine is complicated by the fact that the natural three-dimensional structure of each antigen may be disrupted when expressed as a fusion protein. The c-srRNA booster vaccine stimulates antibody production through the activation of CD4+ helper T cells, and thus, it relies on short peptide epitopes (˜15 mer). Therefore, it is possible to simply put together two or more different antigens into a single fusion protein for an antigen encoded by a c-srRNA vaccine, while this mechanism may be problematic for design of a subunit vaccine.

The fact that c-srRNA relies on short peptide epitopes for induction of cellular and humoral immune responses also provides advantages for more broadly reactive vaccines that elicit protection against variant pathogens. Many T cell epitopes are present in a single protein, and thus, it is less likely that any single mutation will cause the loss of immunogenicity. On the other hand, traditional subunit vaccines rely on the three-dimensional structure of a protein antigen, and thus, even a single mutation may alter the conformation of the protein, which may lead to the loss of immunogenicity.

As shown in FIGS. 23A-23B, c-srRNA-G5006d can stimulate cellular immunity against all proteins encoded by this vaccine: Spike-RBD of SARS-COV-2, Nucleoprotein of SARS-COV-2, Nucleoprotein of MERS-COV, and Spike-RBD of MERS-COV.

### Pan-influenza Booster Vaccine

As determined during development of the present disclosure (see, e.g.,

Example 6), a fusion protein comprising nucleoproteins from representative Influenza A and Influenza B strains was able to induce a strong, antigen-specific cellular immune response when the fusion protein was expressed from an intradermally-injected, temperature-controllable, self- replicating RNA. Protection is generally considered to be mainly mediated by neutralizing antibodies against hemagglutinin (HA), one of the surface proteins of influenza viruses. Therefore, FDA-approved influenza vaccines include HA as an antigen, alone or in combination with other influenza antigens. Since a c-srRNA-based booster vaccine requires only CD4+ T cell epitopes on the HA protein to enhance Ab production, the three-dimensional structure of the HA protein does not need to be considered. It is known that only some parts of the HA protein of the H1N1 influenza virus can function as CD4+ T cell epitopes (Knowlden et al., Pathogens. 8(4):220, 2019). B cell epitopes and CD4+ T cell epitopes in both influenza A and influenza B have been identified (Terajima et al. Virol J, 10:244, 2013). Sequences of HA proteins of representative H1N1 influenza viruses were aligned (Darricarrère et al., J Virol, 92(22):e01349-18, 2018) and regions with well-conserved sequences were identified. Based on these considerations, an HA protein fragment (residues 316-456) of Influenza A virus (A/New Caledonia/20/1999(H1N1)) [GenBank Accession No. EU103824] and an HA protein fragment (residues 332-474) of Influenza B virus (B/Florida/4/2006) [GenBank Accession No. CY033876] were selected. The nucleoproteins from Influenza A and Influenza B, which are already described in Example 6 and denoted as the G5010 antigen were also included.

FIG. 24 shows the design of pan-influenza booster vaccine. The c-srRNA-G5012 encodes a fusion protein (G5012) comprising the signal peptide of CD5 (residues 1-24), a part of the hemagglutinin (HA) of the Influenza A, nucleoprotein of Influenza A, nucleoprotein of Influenza B, and a part of the hemagglutinin (HA) of the Influenza B. The amino acid sequence of the pan-influenza virus antigen (G5012) is set forth as SEQ ID NO:29, and the nucleotide sequence of its open reading frame is set forth as SEQ ID NO:28. The order of each sequence segment (a part of HA of Influenza A; a nucleoprotein Influenza A; a nucleoprotein of Influenza B; a part of HA of Influenza B) of the fusion protein can be altered, and the amino acid sequences of each segment do not have to be 100% identical to the exemplary sequences provided herein.

This c-srRNA-G5012 Influenza vaccine boosts nAb levels through the enhancement of HA-specific CD4+ helper T cells. It also provides cellular immunity against essentially all Influenza viruses through the evolutionary conserved nucleoproteins. The cellular immunity is known to provide a long-lasting protection from severe illness, hospitalization, and death.

### Chitosan-Enhancement of Gene Expression in vivo

An RNase inhibitor (a protein purified from human placenta) slightly enhances the immunogenicity against an antigen encoded on c-srRNA, most likely by enhancing expression of the antigen from the c-srRNA in vivo when intradermally injected into mice (see e.g., FIG. 25C of WO 2021/138447 A1). The RNase inhibitor may protect c-srRNA from RNase-mediated degradation in vivo. However, it is desirable to find an alternative agent that can enhance expression of a gene of interest (GOI) in vivo for therapeutics purposes, as it is difficult to use a protein-based RNase inhibitor as an excipient in injectable products.

A low molecular weight chitosan (molecular weight ˜ 6 kDa) was shown to inhibit the activity of RNase with the inhibition constants in the range of 30-220 nM (Yakovlev et al., Biochem Biophys Res Commun, 357(3):584-8, 2007). Although this has been shown only in vitro and also for artificially made poly nucleotides such as Poly(A)/Poly(U), whether chitosan oligosaccharides can enhance the expression of GOI from c-srRNA needed to be tested in vivo by intradermally injecting the c-srRNA in mice. As shown in Example 14, two different chitosan oligomers were tested: chitosan oligomer (molecular weight ≤5 kDa, ≥75% deacetylated: Heppe Medical Chitosan GmbH: Product No. 44009), and chitosan oligosaccharide lactate (molecular weight about 5 kDa, >90% deacetylated: Sigma-Aldrich: Product No. 523682). Surprisingly, even a very low level of chitosan oligomers, as low as 0.001 μg/mL (about 0.2 nM: about 1/100 of the inhibition constant discovered by Yakovlev et al., supra, 2007) was found to be able to enhance the expression of luciferase encoded on c-srRNA by ˜10-fold (FIG. 25). Similar enhancement of the GOI expression was achieved by chitosan oligomers for up to 0.5 μg/mL and by chitosan oligosaccharide lactate at 0.1 μg/mL.

Chitosan has been used as a nucleotide (DNA and RNA) delivery vector, as it can form complexes or nanoparticles (reviewed in Buschmann et al., Adv Drug Deliv Rev, 65(9): 1234-70, 2013; and Cao et al., Drugs, 17:381, 2019). However, it is worth noting that the enhancement of the GOI expression by chitosan oligomers is unlikely to be mediated by the nanoparticle or the complex formation of c-srRNA and chitosan oligomers. First, such a low concentration of chitosan oligomers does not allow the complex formation with RNA. Second, chitosan oligomers are added to c-srRNA immediately before the intradermal injection, and thus, there is not sufficient time to form the complex.

As the chitosan oligomers enhance expression of the GOI in vivo at much lower concentrations compared to the effective concentration as an RNase inhibitor in vitro (Yakovlev et al., supra, 2007), it is conceivable that this enhanced GOI expression by chitosan oligomers may not be mediated by its RNase inhibition mechanism. For example, chitosan oligomers may facilitate the incorporation of c-srRNA into cells, and thereby may enhance the expression of GOI from c-srRNA. Nonetheless, this surprising discovery should provide an effective means to enhance the in vivo therapeutic expression of GOI encoded on c-srRNA.

### ENUMERATED EMBODIMENTS

1. A composition for stimulating an immune response against a coronavirus in a mammalian subject, comprising an excipient, and a messenger RNA (mRNA) comprising an open reading frame (ORF) encoding a fusion protein, wherein the ORF comprises from 5′ to 3′:
(i) a nucleotide sequence encoding a mammalian signal peptide; and
(ii) a nucleotide sequence encoding a coronavirus nucleocapsid protein.

2 The composition of embodiment 1, wherein the coronavirus is a betacoronavirus, optionally wherein the betacoronavirus is a human betacoronavirus.

3 The composition of embodiment 2, wherein the betacoronavirus comprises a severe acute respiratory syndrome coronavirus-2 (SARS-COV-2), a severe acute respiratory syndrome coronavirus-1 (SARS-COV-1), a middle east respiratory syndrome-related coronavirus (MERS-COV), or a combination thereof.

4. The composition of embodiment 3, wherein the betacoronavirus comprises a severe acute respiratory syndrome coronavirus-2 (SARS-COV-2).

5. The composition of embodiment 4, wherein the coronavirus nucleocapsid protein comprises a first nucleocapsid protein and a second nucleocapsid protein, wherein the first nucleocapsid protein is a SARS-COV-2 nucleocapsid protein of a first variant from a first clade, and the second nucleocapsid protein is a SARS-COV-2 nucleocapsid protein of a second variant from a second clade, and wherein the first clade and the second clade are different clades as defined by one or more of the World Health Organization, Pango, GISAID, and Nextstrain.

6. A composition for stimulating an immune response against a coronavirus in a mammalian subject, comprising an excipient, and a messenger RNA (mRNA) comprising an open reading frame (ORF) encoding a fusion protein, wherein the ORF comprises from 5′ to 3′:
(i) a nucleotide sequence encoding a mammalian signal peptide; and
(ii) a nucleotide sequence encoding two or more coronavirus nucleocapsid proteins.

7. The composition of embodiment 6, wherein the coronavirus is a betacoronavirus, optionally wherein the betacoronavirus is a human betacoronavirus.

8. The composition of embodiment 7, wherein the betacoronavirus comprises a severe acute respiratory syndrome coronavirus-2 (SARS-COV-2), a severe acute respiratory syndrome coronavirus-1 (SARS-COV-1), a middle east respiratory syndrome-related coronavirus (MERS-COV), or a combination thereof.

9. The composition of embodiment 8, wherein the betacoronavirus comprises a severe acute respiratory syndrome coronavirus-2 (SARS-COV-2).

10. The composition of embodiment 9, wherein the two or more coronavirus nucleocapsid proteins comprise a SARS-COV-2 nucleocapsid protein and a MERS nucleocapsid protein.

11. The composition of embodiment 9, wherein the two or more coronavirus nucleocapsid proteins comprise a SARS-COV-2 nucleocapsid protein, a SARS-COV-1 nucleocapsid protein, and a MERS nucleocapsid protein.

12. The composition of any one of embodiments 6-11, wherein the two or more coronavirus nucleocapsid proteins are separated by a linker of from one to ten residues in length.

13. The composition of any one of embodiments 1-12, wherein the mammalian signal peptide is a signal peptide of a surface protein expressed in mammalian antigen presenting cells.

14. The composition of embodiment 13, wherein the mammalian signal peptide is a CD5 signal peptide and the amino acid sequence of the CD5 signal peptide comprises SEQ ID NO:8, or the amino acid sequence at least 90% or 95% identical to SEQ ID NO:8.

15. The composition of any one of embodiments 1-14, wherein the amino acid sequence of the nucleocapsid protein comprises residues 2-419 of SEQ ID NO:5, or the amino acid sequence at least 75%, 85%, 90%, 95%, 96%, 97%, 98% or 99% identical to residues 2-419 of SEQ ID NO:5.

16. The composition of any one of embodiments 1-14, wherein the amino acid sequence of the fusion protein comprises SEQ ID NO:6, or the amino acid sequence at least 75%, 85%, 90%, 95%, 96%, 97%, 98% or 99% identical to SEQ ID NO:6.

17. The composition of any one of embodiments 6-14, wherein the amino acid sequence of the fusion protein comprises SEQ ID NO:7, or the amino acid sequence at least 75%, 85%, 90%, 95%, 96%, 97%, 98% or 99% identical to SEQ ID NO:7.

18. The composition of embodiment 16, wherein the open reading frame comprises the nucleotide sequence of SEQ ID NO:2.

19. The composition of embodiment 17, wherein the open reading frame comprises the nucleotide sequence of SEQ ID NO:3 or SEQ ID NO:4.

20. The composition of any one of embodiments 1-14, wherein the amino acid sequence of the fusion protein comprises residues 2-413 of SEQ ID NO:9, or the amino acid sequence at least 75%, 85%, 90%, 95%, 96%, 97%, 98% or 99% identical to residues 2-413 of SEQ ID NO:9.

21. The composition of any one of embodiments 1-14, wherein the amino acid sequence of the fusion protein comprises residues 2-422 of SEQ ID NO:10, or the amino acid sequence at least 75%, 85%, 90%, 95%, 96%, 97%, 98% or 99% identical to residues 2-422 of SEQ ID NO:10.

22. The composition of any one of embodiments 1-21, wherein the composition does not comprise liposomes or lipid nanoparticles.

23. The composition of any one of embodiments 1-22, wherein the mRNA is a self-replicating mRNA.

24. The composition of embodiment 23, wherein the self-replicating RNA comprises an Alphavirus replicon lacking a viral structural protein coding region.

25. The composition of embodiment 24, wherein the Alphavirus is selected from the group consisting of a Venezuelan equine encephalitis virus, a Sindbis virus, and a Semliki Forrest virus.

26. The composition of embodiment 25, wherein the Alphavirus is a Venezuelan equine encephalitis virus.

27. The composition of any one of embodiments 23-26, wherein the Alphavirus replicon comprises a nonstructural protein coding region with an insertion of 12-18 nucleotides resulting in expression of a nonstructural Protein 2 (nsP2) comprising from 4 to 6 additional amino acids between beta sheet 4 and beta sheet 6 of the nsP2.

28. The composition of any one of embodiments 1-27, wherein the self-replicating mRNA is a temperature-sensitive agent (ts-agent) that is capable of expressing the fusion at a permissive temperature but not at a non-permissive temperature.

29. The composition of embodiment 28, wherein the permissive temperature is from 31° C. to 35° C. and the non-permissive temperature is at least 37° C. +0.5° C.

30. A method for stimulating an immune response against a coronavirus in a mammalian subject, comprising administering the composition of any one of embodiments 1-29 to a mammalian subject so as to stimulate an immune response against the coronavirus nucleocapsid protein in the mammalian subject

31. The method of embodiment 30, wherein the composition is administered intradermally.

32. The method of embodiment 30 or embodiment 31, wherein the immune response comprises a coronavirus-reactive cellular immune response.

33. The method of embodiment 32, wherein the immune response further comprises a coronavirus-reactive humoral immune response.

34. The method of any one of embodiments 30-33, wherein the mammalian subject is a human subject.

35. A kit comprising:
the composition of any one of embodiments 1-29 or any one of embodiments 37-62; and
a device for intradermal delivery of the composition to a mammalian subject.

36. The kit of embodiment 35, wherein the device comprises a syringe and a needle.

37. A composition for stimulating an immune response against two or more viruses in a mammalian subject, comprising an excipient, and a messenger RNA (mRNA) comprising an open reading frame (ORF) encoding a fusion protein, wherein the ORF comprises from 5′ to 3′:
(i) a nucleotide sequence encoding a mammalian signal peptide; and
(ii) a nucleotide sequence encoding a first nucleocapsid protein of a first virus and a second nucleocapsid protein of a second virus.

38. The composition of embodiment 37, wherein the first and second viruses are capable of causing disease upon infection of a human subject.

39. The composition of embodiment 38, wherein the first and second viruses are different variants, subtypes or lineages of the same species.

40. The composition of embodiment 38, wherein the first and second viruses are different species of the same genus.

41. The composition of embodiment 40, wherein the first and second viruses are both members of the betacoronavirus genus.

42. The composition of embodiment 41, wherein the first and second viruses comprise a severe acute respiratory syndrome coronavirus-2 (SARS-COV-2) and a middle east respiratory syndrome-related coronavirus (MERS-COV).

43. The composition of embodiment 38, wherein the first and second viruses are members of different families, orders, classes, or phyla of the same kingdom.

44. The composition of embodiment 43, wherein the first and second viruses are both members of the orthomyxoviridae family.

45. The composition of embodiment 44, wherein the first and second viruses comprise an influenza A virus and an influenza B virus.

46. The composition of embodiment 45, wherein the amino acid sequence of the fusion protein comprises SEQ ID NO:16, or the amino acid sequence at least 75%, 85%, 90%, 95%, 96%, 97%, 98% or 99% identical to SEQ ID NO:16.

47. The composition of embodiment 38, wherein the first and second viruses are both members of the orthornavirae kingdom, optionally wherein the first and second viruses comprise: (a) a severe acute respiratory syndrome coronavirus-2 (SARS-COV-2), a severe acute respiratory syndrome coronavirus-1 (SARS-COV-1), or a middle east respiratory syndrome-related coronavirus (MERS-COV); and (b) an influenza A virus or an influenza B virus.

48. The composition of embodiment 40, wherein the first and second viruses are both members of the ebolavirus genus, optionally wherein the first and second viruses are selected from the group consisting of Zaire ebolavirus, Sudan ebolavirus, Bundibugyo ebolavirus, and Taï Forest ebolavirus.

49. The composition of embodiment 48, wherein the nucleotide sequence further encodes a third nucleocapsid protein of a third virus and a fourth nucleocapsid protein of a fourth virus, and the first, second, third and fourth viruses are Zaire ebolavirus, Sudan ebolavirus, Bundibugyo ebolavirus, and Taï Forest ebolavirus.

50. The composition of embodiment 49, wherein the amino acid sequence of the fusion protein comprises SEQ ID NO:22, or the amino acid sequence at least 75%, 85%, 90%, 95%, 96%, 97%, 98% or 99% identical to SEQ ID NO:22.

51. The composition of embodiment 49, wherein the nucleotide sequence (ii) encodes a shared portion of the first nucleocapsid protein of the first virus for stimulating an immune response against all of the first, second, third and fourth viruses.

52. The composition of embodiment 51, wherein the nucleotide sequence (ii) encodes an individual portion of each of the first, second, third and fourth nucleocapsid proteins for stimulating an immune response against all of the first, second, third and fourth viruses.

53. The composition of embodiment 52, wherein the nucleotide sequence (ii) encodes a fragment of the individual portion of the second nucleocapsid protein of the second virus for stimulating an immune response against the second and third viruses.

54. The composition of embodiment 37, wherein the nucleotide sequence (ii) encodes a shared portion of the first nucleocapsid protein of the first virus for stimulating an immune response against both the first and second viruses.

55. The composition of embodiment 54, wherein the nucleotide sequence (ii) encodes an individual portion of each of the first and second nucleocapsid proteins for stimulating an immune response against both the first and second viruses.

56. The composition of any one of embodiments 37-48, wherein the nucleotide sequence of (ii) further encodes at least one further nucleocapsid protein of at least one further virus, and wherein the at least one further virus is different from the first and second viruses.

57. The composition of any one of embodiments 37-56, wherein the first and second, or the first, second, and further nucleocapsid proteins are separated by a linker of from one to ten residues in length.

58. The composition of any one of embodiments 37-57, wherein the mammalian signal peptide is a signal peptide of a surface protein expressed in mammalian antigen presenting cells.

59. The composition of any one of embodiments 37-58, wherein the mRNA is a self-replicating mRNA.

60. The composition of embodiment 59, wherein the self-replicating mRNA is a temperature-sensitive agent (ts-agent) that is capable of expressing the fusion protein a permissive temperature but not at a non-permissive temperature.

61. The composition of embodiment 60, wherein the permissive temperature is from 31° C. to 35° C. and the non-permissive temperature is at least 37° C.±0.5° C.

62. The composition of any one of embodiments 1-29 or any one of embodiments 37-61, wherein the composition further comprises chitosan.

63. A method for stimulating an immune response against two or more viruses in a mammalian subject, comprising administering the composition of any one of embodiments 37-62 to a mammalian subject to stimulate an immune response against the nucleocapsid proteins of the two or more viruses in the mammalian subject

64. The method of embodiment 63, wherein the composition is administered intradermally.

65. The method of embodiment 63 or embodiment 64, wherein the immune response comprises a cellular immune response reactive with the two or more viruses.

66. The method of embodiment 65, wherein the cellular immune response comprises a nucleocapsid protein-specific helper T lymphocyte (Th) response comprising nucleocapsid protein-specific cytokine secretion.

67. The method of embodiment 66, wherein nucleocapsid protein-specific cytokine secretion comprises secretion of one or both of interferon-gamma and interleukin-4.

68. The method of embodiment 65, wherein the cellular immune response comprises a nucleocapsid protein-specific cytotoxic T lymphocyte (CTL) response.

69. The method of any one of embodiments 65-68, wherein the immune response further comprises a humoral immune response reactive with the two or more viruses.

70. The method of any one of embodiments 63-69, wherein the mammalian subject is a human subject.

71. A composition for stimulating an immune response against a virus in a mammalian subject, comprising an excipient, and a messenger RNA (mRNA) comprising an open reading frame (ORF) encoding a fusion protein, wherein the ORF comprises from 5′ to 3′:
(i) a nucleotide sequence encoding a mammalian signal peptide;
(ii) a nucleotide sequence encoding a first viral antigen or fragment thereof of a first virus; and
(iii) a nucleotide sequence encoding a second viral antigen or fragment thereof of the first virus or a second virus,
wherein the first viral antigen is a nucleocapsid protein and the second viral antigen is a surface protein, or the first viral antigen is a surface protein and the second viral antigen is a nucleocapsid protein.

72. A composition for stimulating an immune response against two or more viruses in a mammalian subject, comprising an excipient, and a messenger RNA (mRNA) comprising an open reading frame (ORF) encoding a fusion protein, wherein the ORF comprises from 5′ to 3′:
(i) a nucleotide sequence encoding a mammalian signal peptide;
(ii) a nucleotide sequence encoding a first viral antigen or fragment thereof of a first virus;
(iii) a nucleotide sequence encoding a second viral antigen or fragment thereof of the first virus;
(iv) a nucleotide sequence encoding a third viral antigen or fragment thereof of a second virus;
(iii) a nucleotide sequence encoding a fourth viral antigen or fragment thereof of the second virus,
wherein the first viral antigen is a first nucleocapsid protein and the second viral antigen is a first surface protein, or the first viral antigen is a first surface protein and the second viral antigen is a first nucleocapsid protein, and
wherein the third viral antigen is a second nucleocapsid protein and the fourth viral antigen is a second surface protein, or the third viral antigen is a second surface protein and the fourth viral antigen is a second nucleocapsid protein.

73. The composition of embodiment 71 or embodiment 72, wherein the mRNA is a self-replicating mRNA.

74. The composition of embodiment 73, wherein the self-replicating RNA comprises an Alphavirus replicon lacking a viral structural protein coding region.

75. The composition of embodiment 74, wherein the Alphavirus is selected from the group consisting of a Venezuelan equine encephalitis virus, a Sindbis virus, and a Semliki Forrest virus.

76. The composition of embodiment 74, wherein the Alphavirus is a Venezuelan equine encephalitis virus.

77. The composition of any one of embodiments 73-76, wherein the self-replicating mRNA is a temperature-sensitive agent (ts-agent) that is capable of expressing the fusion protein at a permissive temperature but not at a non-permissive temperature.

78. The composition of embodiment 77, wherein the permissive temperature is from 31° C. to 35° C., and the non-permissive temperature is at least 37° C.±0.5° C.

79. The composition of any one of embodiments 74-78, wherein the Alphavirus replicon comprises a nonstructural protein coding region with an insertion of 12-18 nucleotides resulting in expression of a nonstructural Protein 2 (nsP2) comprising from 4 to 6 additional amino acids between beta sheet 4 and beta sheet 6 of the nsP2.

80. The composition of any one of embodiments 71-79, wherein the first virus and/or the second virus is a coronavirus, optionally wherein the coronavirus is a betacoronavirus, optionally wherein the betacoronavirus is a human betacoronavirus.

81. The composition of embodiment 80, wherein the first and/or the second virus is a betacoronavirus independently selected from the group consisting of a severe acute respiratory syndrome coronavirus-2 (SARS-COV-2), a severe acute respiratory syndrome coronavirus-1 (SARS-COV-1), and a middle east respiratory syndrome-related coronavirus (MERS-COV).

82. The composition of embodiment 80, wherein the first virus is SARS-COV-2 and the second virus is MERS-COV.

83. The composition of any one of embodiments 80-82, wherein the surface protein, the first surface protein and/or the second surface protein each comprise a receptor-binding domain (RBD) of a coronavirus Spike protein.

84. The composition of embodiment 83, wherein the amino acid sequence of the fusion protein comprises SEQ ID NO:27, or the amino acid sequence at least 75%, 85%, 90%, 95%, 96%, 97%, 98% or 99% identical to SEQ ID NO:27.

85. The composition of any one of embodiments 71-79, wherein the first virus and/or the second virus is a member of the orthomyxoviridae family.

86. The composition of embodiment 85, wherein the first and/or the second virus is independently selected from the group consisting of an influenza A virus (IAV) and an influenza B virus (IBV).

87. The composition of embodiment 86, wherein the first virus is IAV and the second virus is IBV.

88. The composition of any one of embodiments 85-87, wherein the surface protein, the first surface protein and/or the second surface protein each comprise a portion of an influenza hemagglutinin.

89. The composition of embodiment 88, wherein the amino acid sequence of the fusion protein comprises SEQ ID NO:29, or the amino acid sequence at least 75%, 85%, 90%, 95%, 96%, 97%, 98% or 99% identical to SEQ ID NO:29.

90. The composition of any one of embodiments 71-89, wherein the composition further comprises chitosan.

91. A kit comprising:
(i) the composition of any one of embodiments 71-90; and
(ii) a device for intradermal delivery of the composition to a mammalian subject.

92. The kit of embodiment 91, wherein the device comprises a syringe and a needle.

93. The kit of embodiment 91 or embodiment 92, further comprising instructions for use of the device to administer the composition to a mammalian subject to stimulate an immune response against one or more of the first viral antigen, the second viral antigen, the third viral antigen, and the fourth viral antigen.

94. A method of stimulating an immune response in a mammalian subject, comprising administering the composition of any one of embodiments 71-90 to a mammalian subject to stimulate an immune response against one or more of the first viral antigen, the second viral antigen, the third viral antigen, and the fourth viral antigen in the mammalian subject.

95. The method of embodiment 94, wherein the composition is administered intradermally.

96. The method of embodiment 95, wherein the immune response comprises a cellular immune response reactive against one or more of the first viral antigen, the second viral antigen, the third viral antigen, and the fourth viral antigen.

97. The method of embodiment 96, wherein the immune response further comprises a humoral immune response reactive against one or more of the first viral antigen, the second viral antigen, the third viral antigen, and the fourth viral antigen.

98. The method of any one of embodiments 94-97, wherein the mammalian subject is a human subject.

99. A method for active booster immunization against at least one virus, comprising intradermally administering the composition of any one of embodiments 1-29, any one of embodiments 37-62, or any one of embodiments 71-90 to a mammalian subject in need thereof to stimulate a secondary immune response against the virus, wherein the mammalian subject had already undergone a primary immunization regimen against the virus.

100. The method of embodiment 99, wherein the primary immunization regimen comprises administration of at least one dose of a different vaccine against the virus.

101. The method of embodiment 100, wherein the different vaccine comprises a protein antigen of the at least one virus, optionally wherein the protein antigen is a recombinant protein or fragment thereof, or an inactivated virus.

102. A method for active booster immunization against at least one virus, comprising:
(i) intradermally administering the composition of any one of embodiments 1-29, any one of embodiments 37-62, or any one of embodiments 71-90 to a mammalian subject in need thereof to stimulate a primary immune response against the virus; and
(ii) administering at least one dose of a different vaccine against the virus to the mammalian subject to stimulate a secondary immune response against the virus.

103. The method of embodiment 102, wherein the different vaccine comprises a protein antigen of the at least one virus, optionally wherein the protein antigen is a recombinant protein or fragment thereof, or an inactivated virus.

104. A method for active primary immunization against at least one virus, comprising:
(i) intradermally administering the composition of any one of embodiments 1-29, any one of embodiments 37-62, or any one of embodiments 71-90 to a mammalian subject in need thereof to stimulate a primary immune response against the virus; wherein the mammalian subject had not undergone a primary immunization regimen against the virus.

105. The method of embodiment 104, further comprising:
(ii) administering at least one dose of a different vaccine against the virus to the mammalian subject to stimulate a secondary immune response against the virus.

106. The method of embodiment 105, wherein the different vaccine comprises a protein antigen of the at least one virus, optionally wherein the protein antigen is a recombinant protein or fragment thereof, or an inactivated virus.

107. The method of any one of embodiments 94-106, wherein the mammalian subject is a human subject.

108. An expression vector comprising the mRNA of any of the preceding claims in operable combination with a promoter.

109. The expression vector of embodiment 108, wherein the promoter is a T7 promoter or a SP6 promoter.

110. The expression vector of embodiment 108, wherein the vector is a plasmid.

111. The expression vector of any one of embodiments 108-110, further comprising a selectable marker.

### EXAMPLES

Abbreviations: Ab (antibody); APC (antigen presenting cell); CoV (coronavirus); c-srRNA (temperature-controllable, self-replicating RNA); CTL (cytotoxic T lymphocyte); FluA or IAV (influenza A virus); FluB or IBV (influenza B virus); IL-4 (interleukin-4); INF-γ (interferon gamma); GOI (gene of interest); HA (hemagglutinin); MERS (middle east respiratory syndrome-related); nAb (neutralizing antibody); N or NP (nucleocapsid or nucleoprotein); nsP (non-structural protein); ORF (open reading frame); PBO (placebo); RBD (receptor-binding domain); S (spike); PRNT (plaque reduction neutralization test); SARS (severe acute respiratory syndrome); SFC (spot-forming cells); SFU (spot-forming units); srRNAts (temperature-sensitive, self-replicating RNA); Th (helper T lymphocyte); and Tx (treatment). The terms c-srRNA and srRNAts are used interchangeably throughout the disclosure, with srRNA1ts2 (described in WO 2021/138447 A1) being an exemplary embodiment.

### Example 1. Cellular Immunity Induced by srRNA1ts2-G5004

This example describes the finding that SARS-COV-2 nucleoprotein alone (G5004 antigen, without a signal peptide) does not induce a potent cellular immune response when the protein is expressed from intradermally-injected, temperature-controllable, self-replicating RNA.

### Materials and Methods.

CD-1 outbred female mice.

srRNA1ts2-G5004 mRNA was produced by in vitro transcription of a temperature-controllable, self-replicating RNA vector (srRNA 1ts2 as described in PCT/US2020/067506) encoding the G5004 antigen (FIG. 2).

A pool of 102 peptides derived from a peptide scan (15mers with 11 amino acid overlaps) through Nucleoprotein (UniProt: PODTC9) of SARS-COV-2 [JPT peptide Product Code: PM-WCPV-NCAP].

ELISpot assay plates and reagents for interferon gamma (INF-γ) and interleukin-4 (IL-4) (Cellular Technology Limited, Ohio, USA).

Immunospot S6 Entry Analyzer (Cellular Technology Limited, Ohio, USA).

### Results

Recently, it has been shown that vaccination with nucleoprotein (N) alone elicits cellular immunity and spike-independent SARS-COV-2 protective immunity in mice and hamsters (Machett et al., bioRxiv. 2021.04.26.441518.2021). Vaccination involved intravenous administration of a human adenovirus serotype 5 (Ad5) vector expressing the N sequence (Ad5-N) derived from USA-WA1/2021 strain.

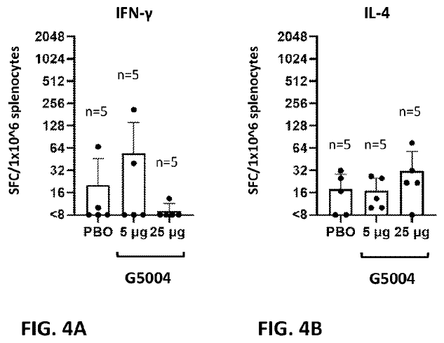

To test whether nucleoprotein (N) alone (without a signal peptide) can induce cellular immunity, ELISpot assays were performed 14 days after vaccinating CD-1 outbred mice by a single intradermal injection of either 5 μg or 25 μg of an srRNA1ts2-G5004 (FIG. 2) or a placebo (PBO: buffer only). Only weak induction of interferon-gamma (INF-γ)-secreting T cells (FIG. 4A) and IL-4-secreting T cells (FIG. 4B) was observed. Interestingly, the INF-γ response was not observed to be dose-dependent (5 μg vs. 25 μg).

It was concluded that the nucleoprotein (N) alone did not induce a potent cellular immune response when expressed from the intradermally-injected, temperature-controllable, self-replicating RNA.

### Example 2. Cellular Immunity Induced by srRNA1ts2-G5005

This example describes the finding that the addition of a CD5-signal peptide to SARS-COV-2 nucleoprotein induces a potent cellular immune response in CD-1 mice when expressed from intradermally-injected, temperature-controllable, self-replicating RNA.

### Materials and Methods

CD-1 outbred female mice.

srRNA1ts2-G5005 mRNA was produced by in vitro transcription of a temperature-controllable self-replicating RNA vector (srRNA1ts2 as disclosed in PCT/US2020/067506]) encoding the G5005 antigen (FIG. 2).

A pool of 102 peptides derived from a peptide scan (15mers with 11 amino acid overlaps) through Nucleoprotein (UniProt: PODTC9) of SARS-COV-2 [JPT peptide Product Code: PM-WCPV-NCAP].

ELISpot assay plates and reagents for interferon gamma (INF-γ) and interleukin-4 (IL-4) (Cellular Technology Limited, Ohio, USA).

Immunospot S6 Entry Analyzer (Cellular Technology Limited, Ohio, USA).

### Results

The wild-type nucleoprotein does not contain a signal peptide or a transmembrane domain, and therefore is not expected to be directed to the mammalian host cell's secretory pathway. The inventor reasoned that the lack of a signal peptide may be why the wild-type nucleoprotein (expressed from srRNA1ts2-G5004 of Example 1) did not induce a potent cellular immune response. With this in mind, the coding region of the signal peptide sequence from the human CD5 gene was added to the nucleoprotein coding region in place of the start codon (ATG) of the nucleoprotein in srRNA1ts2-G5005 (FIG. 2). The amino acid sequence of the CD5 signal peptide is MPMGSLQPLATLYLLGMLVASCLG (set forth as SEQ ID NO:8).

Cellular immunity was assessed by ELISpot assays 14 days after vaccinating CD-1 outbred mice by a single intradermal injection of either 5 μg or 25 μg of an srRNA1ts2-G5005 (FIG. 2) or a placebo (PBO: buffer only).

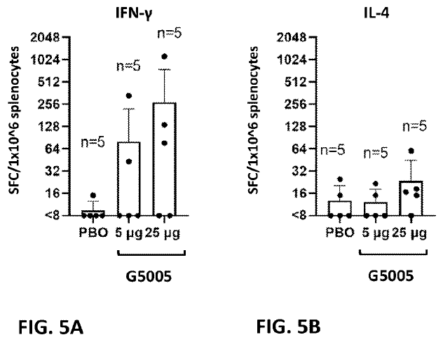

As shown in FIG. 5A, antigen-specific, INF-γ-secreting T cells were strongly induced in a dose-dependent manner (5 μg vs. 25 μg). By contrast, there was little to no induction of antigen-specific IL-4-secreting T cells (FIG. 5B). Th1 cells secrete INF-γ, while Th2 cells secrete IL-4. It is generally accepted that a Th1>Th2 immune response is a favorable feature of a vaccine.

In conclusion, addition of a signal peptide derived from human CD5 to the N-terminus of the nucleoprotein (N) resulted in induction of a strong antigen-specific cellular immune response when the protein is expressed from intradermally-injected, temperature-controllable, self-replicating RNA. The srRNA1ts2-G5005 vaccine also showed a favorable Th1-skewed (Th1>Th2) immune response.

### Example 3. Cellular Immunity Induced by srRNA1ts2-G5005

This example describes the finding that the addition of a CD5-signal peptide to the SARS-COV-2 nucleoprotein induces a potent cellular immune response in BALB/c mice when expressed from intradermally-injected, temperature-controllable, self-replicating RNA.

### Materials and Methods

BALB/c female mice.

srRNA1ts2-G5005 mRNA was produced by in vitro transcription of a temperature-controllable self-replicating RNA vector (srRNAlts2 as described in PCT/US2020/067506]) encoding the G5005 antigen (FIG. 2).

A pool of 102 peptides derived from a peptide scan (15mers with 11 amino acid overlaps) through Nucleoprotein (UniProt: PODTC9) of SARS-COV-2 [JPT peptide Product Code: PM-WCPV-NCAP].

ELISpot assay plates and reagents for interferon gamma (INF-γ) and interleukin-4 (IL-4) (Cellular Technology Limited, Ohio, USA).

Immunospot S6 Entry Analyzer (Cellular Technology Limited, Ohio, USA).

### Results

To test whether srRNA1ts2-G5005 can induce a strong cellular immune response in another mouse strain, an immunogenicity study was also conducted in BALB/c mice. Cellular immunity was assessed by ELISpot assays 30 days after vaccinating BALB/c mice by a single intradermal injection of either 5 μg or 25 μg of srRNAlts2-G5005 (FIG. 2) or a placebo (PBO: buffer only).

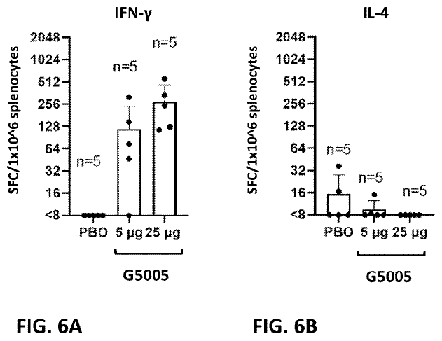

As shown in FIG. 6A, antigen-specific, INF-γ-secreting T cells was strongly induced in a dose-dependent manner (5 μg vs. 25 μg). By contrast, antigen-specific, IL-4-secreting T cells were not induced (FIG. 6B). Therefore, a favorable Th1>Th2 cellular response was also observed in BALB/c mice.

In conclusion, addition of a signal peptide derived from human CD5 to the N-terminus of the nucleoprotein (N) significantly enhanced an antigen-specific cellular immune response when the protein is expressed from intradermally-injected, temperature-controllable, self-replicating RNA. As in CD-1 mice, the srRNAlts2-G5005 vaccine showed a favorable Th1 skewed (Th1>Th2) immune response in BALB/c mice.

Example 4. Humoral immunity induced by srRNA1ts2-G5005

This example describes the finding that the SARS-COV-2 nucleoprotein when linked to the human CD5-signal peptide induces a potent humoral immune response when the protein is expressed from intradermally-injected, temperature-controllable, self-replicating RNA.

### Materials and Methods

BALB/c female mice.

srRNA1ts2-G5005 mRNA was produced by in vitro transcription of a temperature-controllable self-replicating RNA vector (srRNA1ts2 as described in PCT/US20/67506) encoding the G5005 antigen (FIG. 2).

SARS-COV-2 Nucleocapsid IgG ELISA kit (ENZO: ENZ-KIT193-0001).

### Results

To test whether srRNA1ts2-G5005 can induce a humoral immunity, nucleoprotein-specific IgG levels in serum was measured by ELISA 30 days after vaccinating BALB/c mice by a single intradermal injection of either 5 μg or 25 μg of srRNAlts2-G5005 (FIG. 2) or a placebo (PBO: buffer only). The IgG levels are represented by OD450 in the ELISA. The IgG levels were measured before (Day -1) and after (Day 30) vaccination (Day 0).

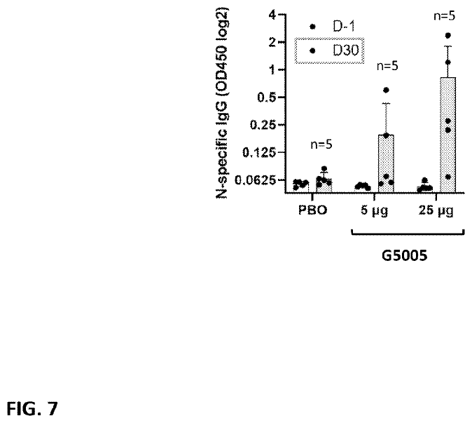

As shown in FIG. 7, nucleoprotein-specific serum IgG was strongly induced in a dose-dependent manner (5 μg vs. 25 μg).

In conclusion, addition of a signal peptide derived from human CD5 to the N-terminus of the nucleoprotein (N) induced an antigen-specific humoral immune response when the protein is expressed from intradermally-injected, temperature-controllable, self-replicating RNA.

### Example 5. Cellular Immunity Induced by srRNA1ts2-G5006

This example describes the finding that a fusion protein comprising the SARS-CoV-2 nucleoprotein and the MERS-COV nucleoprotein can induce strong cellular immunity against SARS-COV-2 and MERS-COV when the protein is expressed from intradermally-injected, temperature-controllable, self-replicating RNA.

### Materials and Methods

BALB/c female mice.

srRNA1ts2-G5006 mRNA was produced by in vitro transcription of a temperature-controllable, self-replicating, RNA vector (srRNA1ts2 as described in PCT/US2020/067506) encoding the G5006 antigen (FIG. 2C).

A pool of peptides derived from a peptide scan (15mers with 11 amino acid overlaps) through Nucleoprotein (UniProt: PODTC9) of SARS-COV-2 [JPT peptide Product Code: PM-WCPV-NCAP].

A pool of peptides derived from a peptide scan (15mers with 11 amino acid overlaps) through Nucleoprotein of MERS-COV.

ELISpot assay plates and reagents for interferon gamma (INF-γ) and interleukin-4 (IL-4) (Cellular Technology Limited, Ohio, USA).

Immunospot S6 Entry Analyzer (Cellular Technology Limited, Ohio, USA).

### Results

T-cell epitopes are present in short linear peptides, typically within the size range of 8-11 residues for MHC class I, and 10-30 residues for MHC class II. Unlike many B-cell epitopes, the 3-D conformation of T-cell epitopes is not critical to recognition by immune cell receptors. Therefore, the inventor reasoned that nucleoproteins from different betacoronavirus strains can be fused together in the absence of a lengthy linker (greater than 10 amino acids in length) for use as a vaccine antigen to elicit an immune response against different betacoronaviruses (e.g., SARS-COV-1 and their variants, SARS-COV-2 and their variants, and MERS-COV and their variants).

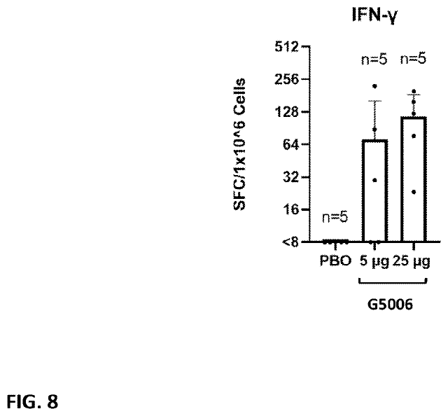

To test this concept, a fusion protein comprising a human CD5-signal peptide, a SARS-COV-2 nucleoprotein, and a MERS-COV nucleoprotein was designed (see G5006 in FIG. 2C). Mice were vaccinated with srRNA1ts2-G5006 by intradermal injection, and antigen-specific cellular immune responses were measured by ELISpot assays. As expected, the srRNA1ts2-G5006 vaccine induced a strong INF-γ-secreting T cell response against both the SARS-COV-2 nucleoprotein (FIG. 8) and the MERS-COV nucleoprotein. Additionally, the cellular immune response is expected to have a Th1>Th2 balance.

In conclusion, a fusion protein comprising nucleoproteins from different betacoronaviruses induced a strong, antigen-specific cellular immune response when the fusion protein is expressed from intradermally-injected, temperature-controllable, self-replicating RNA.

### Example 6. Cellular Immunity Induced by srRNA1ts2-G5010 (pan-Influenza vaccine)

This example describes the assessment of the immune response induced by a fusion protein comprising an Influenza A virus (FluA) nucleoprotein and an Influenza B virus (FluB) nucleoprotein when the protein is expressed from an intradermally injected temperature-controllable self-replicating RNA.

### Materials and Methods

BALB/c female mice.

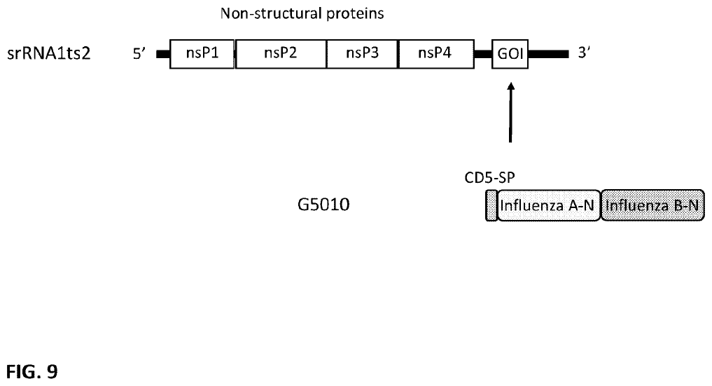

srRNA1ts2-G5010 mRNA was produced by in vitro transcription of a temperature-controllable self-replicating RNA vector (srRNA1ts2 [PCT/US20/67506]) encoding the G5010 antigen (FIG. 9). The amino acid sequence of the G5010 fusion protein is set forth as SEQ ID NO:16. The nucleic acid sequence encoding the G5010 fusion protein was codon-optimized for expression in human cells, and is set forth as SEQ ID NO:15.

A pool of 122 overlapping peptides derived from a peptide scan (15mers with 11 amino acid overlaps) through Nucleoprotein (NP) of Influenza A (H2N2) (Swiss-Prot ID P21433) [JPT peptide Product Code: PM-INFA-NPH2N2]. The amino acid sequence of the H2N2 nucleoprotein is set forth as SEQ ID NO:17.

ELISpot assay plates and reagents for interferon gamma (INF-γ) and interleukin-4 (IL-4) (Cellular Technology Limited, Ohio, USA).

Immunospot S6 Entry Analyzer (Cellular Technology Limited, Ohio, USA).

### Results

Influenza A and B can infect humans and cause seasonal epidemics or pandemics (see, “Types of Influenza Viruses” from the CDC website www.cdc.gov/flu/about/viruses/types.htm). Compared to the hemagglutinin (HA) and neuraminidase (NA) antigens that are routinely included in influenza vaccines, the nucleoprotein antigens are more conserved among different Influenza virus strains. For example, the amino acid sequences of nucleoproteins of representative Influenza A strains (H1N1, H3N2, H5N8, H7N7, H7N9, H9N2, H10N8) are very similar. Likewise, the amino acid sequences of nucleoproteins of representative Influenza B strains (Yamagata, Victoria) are very similar. In contrast, the amino acid sequences of nucleoproteins of Influenza A are significantly different from the amino acid sequences of nucleoproteins of Influenza B.

T-cell epitopes are present in short linear peptides, typically within the size range of 8-11 residues for MHC class I and 10-30 residues for MHC class II. Unlike B-cell epitopes, the conformational or 3D structure of T-cell epitopes is not critical to recognition by immune cell receptors. Therefore, one representative nucleoprotein from Influenza A is contemplated to include many T-cell epitopes shared by many Influenza A virus strains. Likewise, one representative nucleoprotein from Influenza B is contemplated to include many T-cell epitopes shared by many Influenza B virus strains. As such, the inventor reasoned that the nucleoproteins from different Influenza strains can be fused together in the absence of a lengthy linker (greater than 10 amino acids in length) for use as a vaccine antigen to elicit immune responses against different Influenza viruses (e.g., different strains of Influenza A, and different strains of Influenza B).

The amino acid sequences of nucleoproteins of representative Influenza A strains (H1N1, H3N2, H5N8, H7N7, H7N9, H9N2, and H10N8) were found to be similar to each other. The nucleoprotein of Influenza strain H5N8 was selected as it showed the fewest differences to the nucleoproteins of other strains (H1N1, H3N2, H7N7, H7N9, H9N2, and H10N8). The nucleoprotein of Influenza B strain (B/Florida/4/2006; GenBank CY033879.1) was selected as a representative Influenza B virus nucleoproteins. A fusion protein comprising a human CD5-signal peptide, one FluA nucleoprotein and one FluB nucleoprotein was designed (see, G5010 in FIG. 9), and the coding region of the fusion protein was cloned downstream of the subgenomic promoter of srRNAlts2. mRNA was subsequently produced by in vitro transcription. The amino acid sequence of the FluA nucleoprotein is set forth as SEQ ID NO:13 (Influenza Type A, H5N8 subtype [A/breeder duck/Korea/Gochang1/2014], GenBank No. KJ413835.1, ProteinID No. AHL21420.1), and the amino acid sequence of the FluB nucleoprotein is set forth as SEQ ID NO:14 (Influenza Type B [B/Florida/4/2006], GenBank No. CY033879.1, ProteinID No. ACF54251.1).

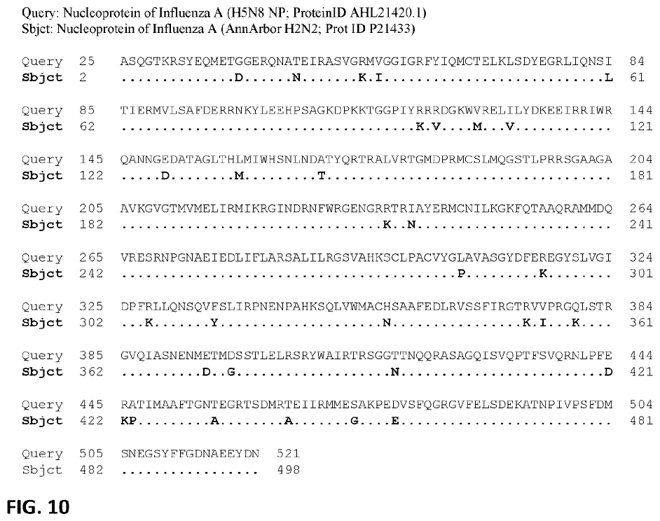

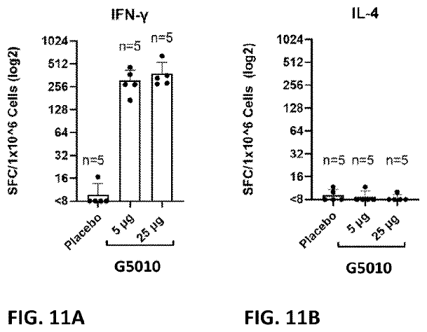

Mice were vaccinated with srRNA1ts2-G5010 by intradermal injection, and antigen-specific cellular immune responses were measured by ELISpot assays. In order to recall nucleoprotein-reactive T cell immunity, a pool of 122 overlapping peptides derived from a peptide scan of the Influenza A nucleoprotein sequence set forth as SEQ ID NO:17 were used to restimulate splenocytes harvested from mice 14 days post-vaccination. Even though, there were differences between the influenza A nucleoprotein sequence of G5010 and the influenza A nucleoprotein sequence of the peptide pool (FIG. 10), the srRNA 1ts2-G5010 vaccine induced a strong INF-γ-secreting T cell response against the FluA nucleoprotein (FIG. 11). Importantly, there was little to no induction of IL-4-secreting T cells against the FluA nucleoprotein. These results indicate that the srRNA1ts2-G5010 vaccine induces a Th1 (INF-γ)-dominant response (Th1>Th2 balance), which is a favorable feature for a vaccine directed against a viral disease.

In conclusion, a fusion protein comprising nucleoproteins from representative Influenza A and Influenza B strains induced a strong, antigen-specific cellular immune response when the fusion protein was expressed from intradermally-injected, temperature-controllable, self-replicating RNA.

### Example 7. Cellular Immunity Induced by srRNA1ts2-PanEbola (pan-Ebola Vaccine)

This example describes the finding that a fusion protein comprising fragments of nucleoproteins from four species of Ebolavirus (Zaire ebolavirus, Sudan ebolavirus, Bundibugyo ebolavirus, Taï Forest ebolavirus) can induce strong cellular immunity against Ebolaviruses when the fusion protein is used as a vaccine antigen. This example uses a temperature-controllable self-replicating RNA as an expression vector.

### Materials and Methods

BALB/c female mice.

srRNA1ts2-PanEbola mRNA was produced by in vitro transcription of a temperature-controllable self-replicating RNA vector (srRNA 1ts2 [PCT/US20/67506]) encoding a PanEbola antigen (FIG. 12).

A pool of 182 peptides derived from a peptide scan (15mers with 11 amino acid overlaps) through Nucleoprotein (Swiss-Prot ID: B8XCN6) of Ebola virus-Taï Forest Ebolavirus [JPT peptide; PepMix Tai Forest Ebolavirus (NP); JPT Product Code: PM-TEBOV-NP].

ELISpot assay plates and reagents for interferon gamma (INF-γ) and interleukin-4 (IL-4) (Cellular Technology Limited, Ohio, USA).

Immunospot S6 Entry Analyzer (Cellular Technology Limited, Ohio, USA).

### Results

Ebolaviruses cause highly lethal hemorrhagic fever. Four species of Ebolavirus are known to cause disease in humans: Ebola virus (species Zaire ebolavirus), Sudan virus (species Sudan ebolavirus), Bundibugyo virus (species Bundibugyo ebolavirus), and Taï Forest virus (species Taï Forest ebolavirus, formerly Côte d'Ivoire ebolavirus).

Currently, only one licensed vaccine (rVSV-ZEBOV) is available for Ebolavirus. This vaccine is an attenuated recombinant vesicular stomatitis virus (VSV), which expresses the main glycoprotein (GP) from the Zaire ebolavirus. Although the vaccine can induce a neutralizing antibody against Ebolavirus, the protein sequence of the GP is highly divergent among the four species of Ebolavirus, which infect humans. As such, the rVSV-ZEBOV vaccine is only effective against the Zaire ebolavirus. It is desirable to have a pan-ebolavirus vaccine, which could provide protection against all four species of ebolaviruses.

Compared to GP, the nucleoprotein (NP) sequences are more conserved among the four species of ebolavirus. However, unlike the GP, the NP is not a surface protein, and thus, the antibody induced against NP is not a neutralizing antibody. Importantly, it has been shown that mice vaccinated against Zaire ebolavirus NP can be protected from the Zaire ebolavirus challenge, which is mediated by cellular immunity, not humoral immunity (Wilson and Hart, J Virol, 75:2660-2664, 2001). It has also been shown that protection is mediated by MHC class I-restricted CD8+ killer T cells (cytotoxic T lymphocytes), not by MHC class II-restricted CD4+ helper T cells (Wilson and Hart, supra, 2001).

Using a fusion protein of NPs of all four species of ebolavirus as a vaccine antigen provides was reasoned to provide protection against all four species of ebolavirus. However, each NP is approximately 740 amino acids in length. Thus fusing four whole NPs together would result in a relatively large protein of approximately 3,000 amino acids. A smaller-sized antigen is desirable for many vaccine platforms.

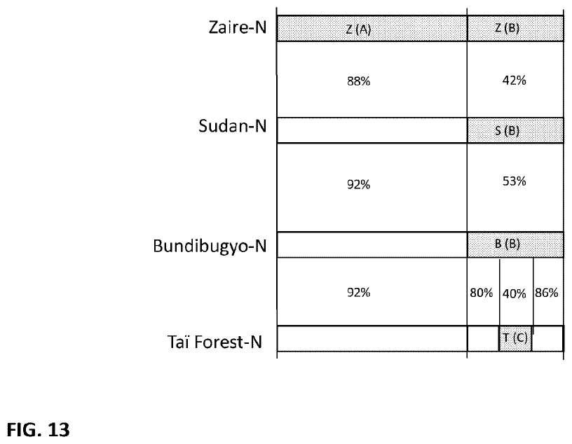

The amino acid sequences of nucleoproteins of four ebolavirus species was compared using NCBI BlastP (Zaire ebolavirus NP (GenBank ID: AF272001), Sudan ebolavirus NP (GenBank ID: AF173836), Bundibugyo ebolavirus NP (GenBank ID: FJ217161), and Taï Forest ebolavirus NP (GenBank ID: FJ217162)). The sequences of the N-terminal half of NP (termed Region A) were found to be similar to each other (88%-92% identity), whereas the sequences of the C-terminus half of NP (termed Region B) were found to be diverse (42%-54%) (Table 7-1). Therefore, Zaire (A) was chosen as a representative of Zaire (A), Sudan (A), Bundibugyo (A), and Taï Forest (A). For Region B, the Bundibugyo (B) and Taï Forest (B) were found to be similar to each other (80% and 86% identity), except for the middle part (40% identity) (termed Region C). Therefore, Zaire (B), Sudan (B), Bundibugyo (B), and Taï Forest (C) were selected for inclusion in the Pan-Ebola vaccine. Before assembling the four nucleoproteins into a single fusion protein, an additional 8 amino acid sequence was added to both sides, so that possible T-cell epitopes at the end of the nucleoprotein fragments, would not be destroyed. A schematic of the fusion protein of the Pan-Ebola antigen is shown in FIG. 12, and includes NP fragments of Zaire (A), Zaire (B), Sudan (B), Bundibugyo (B), and Taï Forest (C), as well as the human CD5 signal peptide. A diagram showing percent identities of ebolavirus NP sequences is shown in FIG. 13. The amino acid sequence of the PanEbola antigen is set forth as SEQ ID NO:22, while the nucleic acid sequence encoding the PanEbola antigen is set forth as SEQ ID NO:23.

TABLE 7-1
Percent Identity Between Domains of Ebolavirus NP Sequences

| Virus | Zaire (A) | Zaire (B) |
|---|---|---|
| Sudan | 88% | 42% |
| Bundibugyo | 92% | 53% |
| Tai Forest | 92% | |

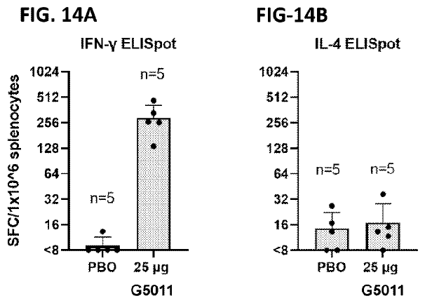

The srRNA1ts2-PanEbola vaccine was produced by cloning the PanEbola fusion protein downstream of the subgenomic promoter of a srRNAlts2. mRNA was produced by in vitro transcription, and used to vaccinate BALB/c mice intradermally. Antigen-specific cellular immune responses were measured by ELISpot assays. In order to recall nucleoprotein-reactive T cell immunity, a pool of 182 peptides derived from a peptide scan of the nucleoprotein ((Swiss-Prot ID: B8XCN6) of Ebola virus - Taï Forest Ebolavirus)) were used to restimulate splenocytes harvested from mice 14 days post-vaccination. The srRNA1ts2-PanEbola vaccine induced a strong INF-γ-secreting T cell response against the Taï Forest nucleoprotein (FIG. 14A). This is striking in that only a small part (169 aa) of the Taï Forest nucleoprotein was included in the mRNA vaccine, whereas the peptide pool used for restimulation covered the entire Taï Forest nucleoprotein sequence. Importantly, there was little to no induction of IL-4-secreting T cells against the Taï Forest nucleoprotein (FIG. 14B). These results indicate that the srRNAlts2-PanEbola vaccine induces a Th1 (INF-γ)-dominant response (Th1>Th2 balance), which is a favorable feature for a vaccine directed against a viral disease.

In conclusion, a fused protein comprising nucleoproteins fragments from four species of Ebolavirus induced a strong, antigen-specific cellular immune response when the fusion protein was expressed from intradermally-injected, temperature-controllable, self-replicating RNA. The example demonstrates that the size of a fusion protein to be used as a Pan-Ebola vaccine can be reduced by removing the more well-conserved portions of one or more of the nucleoproteins comprising the vaccine. The PanEbola antigen is also suitable for use in other vaccine platforms (e.g., adenovirus, adeno-associated virus, recombinant protein, etc.).

### Example 8. Cellular Immunity Induced by srRNA1ts2-G50030 (Omicron Vaccine)

This example describes the finding that intradermal delivery of c-srRNA encoding the RBD of SARS-COV-2 (omicron strain B.1.1.529) can induce strong cellular immunity in mice.

### Materials and Methods

C57BL/6 female mice.

An srRNA1ts2-G50030 (mRNA), which was produced by in vitro transcription of a temperature-controllable self-replicating RNA vector (srRNA1ts2 [WO 2021/138447 A1]) encoding the G50030 antigen (FIG. 15).

A pool of peptides derived from a peptide scan (15mers with 11 amino acid overlaps) through RBD of SARS-COV-2 Omicron variant (S-RBD B.1.1.529) [JPT Peptides: PM-SARS2-RBDMUT08-1]

ELISpot assay plates and reagents for interferon gamma (INF-γ) and interleukin-4 (IL-4) (Cellular Technology Limited, Ohio, USA).

Immunospot S6 Entry Analyzer (Cellular Technology Limited, Ohio, USA).

### Results

In this example, c-srRNA encoding the RBD of SARS-COV-2 omicron variant (G50030) was generated (FIG. 15). The RNA was intradermally administered to C57BL/6 mice and 14 days later the splenocytes were collected to examine the cellular immunity against SARS-CoV-2 RBD (omicron variant). Induction of INF-γ-secreting T cells was specifically observed in c-srRNA-G5003o recipients (FIG. 16A), whereas induction of IL-4-secreting T cells was not observed in c-srRNA-G5003o recipients (FIG. 16B).

### Conclusion

We have demonstrated by using an omicron variant-specific RBD as an antigen, that an omicron variant-specific cellular immune response can be induced when the protein is expressed from the intradermally injected temperature-controllable self-replicating RNA. A favorable Th1 (INF-γ) >Th2 (IL-4) response was also observed.

### Example 9. Efficacy of c-srRNA Prime, Protein Boost Immunization Regimen

This example describes the finding that administration of a c-srRNA vaccine encoding a protein antigen of an original virus is able to prime a humoral immune response to a protein antigen of a variant virus.

### Materials and Methods

BALB/c female mice.

An srRNA1ts2-G5003 (mRNA), which was produced by in vitro transcription of a temperature-controllable self-replicating RNA vector (srRNA1ts2 [WO 2021/138447 A1]) encoding the G5003 antigen (FIG. 15).

Recombinant SARS-COV-2 B.1.617.2 Spike GCN4-IZ Protein (R&D Systems, Cat. #10878-CV)

Adda Vax™ squalene-based oil-in-water adjuvant was obtained from InvivoGen.

A pool of peptides derived from a peptide scan (15mers with 11 amino acid overlaps) through RBD of SARS-COV-2 (an original Wuhan strain) [JPT Peptides: PepMix SARS-COV-2 (S-RBD) PM-WCPV-S-RBD-2]

ELISpot assay plates and reagents for interferon gamma (INF-γ) and interleukin-4 (IL-4) (Cellular Technology Limited, Ohio, USA).

Immunospot S6 Entry Analyzer (Cellular Technology Limited, Ohio, USA).

Vero76 cells for a plaque reduction neutralization assay (PRNT).

SARS-COV-2 Delta Variant live virus for the PRNT assay

For the PRNT assay, Vero76 cells were first treated with serially diluted mouse serum, followed by the infection with a live virus of SARS-COV-2 (Delta variant strain). In this assay, the infected cells die and form a plaque after fixation and staining with crystal violet. If the serum contains the neutralizing antibodies, the viral infection is inhibited, resulting in the reduction of the number of plaques. The results are shown as the dilution titer of serum that show 50% reduction of number of plaques (PRNT50).

### Results

A composition comprising the c-srRNA encoding G5003 antigen (RBD of SARS-CoV-2 original Wuhan strain) was administered intradermally into skin of BALB/c mice as naked mRNA (FIG. 17A). That is, the srRNA1ts2-G5003 composition did not contain any nanoparticles or transfection reagents. Subsequently, a composition comprising the Spike protein of SARS-COV-2 (Delta variant B.1.617.2) mixed with adjuvant was administered intradermally (FIG. 17A).

Cellular immunity against the SARS-COV-2 RBD protein was detected in mouse splenocytes 14 days after a single intradermal injection of the c-srRNA-G5003 composition (FIG. 17B). Subsequent exposure of immunized mice to a spike protein of a different SARS-CoV-2 strain (Delta variant B.1.617.2) induced neutralization antibodies (detected by the PRNT assay) against the Delta variant of SARS-COV-2 (FIG. 17C) as early as day 7 post-protein antigen exposure. In contrast, mice that did not receive c-srRNA-G5003 encoding the RBD of the SARS-COV-2 (an original Wuhan strain) did not mount a neutralizing antibody response to the Delta variant of SARS-COV-2. The early induction of neutralizing antibodies is characteristic of a secondary immune response, indicating that the c-srRNA primed the humoral immune response prior to exposure to the adjuvanted RBD protein.

### Conclusion

The results indicate that the c-srRNA immunogen can induce a potent immune response that is broadly reactive against both the antigen encoded by the c-srRNA and a distinct variant antigen. Thus, the c-srRNA SARS-COV-2 RBD immunogen is suitable for use in immunization regimens directed against a broad spectrum of SARS-COV-2 strains.

### Example 10. Efficacy of Protein Prime, c-srRNA Boost Immunization Regimen

This example describes the finding that a c-srRNA vaccine can enhance the antibody titer, when used as a booster vaccine for other vaccines.

### Materials and Methods

C57BL/6 female mice.

RBD protein (Sino Biological SARS-COV-2 [2019-nCOV] Spike RBD-His

Recombinant Protein, Cat. #40592-V08B)

Adda VaxTM squalene-based oil-in-water adjuvant was obtained from InvivoGen.

An srRNA1ts2-G5003 (mRNA), which was produced by in vitro transcription of a temperature-controllable self-replicating RNA vector (srRNA1ts2 [WO 2021/138447 A1]) encoding the G5003 antigen (FIG. 15).

An srRNA1ts2-G50030 (mRNA), which was produced by in vitro transcription of a temperature-controllable self-replicating RNA vector (srRNA1ts2 [WO 2021/138447 A1]) encoding the G50030 antigen (FIG. 15).

A pool of peptides derived from a peptide scan (15mers with 11 amino acid overlaps) through RBD of SARS-COV-2 (an original Wuhan strain) [JPT Peptides: PepMix SARS-COV-2 (S-RBD) PM-WCPV-S-RBD-2]

ELISpot assay plates and reagents for interferon gamma (INF-γ) and interleukin-4 (IL-4) (Cellular Technology Limited, Ohio, USA).

Immunospot S6 Entry Analyzer (Cellular Technology Limited, Ohio, USA).

ELISA assay plates (ENZO SARS-COV-2 IgG ELISA Kit [Cat. # ENZ-KIT170-0001, the plate was coated with SARS-COV-2 (Wuhan strain) S1 antigen RBD protein]).

### Results

To test a possibility whether c-srRNA vaccine can be used as a booster vaccine, mice were first vaccinated with adjuvanted protein (in this case, RBD of SARS-COV-2 [an original Wuhan strain]). Fourteen days later (Day 14), the mice were further treated with intradermal injection of a placebo (PBO: buffer only), c-srRNA encoding G5003 antigen, c-srRNA encoding G50030 antigen, or the adjuvanted RBD protein (FIG. 18A).

On Day 28, cellular immunity was assessed by the ELISpot assay. As expected, the RBD (1st) +PBO (2nd) group could not induce the cellular immunity, whereas the RBD (1st) +RBD (2nd) group induced the cellular immunity (FIG. 18B, C). Interestingly, the RBD (1st) +c-srRNA-G5003 and c-srRNA-G50030 groups also induced the cellular immunity (FIG. 18B, C). This was expected, as c-srRNA vaccine alone can induce the cellular immunity.

On Day 28, the levels of serum antibodies against the RBD of the SARS-COV-2 virus (an original Wuhan strain) was assessed by an ELISA assay (FIG. 19). The first vaccination with the adjuvanted RBD protein alone could induce the antibody weakly. On the other hand, c-srRNA vaccines was able to induce the antibodies at the level, similar to that by the second vaccination with the adjuvanted protein.

### Conclusion

The results indicate that the c-srRNA vaccine can work as a booster vaccine for both cellular immunity and humoral immunity.

Example 11. Potent Cellular Immune Response Induced by srRNA1ts2-G5006

This example describes the finding that a fusion protein comprising the SARS-CoV-2 nucleoprotein and the MERS-COV nucleoprotein can induce strong cellular immunity against SARS-COV-2 and MERS-COV when the protein is expressed from intradermally-injected, temperature-controllable, self-replicating RNA. The vaccinated mice can eliminate the implanted tumor cells expressing a fusion protein comprising the SARS-COV-2 nucleoprotein and the MERS-COV nucleoprotein.

### Materials and Methods

BALB/c female mice.

srRNA1ts2-G5006 mRNA was produced by in vitro transcription of a temperature-controllable, self-replicating, RNA vector (srRNAlts2 as described in WO 2021/138447 A1) encoding the G5006 antigen (FIG. 2).

A pool of peptides derived from a peptide scan (15mers with 11 amino acid overlaps) through Nucleoprotein (UniProt: PODTC9) of SARS-COV-2 [JPT peptide Product Code: PM-WCPV-NCAP].

A pool of peptides derived from a peptide scan (15mers with 11 amino acid overlaps) through Nucleoprotein of MERS-COV (YP_009047211.1). The peptides were custom-made by JPT Peptides.

ELISpot assay plates and reagents for interferon gamma (INF-γ) and interleukin-4 (IL-4) (Cellular Technology Limited, Ohio, USA).

Immunospot S6 Entry Analyzer (Cellular Technology Limited, Ohio, USA).

4T1 breast cancer cell line, derived from BALB/c mouse and known as a model for a triple-negative stage IV human breast cancer, was purchased from ATCC (catalog # CRL-2539).

A plasmid DNA, encoding a fusion protein of nucleoproteins of SARS-COV-2 and MERS-COV (non-secreted form of G5006, i.e., without CD5 signal peptides) under the CMV promoter, and hygromycin-resistant gene under the promoter of SV40 early promoter, was transfected to 4T1 cells. Cells, expressing the fusion protein of nucleoproteins of SARS-COV-2 and MERS-COV (called 4T1-SMN), were isolated by culturing the cells in the presence of 200 μg/mL of hygromycin B.

### Results

To model cells infected with a virus, we used a 4T1 breast cancer cell line, derived from BALB/c mouse and known as a model for a triple-negative stage IV human breast cancer. When injected into BALB/c mouse, the 4T1 cells grow rapidly and form tumors. This syngenic mouse model was used to mimic the rapid increase of infected cells. To this end, we first made a plasmid vector encoding a fusion protein of nucleoproteins of SARS-COV-2 and MERS-COV (named SMN protein), under the CMV promoter, so that the protein is constitutively expressed. This fusion protein is the same as G5006, but the CD5 signal peptides were removed from the N-terminus of the protein. Naturally, nucleoprotein does not have the signal peptides and stays within the cytoplasm of the cells. The 4T1 cells expressing the SMN protein (named 4T1-SMN) was established after the hygromycin selection, as the plasmid vector also carried the hygromycin-resistant gene.

BALB/c mice were vaccinated with c-srRNA-G5006, and the induction of cellular immunity was demonstrated by the presence of T-cells that responded to both SARS-CoV-2 nucleoprotein (FIG. 20A) and MERS-COV nucleoprotein (FIG. 20B).

4T1-SMN cells were injected into the BALB/c mice vaccinated with c-srRNA-G5006 on day 24 (24 days post-vaccination) (FIG. 21). As expected, 4T1-SMN cells grew rapidly mice that received a placebo (no vaccination group) 4T1-SMN cells. On the other hand, the growth of 4T1-SMN tumors were suppressed in the c-srRNA-G5006 vaccinated mice. Two mice received 25 μg of the c-srRNA-G5006 vaccine, though the tumor grew initially, became tumor-free and survived. Furthermore, even after the second round of injection of 4T1-SMN tumors on day 143 after the vaccination, no tumors grew, and the mice were tumor-free and continued to live (FIG. 21).

### Conclusion

c-srRNA vaccine can induce strong cellular immunity, which can kill and eliminating cells that express the antigen. This result indicates that c-srRNA functions as a vaccine by eliminating the infected cells.

### Example 12. Cellular Immunity Induced by srRNA1ts2-PanCoronavirus Vaccine

This example describes the finding that a fusion protein comprising the CD5 signal peptides, Spike-RBD of SARS-COV-2, nucleoprotein of SARS-COV-2, nucleoprotein of MERS-COV, and Spike-RBD of MERS-COV can induce strong cellular immunity against all of these antigens, when the protein is expressed from intradermally-injected, temperature-controllable, self-replicating RNA.

### Materials and Methods

C57BL/6 female mice.

srRNA1ts2-G5006 mRNA was produced by in vitro transcription of a temperature-controllable, self-replicating, RNA vector (srRNAlts2 as described in WO 2021/138447 A1) encoding the G5006 antigen (FIG. 2).

srRNA1ts2-G5006d mRNA was produced by in vitro transcription of a temperature-controllable, self-replicating, RNA vector (srRNAlts2 as described in WO 2021/138447 A1) encoding the G5006d antigen (FIG. 22).

A pool of peptides derived from a peptide scan (15mers with 11 amino acid overlaps) through RBD of SARS-COV-2 (an original Wuhan strain) [JPT Peptides: PepMix SARS-COV-2 (S-RBD) PM-WCPV-S-RBD-2]

A pool of peptides derived from a peptide scan (15mers with 11 amino acid overlaps) through Nucleoprotein (UniProt: PODTC9) of SARS-COV-2 [JPT peptide Product Code: PM-WCPV-NCAP].

A pool of peptides derived from a peptide scan (15mers with 11 amino acid overlaps) through Nucleoprotein of MERS-COV (YP_009047211.1). The peptides were custom-made by JPT Peptides.

A pool of 336 (168+168) peptides derived from a peptide scan (15mers with 11 amino acid overlaps) through Spike glycoprotein (Swiss-Prot ID: K9N5Q8) of MERS-COV (Middle East respiratory syndrome-related coronavirus) [JPT peptides Product Code: PM-MERS-COV-S-1].

ELISpot assay plates and reagents for interferon gamma (INF-γ) and interleukin-4 (IL-4) (Cellular Technology Limited, Ohio, USA).

Immunospot S6 Entry Analyzer (Cellular Technology Limited, Ohio, USA).

### Results

We here designed a new booster vaccine, which is a c-srRNA vaccine (called c-srRNA-G5006d) encoding a fusion protein comprising the CD5 signal peptides, Spike-RBD of SARS-COV-2, nucleoprotein of SARS-COV-2, nucleoprotein of MERS-COV, and Spike-RBD of MERS-COV (FIG. 22).

Mice were vaccinated with the intradermal injection of a placebo (PBO: buffer only), c-srRNA encoding G5006 antigen, and c-srRNA encoding G5006d antigen. On day 14 post-vaccination, cellular immunity was assessed by ELISpot assays.

As shown in FIG. 23A, c-srRNA-G5006d can stimulate cellular immunity against all the proteins encoded on this vaccine: Spike-RBD of SARS-COV-2, Nucleoprotein of SARS-CoV-2, Nucleoprotein of MERS-COV, and Spike-RBD of MERS-COV.

### Conclusion

The results indicate that the c-srRNA vaccine can work as a booster vaccine for both cellular immunity and humoral immunity.

### Example 13. srRNA1ts2-PanInfluenza Virus Vaccine

This example describes the design of pan-influenza booster vaccine based on the unique feature of c-srRNA vaccine platform. An antigen (G5012) encoded on c-srRNA is a fusion protein of CD5 signal peptide (residues 1-24), a part of the hemagglutinin (HA) of the Influenza A, nucleoprotein of Influenza A, nucleoprotein of Influenza B, and a part of the hemagglutinin (HA) of the Influenza B.

### Materials and Methods

C57BL/6 female mice.

c-srRNA-G5012 mRNA was produced by in vitro transcription of a temperature-controllable, self-replicating, RNA vector (srRNAlts2 as described in WO 2021/138447 A1) encoding the G5012 antigen (FIG. 24).

Pools of peptides derived from a peptide scan (15mers with 11 amino acid overlaps) through a part of the hemagglutinin (HA) of the Influenza A, nucleoprotein of Influenza A, nucleoprotein of Influenza B, and a part of the hemagglutinin (HA) of the Influenza B.

ELISpot assay plates and reagents for interferon gamma (INF-γ) and interleukin-4 (IL-4) (Cellular Technology Limited, Ohio, USA).

Immunospot S6 Entry Analyzer (Cellular Technology Limited, Ohio, USA).

### Results

Mice were vaccinated with the intradermal injection of a placebo (PBO: buffer

only), and c-srRNA encoding G5012 antigen. On day 14 post-vaccination, cellular immunity was assessed by ELISpot assays.

c-srRNA-G5012 stimulated cellular immunity against all the antigen encoded on this vaccine: the hemagglutinin (HA) of the Influenza A, the nucleoprotein of Influenza A, the nucleoprotein of Influenza B, and the hemagglutinin (HA) of the Influenza B.

### Conclusion

The results indicate that the c-srRNA vaccine can work as a booster vaccine for both cellular immunity and humoral immunity.

### Example 14. Chitosan-Enhanced Luciferase Expression from srRNA1ts2-LUC2

This example describes the finding that chitosan oligomers are able to enhance in vivo expression of a gene of interest (GOI) encoded by a c-srRNA construct.

### Materials and Methods

C57BL/6 female mice.

An srRNA1ts2-LUC2 (mRNA), which was produced by in vitro transcription of a temperature-controllable self-replicating RNA vector (srRNAlts2 as described in WO 2021/138447 A1) encoding the luciferase gene.

Chitosan Oligomer (molecular weight ≤5 kDa, ≥75.0% deacetylated: Heppe Medical Chitosan GmbH: Product No. 44009)

Chitosan oligosaccharide lactate (molecular weight ˜5 kDa, >90% deacetylated: Sigma-Aldrich: Product No. 523682)

Bioluminescent Imaging system, AMI HTX (Spectral Instruments Imaging, Tucson, AZ)

### Results

To test whether chitosan oligomers can enhance the expression of GOI encoded on c-srRNA in vivo, 5 μg of c-srRNA (also known as srRNA 1ts2) encoding a luciferase gene as GOI was mixed with chitosans and administered intradermally to each C57BL/6 mouse (FIG. 25). c-srRNAs were formulated as naked RNAs, without lipid nanoparticles or any other transfection reagents, in lactated Ringer's solution. Luciferase activity was visualized and quantitated by using a bioluminescent Imaging system, AMI HTX (Spectral Instruments Imaging, Tucson, AZ).

Five mice each were tested in the following groups: 1, a control - c-srRNA only; 2, c-srRNA mixed with chitosan oligosaccharide (0.001 μg/mL); 3, c-srRNA mixed with chitosan oligosaccharide (0.01 μg/mL); 4, c-srRNA mixed with chitosan oligosaccharide (0.5 μg/mL); 5, c-srRNA mixed with chitosan oligosaccharide lactate (0.1 μg/mL).

As shown in FIG. 27, compared to the control condition (i.e., c-srRNA only: no chitosan), all the conditions with chitosan oligomers at the concentration of 0.001 μg/mL, 0.01 μg/mL, and 0.5 μg/mL as well as the condition with chitosan oligosaccharide lactate at the concentration of 0.1 μg/mL showed ˜10-fold higher levels of luciferase activity.

### Conclusion

Low-molecular-weight chitosans such as chitosan oligomers and chitosan oligosaccharide lactate can enhance the expression of GOI encoded on c-srRNA, when mixed with c-srRNA before injecting c-arRNA into mouse skin intradermally. Chitosan oligomers provide about a 10-fold enhancement of gene expression even at a very low concentration (0.001 μg/mL or about 0.2 nM). This surprising discovery provides an effective means to enhance the in vivo therapeutic expression of GOI encoded on c-srRNA.

SEQUENCES
>G5004 (SARS-COV-2_N, mRNA)
SEQ ID NO: 1
ATGTCTGATAATGGACCCCAAAATCAGCGAAATGCACCCCGCATTACG
TTTGGTGGACCCTCAGATTCAACTGGCAGTAACCAGAATGGAGAACGC
AGTGGGGCGCGATCAAAACAACGTCGGCCCCAAGGTTTACCCAATAAT
ACTGCGTCTTGGTTCACCGCTCTCACTCAACATGGCAAGGAAGACCTT
AAATTCCCTCGAGGACAAGGCGTTCCAATTAACACCAATAGCAGTCCA
GATGACCAAATTGGCTACTACCGAAGAGCTACCAGACGAATTCGTGGT
GGTGACGGTAAAATGAAAGATCTCAGTCCAAGATGGTATTTCTACTAC
CTAGGAACTGGGCCAGAAGCTGGACTTCCCTATGGTGCTAACAAAGAC
GGCATCATATGGGTTGCAACTGAGGGAGCCTTGAATACACCAAAAGAT
CACATTGGCACCCGCAATCCTGCTAACAATGCTGCAATCGTGCTACAA
CTTCCTCAAGGAACAACATTGCCAAAAGGCTTCTACGCAGAAGGGAGC
AGAGGCGGCAGTCAAGCCTCTTCTCGTTCCTCATCACGTAGTCGCAAC
AGTTCAAGAAATTCAACTCCAGGCAGCAGTAGGGGAACTTCTCCTGCT
AGAATGGCTGGCAATGGCGGTGATGCTGCTCTTGCTTTGCTGCTGCTT
GACAGATTGAACCAGCTTGAGAGCAAAATGTCTGGTAAAGGCCAACAA
CAACAAGGCCAAACTGTCACTAAGAAATCTGCTGCTGAGGCTTCTAAG
AAGCCTCGGCAAAAACGTACTGCCACTAAAGCATACAATGTAACACAA
GCTTTCGGCAGACGTGGTCCAGAACAAACCCAAGGAAATTTTGGGGAC
CAGGAACTAATCAGACAAGGAACTGATTACAAACATTGGCCGCAAATT
GCACAATTTGCCCCCAGCGCTTCAGCGTTCTTCGGAATGTCGCGCATT
GGCATGGAAGTCACACCTTCGGGAACGTGGTTGACCTACACAGGTGCC
ATCAAATTGGATGACAAAGATCCAAATTTCAAAGATCAAGTCATTTTG
CTGAATAAGCATATTGACGCATACAAAACATTCCCACCAACAGAGCCT
AAAAAGGACAAAAAGAAGAAGGCTGATGAAACTCAAGCCTTACCGCAG
AGACAGAAGAAACAGCAAACTGTGACTCTTCTTCCTGCTGCAGATTTG
GATGATTTCTCCAAACAATTGCAACAATCCATGAGCAGTGCTGACTCA
ACTCAGGCCTAA
>G5005 (CD5-SP_SARS-COV-2_N, synthetic mRNA)
SEQ ID NO: 2
ATGCCCATGGGGTCTCTGCAACCGCTGGCCACCTTGTACCTGCTGGGG
ATGCTGGTCGCTTCCTGCCTCGGATCTGATAATGGACCCCAAAATCAG
CGAAATGCACCCCGCATTACGTTTGGTGGACCCTCAGATTCAACTGGC
AGTAACCAGAATGGAGAACGCAGTGGGGCGCGATCAAAACAACGTCGG
CCCCAAGGTTTACCCAATAATACTGCGTCTTGGTTCACCGCTCTCACT
CAACATGGCAAGGAAGACCTTAAATTCCCTCGAGGACAAGGCGTTCCA
ATTAACACCAATAGCAGTCCAGATGACCAAATTGGCTACTACCGAAGA
GCTACCAGACGAATTCGTGGTGGTGACGGTAAAATGAAAGATCTCAGT
CCAAGATGGTATTTCTACTACCTAGGAACTGGGCCAGAAGCTGGACTT
CCCTATGGTGCTAACAAAGACGGCATCATATGGGTTGCAACTGAGGGA
GCCTTGAATACACCAAAAGATCACATTGGCACCCGCAATCCTGCTAAC
AATGCTGCAATCGTGCTACAACTTCCTCAAGGAACAACATTGCCAAAA
GGCTTCTACGCAGAAGGGAGCAGAGGCGGCAGTCAAGCCTCTTCTCGT
TCCTCATCACGTAGTCGCAACAGTTCAAGAAATTCAACTCCAGGCAGC
AGTAGGGGAACTTCTCCTGCTAGAATGGCTGGCAATGGCGGTGATGCT
GCTCTTGCTTTGCTGCTGCTTGACAGATTGAACCAGCTTGAGAGCAAA
ATGTCTGGTAAAGGCCAACAACAACAAGGCCAAACTGTCACTAAGAAA
TCTGCTGCTGAGGCTTCTAAGAAGCCTCGGCAAAAACGTACTGCCACT
AAAGCATACAATGTAACACAAGCTTTCGGCAGACGTGGTCCAGAACAA
ACCCAAGGAAATTTTGGGGACCAGGAACTAATCAGACAAGGAACTGAT
TACAAACATTGGCCGCAAATTGCACAATTTGCCCCCAGCGCTTCAGCG
TTCTTCGGAATGTCGCGCATTGGCATGGAAGTCACACCTTCGGGAACG
TGGTTGACCTACACAGGTGCCATCAAATTGGATGACAAAGATCCAAAT
TTCAAAGATCAAGTCATTTTGCTGAATAAGCATATTGACGCATACAAA
ACATTCCCACCAACAGAGCCTAAAAAGGACAAAAAGAAGAAGGCTGAT
GAAACTCAAGCCTTACCGCAGAGACAGAAGAAACAGCAAACTGTGACT
CTTCTTCCTGCTGCAGATTTGGATGATTTCTCCAAACAATTGCAACAA
TCCATGAGCAGTGCTGACTCAACTCAGGCCTAA
>G5006A (CD5-SP_SARS2-N_MERS-N, synthetic mRNA)
SEQ ID NO: 3
ATGCCCATGGGGTCTCTGCAACCGCTGGCCACCTTGTACCTGCTGGGG
ATGCTGGTCGCTTCCTGCCTCGGATCTGATAATGGACCCCAAAATCAG
CGAAATGCACCCCGCATTACGTTTGGTGGACCCTCAGATTCAACTGGC
AGTAACCAGAATGGAGAACGCAGTGGGGCGCGATCAAAACAACGTCGG
CCCCAAGGTTTACCCAATAATACTGCGTCTTGGTTCACCGCTCTCACT
CAACATGGCAAGGAAGACCTTAAATTCCCTCGAGGACAAGGCGTTCCA
ATTAACACCAATAGCAGTCCAGATGACCAAATTGGCTACTACCGAAGA
GCTACCAGACGAATTCGTGGTGGTGACGGTAAAATGAAAGATCTCAGT
CCAAGATGGTATTTCTACTACCTAGGAACTGGGCCAGAAGCTGGACTT
CCCTATGGTGCTAACAAAGACGGCATCATATGGGTTGCAACTGAGGGA
GCCTTGAATACACCAAAAGATCACATTGGCACCCGCAATCCTGCTAAC
AATGCTGCAATCGTGCTACAACTTCCTCAAGGAACAACATTGCCAAAA
GGCTTCTACGCAGAAGGGAGCAGAGGCGGCAGTCAAGCCTCTTCTCGT
TCCTCATCACGTAGTCGCAACAGTTCAAGAAATTCAACTCCAGGCAGC
AGTAGGGGAACTTCTCCTGCTAGAATGGCTGGCAATGGCGGTGATGCT
GCTCTTGCTTTGCTGCTGCTTGACAGATTGAACCAGCTTGAGAGCAAA
ATGTCTGGTAAAGGCCAACAACAACAAGGCCAAACTGTCACTAAGAAA
TCTGCTGCTGAGGCTTCTAAGAAGCCTCGGCAAAAACGTACTGCCACT
AAAGCATACAATGTAACACAAGCTTTCGGCAGACGTGGTCCAGAACAA
ACCCAAGGAAATTTTGGGGACCAGGAACTAATCAGACAAGGAACTGAT
TACAAACATTGGCCGCAAATTGCACAATTTGCCCCCAGCGCTTCAGCG
TTCTTCGGAATGTCGCGCATTGGCATGGAAGTCACACCTTCGGGAACG
TGGTTGACCTACACAGGTGCCATCAAATTGGATGACAAAGATCCAAAT
TTCAAAGATCAAGTCATTTTGCTGAATAAGCATATTGACGCATACAAA
ACATTCCCACCAACAGAGCCTAAAAAGGACAAAAAGAAGAAGGCTGAT
GAAACTCAAGCCTTACCGCAGAGACAGAAGAAACAGCAAACTGTGACT
CTTCTTCCTGCTGCAGATTTGGATGATTTCTCCAAACAATTGCAACAA
TCCATGAGCAGTGCTGACTCAACTCAGGCCATGGCATCCCCTGCTGCA
CCTCGTGCTGTTTCCTTTGCCGATAACAATGATATAACAAATACAAAC
CTATCTCGAGGTAGAGGACGTAATCCAAAACCACGAGCTGCACCAAAT
AACACTGTCTCTTGGTACACTGGGCTTACCCAACACGGGAAAGTCCCT
CTTACCTTTCCACCTGGGCAGGGTGTACCTCTTAATGCCAATTCTACC
CCTGCGCAAAATGCTGGGTATTGGCGGAGACAGGACAGAAAAATTAAT
ACCGGGAATGGAATTAAGCAACTGGCTCCCAGGTGGTACTICTACTAC
ACTGGAACTGGACCCGAAGCAGCACTCCCATTCCGGGCTGTTAAGGAT
GGCATCGTTTGGGTCCATGAAGATGGCGCCACTGATGCTCCTTCAACT
TTTGGGACGCGGAACCCTAACAATGATTCAGCTATTGTTACACAATTC
GCGCCCGGTACTAAGCTTCCTAAAAACTTCCACATTGAGGGGACTGGA
GGCAATAGTCAATCATCTTCAAGAGCCTCTAGCTTAAGCAGAAACTCT
TCCAGATCTAGTTCACAAGGTTCAAGATCAGGAAACTCTACCCGCGGC
ACTTCTCCAGGTCCATCTGGAATCGGAGCAGTAGGAGGTGATCTACTT
TACCTTGATCTTCTGAACAGACTACAAGCCCTTGAGTCTGGCAAAGTA
AAGCAATCGCAGCCAAAAGTAATCACTAAGAAAGATGCTGCTGCTGCT
AAAAATAAGATGCGCCACAAGCGCACTTCCACCAAAAGTTTCAACATG
GTGCAAGCTTTTGGTCTTCGCGGACCAGGAGACCTCCAGGGAAACTTT
GGTGATCTTCAATTGAATAAACTCGGCACTGAGGACCCACGTTGGCCC
CAAATTGCTGAGCTTGCTCCTACAGCCAGTGCTTTTATGGGTATGTCG
CAATTTAAACTTACCCATCAGAACAATGATGATCATGGCAACCCTGTG
TACTTCCTTCGGTACAGTGGAGCCATTAAACTTGACCCAAAGAATCCC
AACTACAATAAGTGGTTGGAGCTTCTTGAGCAAAATATTGATGCCTAC
AAAACCTTCCCTAAGAAGGAAAAGAAACAAAAGGCACCAAAAGAAGAA
TCAACAGACCAAATGTCTGAACCTCCAAAGGAGCAGCGTGTGCAAGGT
AGCATCACTCAGCGCACTCGCACCCGTCCAAGTGTTCAGCCTGGTCCA
ATGATTGATGTTAACACTGATTAG
>G5006B (CD5-SP_SARS2-N_MERS-N: CODON-OPTIMIZED,
synthetic mRNA)
SEQ ID NO : 4
ATGCCTATGGGCTCTCTGCAGCCCCTGGCCACCCTGTACCTGCTGGGC
ATGCTGGTGGCCAGCTGCCTGGGAAGCGACAACGGCCCCCAGAACCAG
AGAAACGCCCCTAGAATCACATTTGGCGGCCCTAGTGATAGCACCGGA
TCTAATCAAAACGGCGAGAGAAGCGGCGCTCGGTCTAAACAGAGACGG
CCACAGGGACTGCCTAACAACACCGCCAGCTGGTTCACCGCCCTGACC
CAGCACGGCAAGGAGGACCTTAAGTTCCCTCGGGGACAGGGCGTGCCA
ATCAACACCAACTCTAGTCCCGACGACCAGATCGGCTATTATAGAAGA
GCCACAAGACGCATCAGAGGTGGCGACGGCAAGATGAAGGACCTGAGC
CCTCGCTGGTACTTTTACTACCTGGGGACCGGCCCTGAAGCCGGCCTG
CCTTACGGCGCCAACAAGGACGGAATCATCTGGGTCGCCACCGAGGGC
GCCCTGAATACCCCTAAGGACCACATCGGCACCAGAAACCCTGCTAAT
AATGCCGCTATCGTGCTGCAGCTGCCTCAGGGCACCACCCTGCCTAAG
GGCTTCTACGCCGAGGGCTCCCGGGGAGGTTCCCAGGCTAGCAGCAGA
TCTTCCAGCCGGAGCAGAAACAGCTCCAGGAACAGCACACCTGGCAGC
AGCAGAGGTACGAGCCCTGCCCGGATGGCCGGAAACGGCGGCGATGCC
GCCCTGGCCCTGCTGCTGCTGGACAGACTGAACCAGCTCGAGAGCAAG
ATGTCTGGCAAGGGCCAGCAGCAGCAGGGCCAGACAGTGACCAAGAAA
TCCGCCGCTGAGGCCAGCAAAAAACCCAGACAGAAAAGAACCGCTACA
AAGGCCTACAACGTTACCCAGGCCTTTGGCAGACGGGGCCCAGAGCAG
ACCCAGGGAAACTTCGGCGACCAGGAGCTGATCCGGCAGGGCACGGAC
TACAAGCACTGGCCTCAAATCGCCCAGTTTGCCCCTTCCGCCAGCGCT
TTCTTCGGAATGAGCAGAATCGGCATGGAAGTGACACCTAGTGGCACC
TGGCTGACCTACACCGGTGCCATTAAGCTGGATGACAAGGACCCCAAC
TTCAAGGATCAGGTGATCCTGCTGAACAAGCACATTGATGCTTACAAG
ACCTTCCCACCTACCGAGCCAAAAAAAGATAAGAAGAAAAAAGCCGAT
GAGACACAAGCCCTGCCCCAGAGGCAGAAGAAGCAACAAACCGTCACC
CTGCTGCCTGCTGCCGACCTGGACGACTTCAGCAAACAGCTGCAGCAG
AGCATGAGCTCTGCTGATAGCACCCAGGCCATGGCCTCTCCAGCCGCT
CCCAGAGCTGTGTCCTTCGCCGATAATAACGACATCACAAACACCAAC
CTGAGCCGGGGCAGAGGCAGAAACCCTAAACCTAGAGCCGCCCCCAAC
AACACCGTGAGCTGGTATACAGGCCTCACCCAGCATGGCAAGGTGCCT
CTGACATTCCCCCCTGGGCAGGGCGTGCCCCTGAACGCCAACAGCACC
CCTGCCCAGAATGCCGGCTACTGGCGGAGGCAGGACAGAAAGATCAAC
ACTGGTAACGGCATCAAGCAGCTGGCCCCACGGTGGTATTTCTACTAC
ACCGGCACCGGCCCGGAAGCCGCCCTCCCCTTCAGAGCCGTGAAGGAC
GGCATCGTGTGGGTGCACGAGGACGGCGCCACAGATGCCCCGTCTACA
TTTGGCACTCGGAATCCCAATAACGACAGCGCCATCGTGACCCAGTTC
GCCCCTGGCACCAAGCTGCCTAAGAACTTTCACATCGAGGGCACAGGA
GGCAACAGCCAGAGCAGCAGCCGGGCTTCGAGCCTGTCTCGGAATAGC
TCCCGGTCCAGCTCTCAGGGCAGCCGCAGTGGAAATTCCACCCGGGGC
ACATCTCCTGGCCCCAGCGGCATCGGCGCTGTGGGCGGAGACCTGCTC
TACCTGGACCTGCTGAACAGACTGCAGGCACTTGAAAGCGGCAAAGTT
AAGCAATCTCAACCTAAGGTGATCACCAAAAAGGACGCCGCCGCCGCT
AAGAACAAGATGAGACACAAGAGAACAAGCACAAAGAGCTTCAACATG
GTGCAAGCCTTCGGCCTGCGGGGACCTGGCGACCTGCAGGGCAACTTC
GGCGACCTGCAGCTGAACAAGCTGGGCACAGAGGATCCTCGATGGCCC
CAGATCGCCGAACTAGCTCCAACCGCCAGCGCCTTCATGGGCATGAGC
CAGTTCAAGCTGACACACCAGAACAATGACGATCACGGAAATCCTGTG
TACTTCCTGAGATACAGCGGCGCCATCAAGCTGGATCCTAAGAACCCC
AACTACAACAAGTGGCTGGAACTGCTGGAACAGAACATCGACGCCTAC
AAGACCTTCCCCAAGAAGGAAAAGAAGCAGAAGGCCCCTAAAGAGGAA
AGCACAGATCAGATGAGCGAGCCTCCCAAGGAACAGAGAGTGCAGGGA
TCTATCACCCAGAGAACAAGAACAAGACCCAGCGTGCAGCCTGGCCCT
ATGATTGACGTGAACACCGACTAG
>G5004 (SARS-COV-2_N, protein)
SEQ ID NO : 5
MSDNGPQNQRNAPRITFGGPSDSTGSNQNGERSGARSKQRRPQGLPNN
TASWFTALTQHGKEDLKFPRGQGVPINTNSSPDDQIGYYRRATRRIRG
GDGKMKDLSPRWYFYYLGTGPEAGLPYGANKDGIIWVATEGALNTPKD
HIGTRNPANNAAIVLQLPQGTTLPKGFYAEGSRGGSQASSRSSSRSRN
SSRNSTPGSSRGTSPARMAGNGGDAALALLLLDRLNQLESKMSGKGQQ
QQGQTVTKKSAAEASKKPRQKRTATKAYNVTQAFGRRGPEQTQGNFGD
QELIRQGTDYKHWPQIAQFAPSASAFFGMSRIGMEVTPSGTWLTYTGA
IKLDDKDPNFKDQVILLNKHIDAYKTFPPTEPKKDKKKKADETQALPQ
RQKKQQTVTLLPAADLDDESKQLQQSMSSADSTQA
>G5005 (CD5-SP_SARS-COV-2_N, synthetic protein)
SEQ ID NO : 6
MPMGSLQPLATLYLLGMLVASCLGSDNGPQNQRNAPRITFGGPSDSTG
SNQNGERSGARSKQRRPQGLPNNTASWFTALTQHGKEDLKFPRGQGVP
INTNSSPDDQIGYYRRATRRIRGGDGKMKDLSPRWYFYYLGTGPEAGL
PYGANKDGIIWVATEGALNTPKDHIGTRNPANNAAIVLQLPQGTTLPK
GFYAEGSRGGSQASSRSSSRSRNSSRNSTPGSSRGTSPARMAGNGGDA
ALALLLLDRLNQLESKMSGKGQQQQGQTVTKKSAAEASKKPRQKRTAT
KAYNVTQAFGRRGPEQTQGNFGDQELIRQGTDYKHWPQIAQFAPSASA
FFGMSRIGMEVTPSGTWLTYTGAIKLDDKDPNFKDQVILLNKHIDAYK
TFPPTEPKKDKKKKADETQALPQRQKKQQTVTLLPAADLDDFSKQLQQ
SMSSADSTQA
>G5006a (CD5-SP_SARS2-N_MERS-N, synthetic protein)
SEQ ID NO: 7
MPMGSLQPLATLYLLGMLVASCLGSDNGPQNQRNAPRITFGGPSDSTG
SNQNGERSGARSKQRRPQGLPNNTASWFTALTQHGKEDLKFPRGQGVP
INTNSSPDDQIGYYRRATRRIRGGDGKMKDLSPRWYFYYLGTGPEAGL
PYGANKDGIIWVATEGALNTPKDHIGTRNPANNAAIVLQLPQGTTLPK
GFYAEGSRGGSQASSRSSSRSRNSSRNSTPGSSRGTSPARMAGNGGDA
ALALLLLDRLNQLESKMSGKGQQQQGQTVTKKSAAEASKKPRQKRTAT
KAYNVTQAFGRRGPEQTQGNFGDQELIRQGTDYKHWPQIAQFAPSASA
FFGMSRIGMEVTPSGTWLTYTGAIKLDDKDPNFKDQVILLNKHIDAYK
TFPPTEPKKDKKKKADETQALPQRQKKQQTVTLLPAADLDDESKQLQQ
SMSSADSTQAMASPAAPRAVSFADNNDITNTNLSRGRGRNPKPRAAPN
NTVSWYTGLTQHGKVPLTFPPGQGVPLNANSTPAQNAGYWRRQDRKIN
TGNGIKQLAPRWYFYYTGTGPEAALPFRAVKDGIVWVHEDGATDAPST
FGTRNPNNDSAIVTQFAPGTKLPKNFHIEGTGGNSQSSSRASSLSRNS
SRSSSQGSRSGNSTRGTSPGPSGIGAVGGDLLYLDLLNRLQALESGKV
KQSQPKVITKKDAAAAKNKMRHKRTSTKSFNMVQAFGLRGPGDLQGNF
GDLQLNKLGTEDPRWPQIAELAPTASAFMGMSQFKLTHQNNDDHGNPV
YFLRYSGAIKLDPKNPNYNKWLELLEQNIDAYKTFPKKEKKQKAPKEE
STDQMSEPPKEQRVQGSITQRTRTRPSVQPGPMIDVNTD
>human CD5 Signal Peptide (Homo sapiens, protein)
SEQ ID NO : 8
MPMGSLQPLATLYLLGMLVASCLG
>MERS-N protein
SEQ ID NO : 9
MASPAAPRAVSFADNNDITNTNLSRGRGRNPKPRAAPNNTVSWYTGLT
QHGKVPLTFPPGQGVPLNANSTPAQNAGYWRRQDRKINTGNGIKQLAP
RWYFYYTGTGPEAALPFRAVKDGIVWVHEDGATDAPSTFGTRNPNNDS
AIVTQFAPGTKLPKNFHIEGTGGNSQSSSRASSLSRNSSRSSSQGSRS
GNSTRGTSPGPSGIGAVGGDLLYLDLLNRLQALESGKVKQSQPKVITK
KDAAAAKNKMRHKRTSTKSFNMVQAFGLRGPGDLQGNFGDLQLNKLGT
EDPRWPQIAELAPTASAFMGMSQFKLTHQNNDDHGNPVYFLRYSGAIK
LDPKNPNYNKWLELLEQNIDAYKTFPKKEKKQKAPKEESTDQMSEPPK
EQRVQGSITQRTRTRPSVQPGPMIDVNTD
>SARS-COV-1-N protein
SEQ ID NO: 10
MSDNGPQSNQRSAPRITFGGPTDSTDNNQNGGRNGARPKQRRPQGLPN
NTASWFTALTQHGKEELRFPRGQGVPINTNSGPDDQIGYYRRATRRVR
GGDGKMKELSPRWYFYYLGTGPEASLPYGANKEGIVWVATEGALNTPK
DHIGTRNPNNNAATVLQLPQGTTLPKGFYAEGSRGGSQASSRSSSRSR
GNSRNSTPGSSRGNSPARMASGGGETALALLLLDRLNQLESKVSGKGQ
QQQGQTVTKKSAAEASKKPRQKRTATKQYNVTQAFGRRGPEQTQGNFG
DQDLIRQGTDYKHWPQIAQFAPSASAFFGMSRIGMEVTPSGTWLTYHG
AIKLDDKDPQFKDNVILLNKHIDAYKTFPPTEPKKDKKKKTDEAQPLP
QRQKKQPTVTLLPAADMDDESRQLQNSMSGASADSTQA
>cloning site (synthetic DNA)
SEQ ID NO: 11
GGCGCGCCattggccaccGCGGCCGC
The gene of interest (e.g., betacoronavirus
nucleoprotein open reading frame) is inserted
between nucleotides 18 and 19 of SEQ ID NO: 11.
GGCGCGCC is an AscI restriction
site and GCGGCCGC is a NotI restriction site.
>artificial
SEQ ID NO: 12
Attggccacc
(synthetic DNA)
This nucleotide sequence is added to
the 5′ end of the betacoronavirus
nucleoprotein coding region to provide a
Kozak consensus sequence for initiation
of translation of the mRNA.
>Influenza A virus (H5N8) nucleocapsid protein
SEQ ID NO: 13
ASQGTKRSYEQMETGGERQNATEIRASVGRMVGGIGRFYIQMCTELKL
SDYEGRLIQNSITIERMVLSAFDERRNKYLEEHPSAGKDPKKTGGPIY
RRRDGKWVRELILYDKEEIRRIWRQANNGEDATAGLTHLMIWHSNLND
ATYQRTRALVRTGMDPRMCSLMQGSTLPRRSGAAGAAVKGVGTMVMEL
IRMIKRGINDRNFWRGENGRRTRIAYERMCNILKGKFQTAAQRAMMDQ
VRESRNPGNAEIEDLIFLARSALILRGSVAHKSCLPACVYGLAVASGY
DFEREGYSLVGIDPFRLLQNSQVESLIRPNENPAHKSQLVWMACHSAA
FEDLRVSSFIRGTRVVPRGQLSTRGVQIASNENMETMDSSTLELRSRY
WAIRTRSGGTTNQQRASAGQISVQPTFSVQRNLPFERATIMAAFTGNT
EGRTSDMRTEIIRMMESAKPEDVSFQGRGVFELSDEKATNPIVPSFDM
SNEGSYFFGDNAEEYDN
>
>Influenza B virus nucleocapsid protein
SEQ ID NO: 14
MSNMDIDGINTGTIDKTPEEITPGTSGTTRPIIRPATLAPPSNKRTRN
PSPERATTSSEDDVGRKTQKKQTPTEIKKSVYNMVVKLGEFYNQMMVK
AGLNDDMERNLIQNAHAVERILLAATDDKKTEFQKKKNARDVKEGKEE
IDHNKTGGTFYKMVRDDKTIYFSPIRITFLKEEVKTMYKTTMGSDGES
GLNHIMIGHSQMNDVCFQRSKALKRVGLDPSLISTFAGSTIPRRSGAT
GVAIKGGGTLVAEAIRFIGRAMADRGLLRDIKAKTAYEKILLNLKNKC
SAPQQKALVDQVIGSRNPGIADIEDLTLLARSMVVVRPSVASKVVLPI
SIYAKIPQLGFNVEEYSMVGYEAMALYNMATPVSILRMGDDAKDKSQL
FFMSCFGAAYEDLRVLSALTGTEFKPRSALKCKGFHVPAKEQVEGMGA
ALMSIKLQFWAPMTRSGGNEVGGDGGSGQISCSPVFAVERPIALSKQA
VRRMLSMNIEGRDADVKGNLLKMMNDSMAKKTSGNAFIGKKMFQISDK
NKTNPVEIPIKQTIPNFFFGRDTAEDYDDLDY
>G5010 (synthetic mRNA)
[codon-optimized_sequence_for_Pan-
Influenza_Antigen]
SEQ ID NO: 15
ATGCCTATGGGCAGCCTGCAGCCACTGGCTACACTGTACCTGCTGGGC
ATGCTGGTGGCCTCTTGTCTGGGCGCCAGCCAAGGCACTAAGAGAAGC
TACGAGCAGATGGAAACCGGAGGCGAACGGCAGAACGCCACAGAGATC
AGAGCCTCTGTGGGCCGTATGGTCGGCGGCATCGGCAGATTCTACATC
CAGATGTGCACCGAACTGAAGCTGAGCGACTACGAGGGCCGCCTGATC
CAGAACAGCATCACAATCGAGAGAATGGTGCTGTCCGCCTTTGACGAG
CGGAGAAACAAATACCTGGAAGAGCACCCTAGCGCCGGAAAAGATCCT
AAGAAAACCGGCGGACCTATCTACAGAAGAAGAGATGGTAAGTGGGTG
AGAGAGCTGATTCTGTACGATAAGGAAGAGATTCGAAGAATCTGGAGA
CAGGCCAACAACGGCGAGGATGCCACCGCAGGCCTGACACACCTGATG
ATCTGGCACAGCAACCTGAACGATGCGACCTACCAGCGCACGCGGGCC
CTGGTCAGAACCGGCATGGATCCTCGGATGTGTAGCCTGATGCAGGGC
AGCACACTGCCAAGACGGAGTGGGGCCGCCGGCGCTGCAGTGAAGGGC
GTCGGAACCATGGTGATGGAGCTGATCCGGATGATAAAGCGGGGCATC
AACGACAGAAACTTCTGGCGAGGCGAGAACGGCCGAAGAACCCGGATC
GCCTACGAGAGAATGTGCAACATCCTGAAAGGAAAATTCCAGACCGCC
GCCCAGCGGGCCATGATGGACCAGGTGCGCGAGAGCAGAAACCCCGGC
AATGCCGAGATCGAGGACCTGATCTTCCTGGCCAGAAGCGCCCTCATT
CTTAGAGGCTCTGTGGCCCACAAGAGCTGTCTGCCTGCCTGTGTGTAC
GGCCTGGCAGTGGCCTCAGGCTACGACTTCGAGCGGGAAGGATACAGT
CTGGTGGGCATCGACCCTTTCAGACTCCTGCAGAATAGCCAGGTGTTT
AGCCTGATCAGACCAAACGAAAACCCCGCCCATAAGAGCCAGCTGGTG
TGGATGGCCTGCCACAGCGCCGCCTTTGAGGATCTGAGAGTGAGCTCT
TTTATCAGAGGCACCCGGGTGGTTCCACGAGGTCAACTGTCTACAAGA
GGTGTGCAGATCGCCAGCAACGAGAACATGGAGACCATGGATAGCAGC
ACCCTGGAACTGAGATCCAGATACTGGGCCATCAGGACACGGAGCGGC
GGCACCACCAATCAGCAGCGCGCCAGCGCCGGCCAGATCTCTGTCCAG
CCTACGTTTAGCGTGCAGCGGAATTTGCCCTTCGAACGCGCCACAATC
ATGGCTGCTTTCACCGGCAATACAGAGGGCAGAACCAGCGATATGAGA
ACAGAAATTATCCGTATGATGGAGTCCGCAAAACCTGAGGACGTGTCC
TTCCAAGGCAGAGGCGTGTTCGAGCTGAGCGACGAGAAGGCCACCAAC
CCTATCGTGCCTAGCTTCGATATGTCTAATGAGGGCAGCTACTTTTTC
GGAGATAACGCCGAAGAGTACGACAACATGTCTAATATGGATATCGAC
GGCATTAACACCGGCACCATCGACAAAACCCCTGAGGAGATCACCCCT
GGCACCAGCGGCACAACCCGGCCCATCATCCGCCCCGCTACACTGGCT
CCACCTAGCAACAAGCGGACCAGAAATCCCTCGCCAGAAAGAGCCACA
ACCTCCAGCGAGGACGACGTGGGACGGAAGACACAAAAGAAGCAGACC
CCTACAGAGATCAAGAAGTCTGTTTACAACATGGTGGTGAAACTGGGC
GAGTTCTACAACCAGATGATGGTGAAGGCCGGCCTGAACGACGATATG
GAAAGAAATCTGATCCAGAACGCCCACGCCGTGGAGCGGATTCTGCTG
GCCGCCACCGATGATAAGAAGACCGAATTCCAGAAAAAGAAAAACGCC
AGAGACGTGAAGGAAGGCAAGGAAGAGATCGACCACAACAAGACAGGC
GGCACATTCTACAAGATGGTCCGGGACGACAAGACCATCTACTTCAGC
CCTATCCGGATAACATTCCTGAAAGAAGAAGTGAAGACCATGTACAAA
ACCACAATGGGCTCTGACGGCTTCAGCGGCCTGAATCACATCATGATC
GGCCACTCTCAAATGAACGATGTGTGCTTCCAGAGAAGCAAGGCTCTG
AAGCGCGTGGGCCTGGATCCTAGCCTGATCTCTACCTTCGCCGGCAGC
ACCATCCCCAGAAGATCGGGCGCTACCGGCGTGGCTATCAAGGGAGGA
GGCACACTGGTGGCTGAAGCCATCAGATTCATCGGAAGAGCCATGGCC
GACAGAGGACTCCTGAGAGATATCAAAGCCAAAACCGCCTACGAAAAA
ATCCTGCTGAACCTGAAGAACAAGTGCAGCGCGCCTCAACAGAAGGCC
CTGGTGGACCAGGTTATCGGCTCTAGAAACCCTGGAATCGCCGATATC
GAGGACCTGACACTGCTGGCCAGATCTATGGTGGTGGTGAGACCCTCC
GTGGCCAGCAAGGTGGTGCTGCCTATCAGCATCTACGCCAAGATCCCT
CAGCTGGGATTTAACGTGGAAGAATACAGCATGGTTGGTTATGAGGCC
ATGGCCCTGTACAACATGGCCACACCTGTGTCCATCCTGAGAATGGGC
GACGATGCCAAAGACAAGAGCCAGCTGTTCTTCATGAGCTGCTTCGGC
GCTGCCTATGAGGACCTGAGAGTGCTGTCCGCTCTTACAGGAACAGAG
TTCAAGCCTAGGAGCGCACTGAAGTGCAAGGGCTTCCACGTGCCCGCC
AAGGAACAGGTGGAAGGCATGGGAGCTGCTCTGATGTCCATCAAGCTG
CAATTTTGGGCTCCTATGACCCGGAGCGGCGGAAATGAGGTGGGTGGC
GACGGAGGCAGCGGACAGATTTCTTGCAGCCCCGTATTTGCCGTGGAG
AGACCAATCGCCCTGTCCAAGCAGGCCGTGAGAAGAATGCTGAGCATG
AACATCGAGGGCCGGGACGCCGACGTGAAGGGCAACCTGTTGAAGATG
ATGAACGACAGCATGGCCAAGAAGACCAGTGGCAATGCCTTCATCGGC
AAGAAGATGTTCCAGATCTCCGACAAGAACAAGACCAACCCCGTGGAA
ATCCCCATCAAGCAGACAATCCCTAACTTCTTCTTCGGCAGAGACACC
GCCGAAGACTATGACGACCTGGACTACTGA
>G5010 (synthetic protein) [Pan-Influenza
Antigen]
SEQ ID NO: 16
MPMGSLQPLATLYLLGMLVASCLGASQGTKRSYEQMETGGERQNATEI
RASVGRMVGGIGRFYIQMCTELKLSDYEGRLIQNSITIERMVLSAFDE
RRNKYLEEHPSAGKDPKKTGGPIYRRRDGKWVRELILYDKEEIRRIWR
QANNGEDATAGLTHLMIWHSNLNDATYQRTRALVRTGMDPRMCSLMQG
STLPRRSGAAGAAVKGVGTMVMELIRMIKRGINDRNFWRGENGRRTRI
AYERMCNILKGKFQTAAQRAMMDQVRESRNPGNAEIEDLIFLARSALI
LRGSVAHKSCLPACVYGLAVASGYDFEREGYSLVGIDPFRLLQNSQVE
SLIRPNENPAHKSQLVWMACHSAAFEDLRVSSFIRGTRVVPRGQLSTR
GVQIASNENMETMDSSTLELRSRYWAIRTRSGGTTNQQRASAGQISVQ
PTFSVQRNLPFERATIMAAFTGNTEGRTSDMRTEIIRMMESAKPEDVS
FQGRGVFELSDEKATNPIVPSFDMSNEGSYFFGDNAEEYDNMSNMDID
GINTGTIDKTPEEITPGTSGTTRPIIRPATLAPPSNKRTRNPSPERAT
TSSEDDVGRKTQKKQTPTEIKKSVYNMVVKLGEFYNQMMVKAGLNDDM
ERNLIQNAHAVERILLAATDDKKTEFQKKKNARDVKEGKEEIDHNKTG
GTFYKMVRDDKTIYFSPIRITFLKEEVKTMYKTTMGSDGESGLNHIMI
GHSQMNDVCFQRSKALKRVGLDPSLISTFAGSTIPRRSGATGVAIKGG
GTLVAEAIRFIGRAMADRGLLRDIKAKTAYEKILLNLKNKCSAPQQKA
LVDQVIGSRNPGIADIEDLTLLARSMVVVRPSVASKVVLPISIYAKIP
QLGENVEEYSMVGYEAMALYNMATPVSILRMGDDAKDKSQLFFMSCFG
AAYEDLRVLSALTGTEFKPRSALKCKGFHVPAKEQVEGMGAALMSIKL
QFWAPMTRSGGNEVGGDGGSGQISCSPVFAVERPIALSKQAVRRMLSM
NIEGRDADVKGNLLKMMNDSMAKKTSGNAFIGKKMFQISDKNKTNPVE
IPIKQTIPNFFFGRDTAEDYDDLDY
>Nucleoprotein of Influenza A virus (H2N2)
(Swiss-Protein ID P21433) [JPT peptide
Product Code: PM-INFA-NPH2N2]
SEQ ID NO: 17
ASQGTKRSYEQMETDGERQNANEIRASVGKMIGGIGRFYIQMCTELKL
SDYEGRLIQNSLTIERMVLSAFDERRNKYLEEHPSAGKDPKKTGGPIY
KRVDGKWMRELVLYDKEEIRRIWRQANNGDDATAGLTHMMIWHSNLND
TTYQRTRALVRTGMDPRMCSLMQGSTLPRRSGAAGAAVKGVGTMVMEL
IRMIKRGINDRNFWRGENGRKTRNAYERMCNILKGKFQTAAQRAMMDQ
VRESRNPGNAEIEDLIFLARSALILRGSVAHKSCLPACVYGPAVASGY
DFEKEGYSLVGIDPFKLLQNSQVYSLIRPNENPAHKSQLVWMACNSAA
FEDLRVSSFIRGTKVIPRGKLSTRGVQIASNENMDTMGSSTLELRSRY
WAIRTRSGGNTNQQRASAGQISVQPTFSVQRNLPEDKPTIMAAFTGNA
EGRTSDMRAEIIRMMEGAKPEEVSFQGRGVFELSDEKATNPIVPSFDM
SNEGSYFFGDNAEEYDN
>Nucleoprotein fragment of Zaire ebolavirus
SEQ ID NO: 18
DSRPQKIWMAPSLTESDMDYHKILTAGLSVQQGIVRQRVIPVYQVNNL
EEICQLIIQAFEAGVDFQESADSFLLMLCLHHAYQGDYKLFLESGAVK
YLEGHGFRFEVKKRDGVKRLEELLPAVSSGKNIKRTLAAMPEEETTEA
NAGQFLSFASLFLPKLVVGEKACLEKVQRQIQVHAEQGLIQYPTAWQS
VGHMMVIFRLMRTNFLIKFLLIHQGMHMVAGHDANDAVISNSVAQARF
SGLLIVKTVLDHILQKTERGVRLHPLARTAKVKNEVNSFKAALSSLAK
HGEYAPFARLLNLSGVNNLEHGLFPQLSAIALGVATAHGSTLAGVNVG
EQYQQLREAATEAEKQLQQYAESRELDHLGLDDQEKKILMNFHQKKNE
ISFQQTNAMVTLRKERLAKLTEAITAASLPKTSGHYDDDDDIPFPGPI
NDDDNPGHQDDDPTDSQDTTIPDVVVDPDDGSYGEYQSYSENGMNAPD
DLVLFDLDEDDEDTKPVPNRSTKGGQQKNSQKGQHIEGRQTQFRPIQN
VPGPHRTIHHASAPLTDNDRRNEPSGSTSPRMLTPINEEADPLDDADD
ETSSLPPLESDDEEQDRDGTSNRTPTVAPPAPVYRDHSEKKELPQDEQ
QDQDHTQEARNQDSDNTQSEHSLEEMYRHILRSQGPFDAVLYYHMMKD
EPVVESTSDGKEYTYPDSLEEEYPPWLTEKEAMNEENRFVTLDGQQFY
WPVMNHKNKEMAILQHHQ
>Nucleoprotein fragment of Sudan ebolavirus
SEQ ID NO: 19
AKLTEAITTASKIKVGDRYPDDNDIPFPGPIYDDTHPNPSDDNPDDSR
DTTIPGGVVDPYDDESNNYPDYEDSAEGTTGDLDLFNLDDDDDDSRPG
PPDRGQNKERAARTYGLQDPTLDGAKKVPELTPGSHQPGNLHITKSGS
NTNQPQGNMSSTLHSMTPIQEESEPDDQKDNDDESLTSLDSEGDEDGE
SISEENTPTVAPPAPVYKDTGVDTNQQNGPSSTVDSQGSESEALPINS
KKSSALEETYYHLLKTQGPFEAINYYHLMSDEPIAFSTESGKEYIFPD
SLEEAYPPWLSEKEALEKENRYLVIDGQQFLWPVMSLRDKFLAVLQHD
>Nucleoprotein fragment of Bundibugyo ebolavirus
SEQ ID NO: 20
AKLTEAITSTSILKTGRRYDDDNDIPFPGPINDNENSGQNDDDPTDSQ
DTTIPDVIIDPNDGGYNNYSDYANDAASAPDDLVLEDLEDEDDADNPA
QNTPEKNDRPATTKLRNGQDQDGNQGETASPRVAPNQYRDKPMPQVQD
RSENHDQTLQTQSRVLTPISEEADPSDHNDGDNESIPPLESDDEGSTD
TTAAETKPATAPPAPVYRSISVDDSVPSENIPAQSNQTNNEDNVRNNA
QSEQSIAEMYQHILKTQGPFDAILYYHMMKEEPIIFSTSDGKEYTYPD
SLEDEYPPWLSEKEAMNEDNRFITMDGQQFYWPVMNHRNKEMAILQHH
R
>Nucleoprotein fragment of Taï Forest ebolavirus
SEQ ID NO: 21
LVLFDLEDGDEDDHRPSSSSENNNKHSLTGTDSNKTSNWNRNPTNMPK
KDSTQNNDNPAQRAQEYARDNIQDTPTPHRALTPISEETGSNGHNEDD
IDSIPPLESDEENNTETTITTTKNTTAPPAPVYRSNSEKEPLPQEKSQ
KQPNQVSGSENTDNKPHSEQSVEEM
>Pan-Ebola_Antigen (synthetic protein)
SEQ ID NO: 22
MPMGSLQPLATLYLLGMLVASCLGDSRPQKIWMAPSLTESDMDYHKIL
TAGLSVQQGIVRQRVIPVYQVNNLEEICQLIIQAFEAGVDFQESADSF
LLMLCLHHAYQGDYKLFLESGAVKYLEGHGFRFEVKKRDGVKRLEELL
PAVSSGKNIKRTLAAMPEEETTEANAGQFLSFASLFLPKLVVGEKACL
EKVQRQIQVHAEQGLIQYPTAWQSVGHMMVIFRLMRTNFLIKFLLIHQ
GMHMVAGHDANDAVISNSVAQARFSGLLIVKTVLDHILQKTERGVRLH
PLARTAKVKNEVNSFKAALSSLAKHGEYAPFARLLNLSGVNNLEHGLF
PQLSAIALGVATAHGSTLAGVNVGEQYQQLREAATEAEKQLQQYAESR
ELDHLGLDDQEKKILMNFHQKKNEISFQQTNAMVTLRKERLAKLTEAI
TAASLPKTSGHYDDDDDIPFPGPINDDDNPGHQDDDPTDSQDTTIPDV
VVDPDDGSYGEYQSYSENGMNAPDDLVLFDLDEDDEDTKPVPNRSTKG
GQQKNSQKGQHIEGRQTQFRPIQNVPGPHRTIHHASAPLTDNDRRNEP
SGSTSPRMLTPINEEADPLDDADDETSSLPPLESDDEEQDRDGTSNRT
PTVAPPAPVYRDHSEKKELPQDEQQDQDHTQEARNQDSDNTQSEHSLE
EMYRHILRSQGPFDAVLYYHMMKDEPVVESTSDGKEYTYPDSLEEEYP
PWLTEKEAMNEENRFVTLDGQQFYWPVMNHKNKEMAILQHHQAKLTEA
ITTASKIKVGDRYPDDNDIPFPGPIYDDTHPNPSDDNPDDSRDTTIPG
GVVDPYDDESNNYPDYEDSAEGTTGDLDLFNLDDDDDDSRPGPPDRGQ
NKERAARTYGLQDPTLDGAKKVPELTPGSHQPGNLHITKSGSNTNQPQ
GNMSSTLHSMTPIQEESEPDDQKDNDDESLTSLDSEGDEDGESISEEN
TPTVAPPAPVYKDTGVDTNQQNGPSSTVDSQGSESEALPINSKKSSAL
EETYYHLLKTQGPFEAINYYHLMSDEPIAFSTESGKEYIFPDSLEEAY
PPWLSEKEALEKENRYLVIDGQQFLWPVMSLRDKFLAVLQHDAKLTEA
ITSTSILKTGRRYDDDNDIPFPGPINDNENSGQNDDDPTDSQDTTIPD
VIIDPNDGGYNNYSDYANDAASAPDDLVLFDLEDEDDADNPAQNTPEK
NDRPATTKLRNGQDQDGNQGETASPRVAPNQYRDKPMPQVQDRSENHD
QTLQTQSRVLTPISEEADPSDHNDGDNESIPPLESDDEGSTDTTAAET
KPATAPPAPVYRSISVDDSVPSENIPAQSNQTNNEDNVRNNAQSEQSI
AEMYQHILKTQGPFDAILYYHMMKEEPIIFSTSDGKEYTYPDSLEDEY
PPWLSEKEAMNEDNRFITMDGQQFYWPVMNHRNKFMAILQHHRLVLFD
LEDGDEDDHRPSSSSENNNKHSLTGTDSNKTSNWNRNPTNMPKKDSTQ
NNDNPAQRAQEYARDNIQDTPTPHRALTPISEETGSNGHNEDDIDSIP
PLESDEENNTETTITTTKNTTAPPAPVYRSNSEKEPLPQEKSQKQPNQ
VSGSENTDNKPHSEQSVEEM
>Pan-Ebola_Antigen (synthetic mRNA)
SEQ ID NO: 23
ATGcccatggggtctctgcaaccgctggccaccttgtacctgctgggg
atgctggtcgcttcctgcctcggagattctcgtcctcagaaaatctgg
atggcgccgagtctcactgaatctgacatggattaccacaagatcttg
acagcaggtctgtccgttcaacaggggattgttcggcaaagagtcatc
ccagtgtatcaagtaaacaatcttgaagaaatttgccaacttatcata
caggcctttgaagcaggtgttgattttcaagagagtgcggacagtttc
cttctcatgctttgtcttcatcatgcgtaccagggagattacaaactt
ttcttggaaagtggcgcagtcaagtatttggaagggcacgggttccgt
tttgaagtcaagaagcgtgatggagtgaagcgccttgaggaattgctg
ccagcagtatctagtggaaaaaacattaagagaacacttgctgccatg
ccggaagaggagacaactgaagctaatgccggtcagtttctctccttt
gcaagtctattccttccgaaattggtagtaggagaaaaggcttgcctt
gagaaggttcaaaggcaaattcaagtacatgcagagcaaggactgata
caatatccaacagcttggcaatcagtaggacacatgatggtgattttc
cgtttgatgcgaacaaattttctgatcaaatttctcctaatacaccaa
gggatgcacatggttgccgggcatgatgccaacgatgctgtgatttca
aattcagtggctcaagctcgtttttcaggcttattgattgtcaaaaca
gtacttgatcatatcctacaaaagacagaacgaggagttcgtctccat
cctcttgcaaggaccgccaaggtaaaaaatgaggtgaactcctttaag
gctgcactcagctccctggccaagcatggagagtatgctcctttcgcc
cgacttttgaacctttctggagtaaataatcttgagcatggtcttttc
cctcaactatcggcaattgcactcggagtcgccacagcacacgggagt
accctcgcaggagtaaatgttggagaacagtatcaacaactcagagag
gctgccactgaggctgagaagcaactccaacaatatgcagagtctcgc
gaacttgaccatcttggacttgatgatcaggaaaagaaaattcttatg
aacttccatcagaaaaagaacgaaatcagcttccagcaaacaaacgct
atggtaactctaagaaaagagcgcctggccaagctgacagaagctatc
actgctgcgtcactgcccaaaacaagtggacattacgatgatgatgac
gacattccctttccaggacccatcaatgatgacgacaatcctggccat
caagatgatgatccgactgactcacaggatacgaccattcccgatgtg
gtggttgatcctgatgatggaagctacggcgaataccagagttactcg
gaaaacggcatgaatgcaccagatgacttggtcctattcgatctagac
gaggacgacgaggacactaagccagtgcctaatagatcgaccaagggt
ggacaacagaagaacagtcaaaagggccagcatatagagggcagacag
acacaattcaggccaattcaaaatgtcccaggccctcacagaacaatc
caccacgccagtgcgccactcacggacaatgacagaagaaatgaaccc
tccggctcaaccagccctcgcatgctgacaccaattaacgaagaggca
gacccactggacgatgccgacgacgagacgtctagccttccgcccttg
gagtcagatgatgaagagcaggacagggacggaacttccaaccgcaca
cccactgtcgccccaccggctcccgtatacagagatcactctgaaaag
aaagaactcccgcaagacgagcaacaagatcaggaccacactcaagag
gccaggaaccaggacagtgacaacacccagtcagaacactcccttgag
gagatgtatcgccacattctaagatcacaggggccatttgatgctgtt
ttgtattatcatatgatgaaggatgagcctgtagttttcagtaccagt
gatggcaaagagtacacgtatccagactcccttgaagaggaatatcca
ccatggctcactgaaaaagaggctatgaatgaagagaatagatttgtt
acattggatggtcaacaattttattggccggtgatgaatcacaagaat
aaattcatggcaatcctgcaacatcatcaggctaaattgaccgaagcc
atcacgactgcatcgaagatcaaggttggagaccgttatcctgatgac
aatgatattccatttcccgggccgatctatgatgacactcaccccaat
ccctctgatgacaatcctgatgattcacgtgatacaactattccaggt
ggtgttgttgacccgtatgatgatgagagtaataattatcctgactac
gaggattcggctgaaggcaccacaggagatcttgatctcttcaatttg
gacgacgacgatgatgacagccgaccaggaccaccagacagggggcag
aacaaggagagggcggcccggacatatggcctccaagatccgaccttg
gacggagcgaaaaaggtgccggagttgaccccaggttcccatcaacca
ggcaacctccacatcaccaagtcgggttcaaacaccaaccaaccacaa
ggcaatatgtcatctactctccatagtatgacccctatacaggaagaa
tcagagcccgatgatcaaaaagataatgatgacgagagtctcacatcc
cttgactctgaaggtgacgaagatggtgagagcatctctgaggagaac
accccaactgtagctccaccagcaccagtctacaaagacactggagta
gacactaatcagcagaatggaccaagcagtactgtagatagtcaaggt
tctgaaagtgaagctctcccaatcaactctaaaaagagttccgcacta
gaagaaacatattatcatctcctaaaaacacagggtccatttgaggca
atcaattattatcacctaatgagtgatgaacccattgcttttagcact
gaaagtggcaaggaatatatctttccagactcccttgaagaagcctac
ccgccgtggttgagtgagaaggaggccttagagaaggaaaatcgttat
ctggtcattgatggccagcaattcctctggccggtaatgagcctacgg
gacaagttccttgccgttcttcaacatgacgccaaattgaccgaagct
attacttccacctctatcctcaaaacaggaaggcggtatgatgatgac
aatgacataccctttccagggccaatcaatgataacgagaactctggt
cagaacgatgacgatccaacagactcccaggataccacaatcccggat
gtaataatcgatccaaacgatggtgggtataataattacagcgattat
gcaaatgatgctgcaagtgctcctgatgacctagttctttttgacctt
gaggacgaggatgatgctgataacccggctcaaaacacgccagaaaaa
aatgatagaccagcaacaacaaagctgagaaatggacaggaccaggat
ggaaaccaaggcgaaactgcatccccacgggtagcccccaaccaatac
agagacaagccaatgccacaagtacaggacagatccgaaaatcatgac
caaacccttcaaacacagtccagggttttgactcctatcagcgaggaa
gcagaccccagcgaccacaacgatggtgacaatgaaagcattcctccc
ctggaatcagacgacgagggtagcactgatactactgcagcagaaaca
aagcctgccactgcacctcccgctcccgtctaccgaagtatctccgta
gatgattctgtcccctcagagaacattcccgcacagtccaatcaaacg
aacaatgaggacaatgtcaggaacaatgctcagtcggagcaatccatt
gcagaaatgtatcaacatatcttgaaaacacaaggaccttttgatgcc
atcctttactaccatatgatgaaagaagagcccatcattttcagcact
agtgatgggaaggagtatacatatccagactctcttgaagatgagtat
ccaccctggctcagcgagaaggaagccatgaacgaagacaatagattc
ataaccatggatggtcagcagttttactggcctgtgatgaatcataga
aataaattcatggcaatcctccagcatcacaggcttgttctttttgac
cttgaagatggtgacgaggatgatcaccgaccgtcaagttcatcagag
aacaacaacaaacacagtcttacaggaactgacagtaacaaaacaagt
aactggaatcgaaacccgactaatatgccaaagaaagactccacacaa
aacaatgacaatcctgcacagcgggctcaagaatacgccagggataac
atccaggatacaccaacaccccatcgagctctaactcccatcagcgaa
gaaaccggctccaatggtcacaatgaagatgacattgatagcatccct
cctttggaatcagacgaagaaaacaacactgagacaaccattaccacc
acaaaaaataccactgctccaccagcacctgtttatcggagtaattca
gaaaaggagcccctcccgcaagaaaaatcccagaagcaaccaaaccaa
gtgagtggtagtgagaataccgacaataaacctcactcagagcaatca
gtggaagaaatgTAA
>G5003o (synthetic mRNA) [CD5sp + RBD2 (Omicron)]
SEQ ID NO: 24
ATGcccatggggtctctgcaaccgctggccaccttgtacctgctgggg
atgctggtcgcttcctgcctcggaagagtccaaccaacagaatctatt
gttagatttcctaatattacaaacttgtgcccttttgatgaagttttt
aacgccaccagatttgcatctgtttatgcttggaacaggaagagaatc
agcaactgtgttgctgattattctgtcctatataatctcgcaccattt
ttcacttttaagtgttatggagtgtctcctactaaattaaatgatctc
tgctttactaatgtctatgcagattcatttgtaattagaggtgatgaa
gtcagacaaatcgctccagggcaaactggaaacattgctgattataat
tataaattaccagatgattttacaggctgcgttatagcttggaattct
aacaagcttgattctaaggttagtggtaattataattacctgtataga
ttgtttaggaagtctaatctcaaaccttttgagagagatatttcaact
gaaatctatcaggccggtaacaaaccttgtaatggtgttgcaggtttt
aattgttactttcctttacgatcatatagtttccgacccacttatggt
gttggtcaccaaccatacagagtagtagtactttcttttgaacttcta
catgcaccagcaactgtttgtggacctaaaaagtctactaatttggtt
aaaaacaaatgtgtcaatttcTAA
>G5003o (synthetic protein) [CD5sp + RBD2
(Omicron)]
SEQ ID No: 25
MPMGSLQPLATLYLLGMLVASCLGRVQPTESIVRFPNITNLCPFDEVE
NATRFASVYAWNRKRISNCVADYSVLYNLAPFFTFKCYGVSPTKLNDL
CFTNVYADSFVIRGDEVRQIAPGQTGNIADYNYKLPDDFTGCVIAWNS
NKLDSKVSGNYNYLYRLFRKSNLKPFERDISTEIYQAGNKPCNGVAGE
NCYFPLRSYSFRPTYGVGHQPYRVVVLSFELLHAPATVCGPKKSTNLV
KNKCVNF
>G5006d (synthetic mRNA)
SEQ ID No: 26
atgcccatggggtctctgcaaccgctggccaccttgtacctgctgggg
atgctggtcgcttcctgcctcggaagagtccaaccaacagaatctatt
gttagatttcctaatattacaaacttgtgcccttttggtgaagttttt
aacgccaccagatttgcatctgtttatgcttggaacaggaagagaatc
agcaactgtgttgctgattattctgtcctatataattccgcatcattt
tccacttttaagtgttatggagtgtctcctactaaattaaatgatctc
tgctttactaatgtctatgcagattcatttgtaattagaggtgatgaa
gtcagacaaatcgctccagggcaaactggaaagattgctgattataat
tataaattaccagatgattttacaggctgcgttatagcttggaattct
aacaatcttgattctaaggttggtggtaattataattaccggtataga
ttgtttaggaagtctaatctcaaaccttttgagagagatatttcaact
gaaatctatcaggccggtagcaaaccttgtaatggtgttgaaggtttt
aattgttactttcctttacaatcatatggtttccaacccactaatggt
gttggttaccaaccatacagagtagtagtactttcttttgaacttcta
catgcaccagcaactgtttgtggacctaaaaagtctactaatttggtt
aaaaacaaatgtgtcaatttcTCAGGTGGTGGCGGTTCAGGCGGAGGT
GGCTCTGGCGGTGGCGGATCGatgtctgataatggaccccaaaatcag
cgaaatgcaccccgcattacgtttggtggaccctcagattcaactggc
agtaaccagaatggagaacgcagtggggcgcgatcaaaacaacgtcgg
ccccaaggtttacccaataatactgcgtcttggttcaccgctctcact
caacatggcaaggaaggccttaaattccctcgaggacaaggcgttcca
attaacaccaatagcagtccagatgaccaaattggctactaccgaaga
gctaccagacgaattcgtggtggtgacggtaaaatgaaagatctcagt
ccaagatggtatttctactacctaggaactgggccagaagctggactt
ccctatggtgctaacaaagacggcatcatatgggttgcaactgaggga
gccttgaatacaccaaaagatcacattggcacccgcaatcctgctaac
aatgctgcaatcgtgctacaacttcctcaaggaacaacattgccaaaa
ggcttctacgcagaagggagcagaggcggcagtcaagcctcttctcgt
tcctcatcacgtagtcgcaacagttcaagaaattcaactccaggcagc
agtatgggaacttctcctgctagaatggctggcaatggctgtgatgct
gctcttgctttgctgctgcttgacagattgaaccagcttgagagcaaa
atgtctggtaaaggccaacaacaacaaggccaaactgtcactaagaaa
tctgctgctgaggcttctaagaagcctcggcaaaaacgtactgccact
aaagcatacaatgtaacacaagctttcggcagacgtggtccagaacaa
acccaaggaaattttggggaccaggaactaatcagacaaggaactgat
tacaaacattggccgcaaattgcacaatttgcccccagcgcttcagcg
ttcttcggaatgtcgcgcattggcatggaagtcacaccttcgggaacg
tggttgacctacacaggtgccatcaaattggatgacaaagatccaaat
ttcaaagatcaagtcattttgctgaataagcatattgacgcatacaaa
acattcccaccaacagagcctaaaaaggacaaaaagaagaaggcttat
gaaactcaagccttaccgcagagacagaagaaacagcaaactgtgact
cttcttcctgctgcagatttggatgatttctccaaacaattgcaacaa
tccatgagcagtgctgactcaactcaggccATGGCATCCCCTGCTGCA
CCTCGTGCTGTTTCCTTTGCCGATAACAATGATATAACAAATACAAAC
CTATCTCGAGGTAGAGGACGTAATCCAAAACCACGAGCTGCACCAAAT
AACACTGTCTCTTGGTACACTGGGCTTACCCAACACGGGAAAGTCCCT
CTTACCTTTCCACCTGGGCAGGGTGTACCTCTTAATGCCAATTCTACC
CCTGCGCAAAATGCTGGGTATTGGCGGAGACAGGACAGAAAAATTAAT
ACCGGGAATGGAATTAAGCAACTGGCTCCCAGGTGGTACTTCTACTAC
ACTGGAACTGGACCCGAAGCAGCACTCCCATTCCGGGCTGTTAAGGAT
GGCATCGTTTGGGTCCATGAAGATGGCGCCACTGATGCTCCTTCAACT
TTTGGGACGCGGAACCCTAACAATGATTCAGCTATTGTTACACAATTC
GCGCCCGGTACTAAGCTTCCTAAAAACTTCCACATTGAGGGGACTGGA
GGCAATAGTCAATCATCTTCAAGAGCCTCTAGCTTAAGCAGAAACTCT
TCCAGATCTAGTTCACAAGGTTCAAGATCAGGAAACTCTACCCGCGGC
ACTTCTCCAGGTCCATCTGGAATCGGAGCAGTAGGAGGTGATCTACTT
TACCTTGATCTTCTGAACAGACTACAAGCCCTTGAGTCTGGCAAAGTA
AAGCAATCGCAGCCAAAAGTAATCACTAAGAAAGATGCTGCTGCTGCT
AAAAATAAGATGCGCCACAAGCGCACTTCCACCAAAAGTTTCAACATG
GTGCAAGCTTTTGGTCTTCGCGGACCAGGAGACCTCCAGGGAAACTTT
GGTGATCTTCAATTGAATAAACTCGGCACTGAGGACCCACGTTGGCCC
CAAATTGCTGAGCTTGCTCCTACAGCCAGTGCTTTTATGGGTATGTCG
CAATTTAAACTTACCCATCAGAACAATGATGATCATGGCAACCCTGTG
TACTTCCTTCGGTACAGTGGAGCCATTAAACTTGACCCAAAGAATCCC
AACTACAATAAGTGGTTGGAGCTTCTTGAGCAAAATATTGATGCCTAC
AAAACCTTCCCTAAGAAGGAAAAGAAACAAAAGGCACCAAAAGAAGAA
TCAACAGACCAAATGTCTGAACCTCCAAAGGAGCAGCGTGTGCAAGGT
AGCATCACTCAGCGCACTCGCACCCGTCCAAGTGTTCAGCCTGGTCCA
ATGATTGATGTTAACACTGATTCTGGTGGCGGTGGCTCGGGCGGAGGT
GGGTCGGGTGGCGGCGGATCAgaagcaaaaccttctggctcagttgtg
gaacaggctgaaggtgttgaatgtgatttttcacctcttctgtctggc
acacctcctcaggtttataatttcaagcgtttggtttttaccaattgc
aattataatcttaccaaattgctttcacttttttctgtgaatgatttt
acttgtagtcaaatatctccagcagcaattgctagcaactgttattct
tcactgattttggattacttttcatacccacttagtatgaaatccgat
ctcagtgttagttctgctggtccaatatcccagtttaattataaacag
tccttttctaatcccacatgtttgattttagcgactgttcctcataac
cttactactattactaagcctcttaagtacagctatattaacaagtgc
tctcgtcttctttctgatgatcgtactgaagtacctcagttagtgaac
gctaatcaatactcaccctgtgtatccattgtcccatccactgtgtgg
gaagacggtgattattataggaaacaactatctccacttgaaggtggt
ggctggcttgttgctagtggctcaactgttgccatgactgagcaatta
cagatgggctttggtattacagttcaatatggtacagacaccaatagt
gtttgccccaagcttgaatttgctaatgacacaaaaattgcctctcaa
ttaggcaattgcgtggaatatTAG
>G5006d (synthetic protein)
SEQ ID No: 27
MPMGSLQPLATLYLLGMLVASCLGRVQPTESIVRFPNITNLCPFGEVE
NATRFASVYAWNRKRISNCVADYSVLYNSASFSTFKCYGVSPTKLNDL
CFTNVYADSFVIRGDEVRQIAPGQTGKIADYNYKLPDDFTGCVIAWNS
NNLDSKVGGNYNYRYRLFRKSNLKPFERDISTEIYQAGSKPCNGVEGF
NCYFPLQSYGFQPTNGVGYQPYRVVVLSFELLHAPATVCGPKKSTNLV
KNKCVNFSGGGGSGGGGSGGGGSMSDNGPQNQRNAPRITFGGPSDSTG
SNQNGERSGARSKQRRPQGLPNNTASWFTALTQHGKEGLKFPRGQGVP
INTNSSPDDQIGYYRRATRRIRGGDGKMKDLSPRWYFYYLGTGPEAGL
PYGANKDGIIWVATEGALNTPKDHIGTRNPANNAAIVLQLPQGTTLPK
GFYAEGSRGGSQASSRSSSRSRNSSRNSTPGSSMGTSPARMAGNGCDA
ALALLLLDRLNQLESKMSGKGQQQQGQTVTKKSAAEASKKPRQKRTAT
KAYNVTQAFGRRGPEQTQGNFGDQELIRQGTDYKHWPQIAQFAPSASA
FFGMSRIGMEVTPSGTWLTYTGAIKLDDKDPNEKDQVILLNKHIDAYK
TFPPTEPKKDKKKKAYETQALPQRQKKQQTVTLLPAADLDDFSKQLQQ
SMSSADSTQAMASPAAPRAVSFADNNDITNTNLSRGRGRNPKPRAAPN
NTVSWYTGLTQHGKVPLTFPPGQGVPLNANSTPAQNAGYWRRQDRKIN
TGNGIKQLAPRWYFYYTGTGPEAALPFRAVKDGIVWVHEDGATDAPST
FGTRNPNNDSAIVTQFAPGTKLPKNFHIEGTGGNSQSSSRASSLSRNS
SRSSSQGSRSGNSTRGTSPGPSGIGAVGGDLLYLDLLNRLQALESGKV
KQSQPKVITKKDAAAAKNKMRHKRTSTKSFNMVQAFGLRGPGDLQGNF
GDLQLNKLGTEDPRWPQIAELAPTASAFMGMSQFKLTHQNNDDHGNPV
YFLRYSGAIKLDPKNPNYNKWLELLEQNIDAYKTFPKKEKKQKAPKEE
STDQMSEPPKEQRVQGSITQRTRTRPSVQPGPMIDVNTDSGGGGSGGG
GSGGGGSEAKPSGSVVEQAEGVECDFSPLLSGTPPQVYNFKRLVFTNC
NYNLTKLLSLFSVNDFTCSQISPAAIASNCYSSLILDYFSYPLSMKSD
LSVSSAGPISQFNYKQSFSNPTCLILATVPHNLTTITKPLKYSYINKC
SRLLSDDRTEVPQLVNANQYSPCVSIVPSTVWEDGDYYRKQLSPLEGG
GWLVASGSTVAMTEQLQMGFGITVQYGTDTNSVCPKLEFANDTKIASQ
LGNCVEY
>G50012 (synthetic mRNA)
SEQ ID No: 28
atgcctatgggcagcctgcagccactggctacactgtacctgctgggc
atgctggtggcctcttgtctgggcataggagagtgtccaaagtatgtc
aggagtgcaaaattaaggatggttacaggactaaggaacatcccatcc
attcaatccagaggtttgtttggagccattgccggtttcattgaaggg
gggtggactggaatggtagatgggtggtatggttatcatcatcagaat
gagcaaggatctggctatgctgcagatcaaaaaagtacacaaaatgcc
attaacgggattacaaacaaggtgaattctgtaattgagaaaatgaac
actcaattcacagctgtgggcaaagaattcaacaaattggaaagaagg
atggaaaacttaaataaaaaagttgatgatgggtttctagacatttgg
acatataatgcagaattgttggttctactggaaaatgaaaggactttg
gatttccatgactccgccagccaaggcactaagagaagctacgagcag
atggaaaccggaggcgaacggcagaacgccacagagatcagagcctct
gtgggccgtatggtcggcggcatcggcagattctacatccagatgtgc
accgaactgaagctgagcgactacgagggccgcctgatccagaacagc
atcacaatcgagagaatggtgctgtccgcctttgacgagcggagaaac
aaatacctggaagagcaccctagcgccggaaaagatcctaagaaaacc
ggcggacctatctacagaagaagagatggtaagtgggtgagagagctg
attctgtacgataaggaagagattcgaagaatctggagacaggccaac
aacggcgaggatgccaccgcaggcctgacacacctgatgatctggcac
agcaacctgaacgatgcgacctaccagcgcacgcgggccctggtcaga
accggcatggatcctcggatgtgtagcctgatgcagggcagcacactg
ccaagacggagtggggccgccggcgctgcagtgaagggcgtcggaacc
atggtgatggagctgatccggatgataaagcggggcatcaacgacaga
aacttctggcgaggcgagaacggccgaagaacccggatcgcctacgag
agaatgtgcaacatcctgaaaggaaaattccagaccgccgcccagcgg
gccatgatggaccaggtgcgcgagagcagaaaccccggcaatgccgag
atcgaggacctgatcttcctggccagaagcgccctcattcttagaggc
tctgtggcccacaagagctgtctgcctgcctgtgtgtacggcctggca
gtggcctcaggctacgacttcgagcgggaaggatacagtctggtgggc
atcgaccctttcagactcctgcagaatagccaggtgtttagcctgatc
agaccaaacgaaaaccccgcccataagagccagctggtgtggatggcc
tgccacagcgccgcctttgaggatctgagagtgagctcttttatcaga
ggcacccgggtggttccacgaggtcaactgtctacaagaggtgtgcag
atcgccagcaacgagaacatggagaccatggatagcagcaccctggaa
ctgagatccagatactgggccatcaggacacggagcggcggcaccacc
aatcagcagcgcgccagcgccggccagatctctgtccagcctacgttt
agcgtgcagcggaatttgcccttcgaacgcgccacaatcatggctgct
ttcaccggcaatacagagggcagaaccagcgatatgagaacagaaatt
atccgtatgatggagtccgcaaaacctgaggacgtgtccttccaaggc
agaggcgtgttcgagctgagcgacgagaaggccaccaaccctatcgtg
cctagcttcgatatgtctaatgagggcagctactttttcggagataac
gccgaagagtacgacaacatgtctaatatggatatcgacggcattaac
accggcaccatcgacaaaacccctgaggagatcacccctggcaccagc
ggcacaacccggcccatcatccgccccgctacactggctccacctagc
aacaagcggaccagaaatccctcgccagaaagagccacaacctccagc
gaggacgacgtgggacggaagacacaaaagaagcagacccctacagag
atcaagaagtctgtttacaacatggtggtgaaactgggcgagttctac
aaccagatgatggtgaaggccggcctgaacgacgatatggaaagaaat
ctgatccagaacgcccacgccgtggagcggattctgctggccgccacc
gatgataagaagaccgaattccagaaaaagaaaaacgccagagacgtg
aaggaaggcaaggaagagatcgaccacaacaagacaggcggcacattc
tacaagatggtccgggacgacaagaccatctacttcagccctatccgg
ataacattcctgaaagaagaagtgaagaccatgtacaaaaccacaatg
ggctctgacggcttcagcggcctgaatcacatcatgatcggccactct
caaatgaacgatgtgtgcttccagagaagcaaggctctgaagcgcgtg
ggcctggatcctagcctgatctctaccttcgccggcagcaccatcccc
agaagatcgggcgctaccggcgtggctatcaagggaggaggcacactg
gtggctgaagccatcagattcatcggaagagccatggccgacagagga
ctcctgagagatatcaaagccaaaaccgcctacgaaaaaatcctgctg
aacctgaagaacaagtgcagcgcgcctcaacagaaggccctggtggac
caggttatcggctctagaaaccctggaatcgccgatatcgaggacctg
acactgctggccagatctatggtggtggtgagaccctccgtggccagc
aaggtggtgctgcctatcagcatctacgccaagatccctcagctggga
tttaacgtggaagaatacagcatggttggttatgaggccatggccctg
tacaacatggccacacctgtgtccatcctgagaatgggcgacgatgcc
aaagacaagagccagctgttcttcatgagctgcttcggcgctgcctat
gaggacctgagagtgctgtccgctcttacaggaacagagttcaagcct
aggagcgcactgaagtgcaagggcttccacgtgcccgccaaggaacag
gtggaaggcatgggagctgctctgatgtccatcaagctgcaattttgg
gctcctatgacccggagcggcggaaatgaggtgggtggcgacggaggc
agcggacagatttcttgcagccccgtatttgccgtggagagaccaatc
gccctgtccaagcaggccgtgagaagaatgctgagcatgaacatcgag
ggccgggacgccgacgtgaagggcaacctgttgaagatgatgaacgac
agcatggccaagaagaccagtggcaatgccttcatcggcaagaagatg
ttccagatctccgacaagaacaagaccaaccccgtggaaatccccatc
aagcagacaatccctaacttcttcttcggcagagacaccgccgaagac
tatgacgacctggactacataggaaattgcccaatatgggtgaaaaca
cctttgaagcttgccaatggaaccaaatatagacctcctgcaaaacta
ttaaaggaaaggggtttcttcggagctattgctggtttcctagaagga
ggatgggaaggaatgattgcaggctggcacggatacacatctcacgga
gcacatggagtggcagtggcggcggaccttaagagtacgcaagaagct
ataaacaagataacaaaaaatctcaattctttgagtgagctagaagta
aagaatcttcaaagactaagtggtgccatggatgaactccacaacgaa
atactcgagctggatgagaaagtggatgatctcagagctgacactata
agctcgcaaatagaacttgcagtcttgctttccaacgaaggaataata
aacagtgaagatgagtga
>G50012 (synthetic protein)
SEQ ID No: 29
MPMGSLQPLATLYLLGMLVASCLGIGECPKYVRSAKLRMVTGLRNIPS
IQSRGLFGAIAGFIEGGWTGMVDGWYGYHHQNEQGSGYAADQKSTQNA
INGITNKVNSVIEKMNTQFTAVGKEENKLERRMENLNKKVDDGFLDIW
TYNAELLVLLENERTLDFHDSASQGTKRSYEQMETGGERQNATEIRAS
VGRMVGGIGRFYIQMCTELKLSDYEGRLIQNSITIERMVLSAFDERRN
KYLEEHPSAGKDPKKTGGPIYRRRDGKWVRELILYDKEEIRRIWRQAN
NGEDATAGLTHLMIWHSNLNDATYQRTRALVRTGMDPRMCSLMQGSTL
PRRSGAAGAAVKGVGTMVMELIRMIKRGINDRNFWRGENGRRTRIAYE
RMCNILKGKFQTAAQRAMMDQVRESRNPGNAEIEDLIFLARSALILRG
SVAHKSCLPACVYGLAVASGYDFEREGYSLVGIDPFRLLQNSQVFSLI
RPNENPAHKSQLVWMACHSAAFEDLRVSSFIRGTRVVPRGQLSTRGVQ
IASNENMETMDSSTLELRSRYWAIRTRSGGTTNQQRASAGQISVQPTF
SVQRNLPFERATIMAAFTGNTEGRTSDMRTEIIRMMESAKPEDVSFQG
RGVFELSDEKATNPIVPSFDMSNEGSYFFGDNAEEYDNMSNMDIDGIN
TGTIDKTPEEITPGTSGTTRPIIRPATLAPPSNKRTRNPSPERATTSS
EDDVGRKTQKKQTPTEIKKSVYNMVVKLGEFYNQMMVKAGLNDDMERN
LIQNAHAVERILLAATDDKKTEFQKKKNARDVKEGKEEIDHNKTGGTF
YKMVRDDKTIYFSPIRITFLKEEVKTMYKTTMGSDGFSGLNHIMIGHS
QMNDVCFQRSKALKRVGLDPSLISTFAGSTIPRRSGATGVAIKGGGTL
VAEAIRFIGRAMADRGLLRDIKAKTAYEKILLNLKNKCSAPQQKALVD
QVIGSRNPGIADIEDLTLLARSMVVVRPSVASKVVLPISIYAKIPQLG
ENVEEYSMVGYEAMALYNMATPVSILRMGDDAKDKSQLFEMSCFGAAY
EDLRVLSALTGTEFKPRSALKCKGFHVPAKEQVEGMGAALMSIKLQFW
APMTRSGGNEVGGDGGSGQISCSPVFAVERPIALSKQAVRRMLSMNIE
GRDADVKGNLLKMMNDSMAKKTSGNAFIGKKMFQISDKNKTNPVEIPI
KQTIPNFFFGRDTAEDYDDLDYIGNCPIWVKTPLKLANGTKYRPPAKL
LKERGFFGAIAGFLEGGWEGMIAGWHGYTSHGAHGVAVAADLKSTQEA
INKITKNLNSLSELEVKNLQRLSGAMDELHNEILELDEKVDDLRADTI
SSQIELAVLLSNEGIINSEDE

What is claimed:

1. A composition for stimulating an immune response against a coronavirus in a mammalian subject, comprising an excipient, and a messenger RNA (mRNA) comprising an open reading frame (ORF) encoding a fusion protein, wherein the ORF comprises from 5′ to 3′:
(i) a nucleotide sequence encoding a mammalian signal peptide; and
(ii) a nucleotide sequence encoding a coronavirus nucleocapsid protein.

2. The composition of claim 1, wherein the coronavirus is a betacoronavirus, optionally wherein the betacoronavirus is a human betacoronavirus.

3. The composition of claim 2, wherein the betacoronavirus comprises a severe acute respiratory syndrome coronavirus-2 (SARS-COV-2), a severe acute respiratory syndrome coronavirus-1 (SARS-COV-1), a middle east respiratory syndrome-related coronavirus (MERS-CoV), or a combination thereof.

4. The composition of claim 3, wherein the betacoronavirus comprises a severe acute respiratory syndrome coronavirus-2 (SARS-COV-2).

5. The composition of claim 4, wherein the coronavirus nucleocapsid protein comprises a first nucleocapsid protein and a second nucleocapsid protein, wherein the first nucleocapsid protein is a SARS-COV-2 nucleocapsid protein of a first variant from a first clade, and the second nucleocapsid protein is a SARS-COV-2 nucleocapsid protein of a second variant from a second clade, and wherein the first clade and the second clade are different clades as defined by one or more of the World Health Organization, Pango, GISAID, and Nextstrain.

6. A composition for stimulating an immune response against a coronavirus in a mammalian subject, comprising an excipient, and a messenger RNA (mRNA) comprising an open reading frame (ORF) encoding a fusion protein, wherein the ORF comprises from 5′ to 3′:
(i) a nucleotide sequence encoding a mammalian signal peptide; and
(ii) a nucleotide sequence encoding two or more coronavirus nucleocapsid proteins.

7. The composition of claim 6, wherein the coronavirus is a betacoronavirus, optionally wherein the betacoronavirus is a human betacoronavirus.

8. The composition of claim 7, wherein the betacoronavirus comprises a severe acute respiratory syndrome coronavirus-2 (SARS-COV-2), a severe acute respiratory syndrome coronavirus-1 (SARS-COV-1), a middle east respiratory syndrome-related coronavirus (MERS-CoV), or a combination thereof.

9. The composition of claim 8, wherein the betacoronavirus comprises a severe acute respiratory syndrome coronavirus-2 (SARS-COV-2).

10. The composition of claim 9, wherein the two or more coronavirus nucleocapsid proteins comprise a SARS-COV-2 nucleocapsid protein and a MERS nucleocapsid protein.

11. The composition of claim 9, wherein the two or more coronavirus nucleocapsid proteins comprise a SARS-COV-2 nucleocapsid protein, a SARS-COV-1 nucleocapsid protein, and a MERS nucleocapsid protein.

12. The composition of any one of claims 6-11, wherein the two or more coronavirus nucleocapsid proteins are separated by a linker of from one to ten residues in length.

13. The composition of any one of claims 1-12, wherein the mammalian signal peptide is a signal peptide of a surface protein expressed in mammalian antigen presenting cells.

14. The composition of claim 13, wherein the mammalian signal peptide is a CD5 signal peptide and the amino acid sequence of the CD5 signal peptide comprises SEQ ID NO:8, or the amino acid sequence at least 90% or 95% identical to SEQ ID NO:8.

15. The composition of any one of claims 1-14, wherein the amino acid sequence of the nucleocapsid protein comprises residues 2-419 of SEQ ID NO:5, or the amino acid sequence at least 75%, 85%, 90%, 95%, 96%, 97%, 98% or 99% identical to residues 2-419 of SEQ ID NO:5.

16. The composition of any one of claims 1-14, wherein the amino acid sequence of the fusion protein comprises SEQ ID NO:6, or the amino acid sequence at least 75%, 85%, 90%, 95%, 96%, 97%, 98% or 99% identical to SEQ ID NO:6.

17. The composition of any one of claims 6-14, wherein the amino acid sequence of the fusion protein comprises SEQ ID NO:7, or the amino acid sequence at least 75%, 85%, 90%, 95%, 96%, 97%, 98% or 99% identical to SEQ ID NO:7.

18. The composition of claim 16, wherein the open reading frame comprises the nucleotide sequence of SEQ ID NO:2.

19. The composition of claim 17, wherein the open reading frame comprises the nucleotide sequence of SEQ ID NO:3 or SEQ ID NO:4.

20. The composition of any one of claims 1-14, wherein the amino acid sequence of the fusion protein comprises residues 2-413 of SEQ ID NO:9, or the amino acid sequence at least 75%, 85%, 90%, 95%, 96%, 97%, 98% or 99% identical to residues 2-413 of SEQ ID NO:9.

21. The composition of any one of claims 1-14, wherein the amino acid sequence of the fusion protein comprises residues 2-422 of SEQ ID NO:10, or the amino acid sequence at least 75%, 85%, 90%, 95%, 96%, 97%, 98% or 99% identical to residues 2-422 of SEQ ID NO:10.

22. The composition of any one of claims 1-21, wherein the composition does not comprise liposomes or lipid nanoparticles.

23. The composition of any one of claims 1-22, wherein the mRNA is a self-replicating mRNA.

24. The composition of claim 23, wherein the self-replicating RNA comprises an Alphavirus replicon lacking a viral structural protein coding region.

25. The composition of claim 24, wherein the Alphavirus is selected from the group consisting of a Venezuelan equine encephalitis virus, a Sindbis virus, and a Semliki Forrest virus. 26 The composition of claim 25, wherein the Alphavirus is a Venezuelan equine encephalitis virus.

27. The composition of any one of claims 23-26, wherein the Alphavirus replicon comprises a nonstructural protein coding region with an insertion of 12-18 nucleotides resulting in expression of a nonstructural Protein 2 (nsP2) comprising from 4 to 6 additional amino acids between beta sheet 4 and beta sheet 6 of the nsP2.

28. The composition of any one of claims 1-27, wherein the self-replicating mRNA is a temperature-sensitive agent (ts-agent) that is capable of expressing the fusion at a permissive temperature but not at a non-permissive temperature.

29. The composition of claim 28, wherein the permissive temperature is from 31° C. to 35° C. and the non-permissive temperature is at least 37° C.±0.5° C.

30. A method for stimulating an immune response against a coronavirus in a mammalian subject, comprising administering the composition of any one of claims 1-29 to a mammalian subject so as to stimulate an immune response against the coronavirus nucleocapsid protein in the mammalian subject

31. The method of claim 30, wherein the composition is administered intradermally.

32. The method of claim 30 or claim 31, wherein the immune response comprises a coronavirus-reactive cellular immune response.

33. The method of claim 32, wherein the immune response further comprises a coronavirus-reactive humoral immune response.

34. The method of any one of claims 30-33, wherein the mammalian subject is a human subject.

35. A kit comprising:
the composition of any one of claims 1-29 or any one of claims 37-62; and
a device for intradermal delivery of the composition to a mammalian subject.

36. The kit of claim 35, wherein the device comprises a syringe and a needle.

37. A composition for stimulating an immune response against two or more viruses in a mammalian subject, comprising an excipient, and a messenger RNA (mRNA) comprising an open reading frame (ORF) encoding a fusion protein, wherein the ORF comprises from 5′ to 3′:
(i) a nucleotide sequence encoding a mammalian signal peptide; and
(ii) a nucleotide sequence encoding a first nucleocapsid protein of a first virus and a second nucleocapsid protein of a second virus.

38. The composition of claim 37, wherein the first and second viruses are capable of causing disease upon infection of a human subject.

39. The composition of claim 38, wherein the first and second viruses are different variants, subtypes or lineages of the same species.

40. The composition of claim 38, wherein the first and second viruses are different species of the same genus.

41. The composition of claim 40, wherein the first and second viruses are both members of the betacoronavirus genus.

42. The composition of claim 41, wherein the first and second viruses comprise a severe acute respiratory syndrome coronavirus-2 (SARS-COV-2) and a middle east respiratory syndrome-related coronavirus (MERS-COV).

43. The composition of claim 38, wherein the first and second viruses are members of different families, orders, classes, or phyla of the same kingdom.

44. The composition of claim 43, wherein the first and second viruses are both members of the orthomyxoviridae family.

45. The composition of claim 44, wherein the first and second viruses comprise an influenza A virus and an influenza B virus.

46. The composition of claim 45, wherein the amino acid sequence of the fusion protein comprises SEQ ID NO:16, or the amino acid sequence at least 75%, 85%, 90%, 95%, 96%, 97%, 98% or 99% identical to SEQ ID NO:16.

47. The composition of claim 38, wherein the first and second viruses are both members of the orthornavirae kingdom, optionally wherein the first and second viruses comprise: (a) a severe acute respiratory syndrome coronavirus-2 (SARS-COV-2), a severe acute respiratory syndrome coronavirus-1 (SARS-COV-1), or a middle east respiratory syndrome-related coronavirus (MERS-COV); and (b) an influenza A virus or an influenza B virus.

48. The composition of claim 40, wherein the first and second viruses are both members of the ebolavirus genus, optionally wherein the first and second viruses are selected from the group consisting of Zaire ebolavirus, Sudan ebolavirus, Bundibugyo ebolavirus, and Taï Forest ebolavirus.

49. The composition of claim 48, wherein the nucleotide sequence further encodes a third nucleocapsid protein of a third virus and a fourth nucleocapsid protein of a fourth virus, and the first, second, third and fourth viruses are Zaire ebolavirus, Sudan ebolavirus, Bundibugyo ebolavirus, and Taï Forest ebolavirus.

50. The composition of claim 49, wherein the amino acid sequence of the fusion protein comprises SEQ ID NO:22, or the amino acid sequence at least 75%, 85%, 90%, 95%, 96%, 97%, 98% or 99% identical to SEQ ID NO:22.

51. The composition of claim 49, wherein the nucleotide sequence (ii) encodes a shared portion of the first nucleocapsid protein of the first virus for stimulating an immune response against all of the first, second, third and fourth viruses.

52. The composition of claim 51, wherein the nucleotide sequence (ii) encodes an individual portion of each of the first, second, third and fourth nucleocapsid proteins for stimulating an immune response against all of the first, second, third and fourth viruses.

53. The composition of claim 52, wherein the nucleotide sequence (ii) encodes a fragment of the individual portion of the second nucleocapsid protein of the second virus for stimulating an immune response against the second and third viruses.

54. The composition of claim 37, wherein the nucleotide sequence (ii) encodes a shared portion of the first nucleocapsid protein of the first virus for stimulating an immune response against both the first and second viruses.

55. The composition of claim 54, wherein the nucleotide sequence (ii) encodes an individual portion of each of the first and second nucleocapsid proteins for stimulating an immune response against both the first and second viruses.

56. The composition of any one of claims 37-48, wherein the nucleotide sequence of (ii) further encodes at least one further nucleocapsid protein of at least one further virus, and wherein the at least one further virus is different from the first and second viruses.

57. The composition of any one of claims 37-56, wherein the first and second, or the first, second, and further nucleocapsid proteins are separated by a linker of from one to ten residues in length.

58. The composition of any one of claims 37-57, wherein the mammalian signal peptide is a signal peptide of a surface protein expressed in mammalian antigen presenting cells.

59. The composition of any one of claims 37-58, wherein the mRNA is a self-replicating mRNA.

60. The composition of claim 59, wherein the self-replicating mRNA is a temperature-sensitive agent (ts-agent) that is capable of expressing the fusion protein a permissive temperature but not at a non-permissive temperature.

61. The composition of claim 60, wherein the permissive temperature is from 31° C. to 35° C. and the non-permissive temperature is at least 37° C.±0.5° ° C.

62. The composition of any one of claims 1-29 or any one of claims 37-61, wherein the composition further comprises chitosan.

63. A method for stimulating an immune response against two or more viruses in a mammalian subject, comprising administering the composition of any one of claims 37-62 to a mammalian subject to stimulate an immune response against the nucleocapsid proteins of the two or more viruses in the mammalian subject

64. The method of claim 63, wherein the composition is administered intradermally.

65. The method of claim 63 or claim 64, wherein the immune response comprises a cellular immune response reactive with the two or more viruses.

66. The method of claim 65, wherein the cellular immune response comprises a nucleocapsid protein-specific helper T lymphocyte (Th) response comprising nucleocapsid protein-specific cytokine secretion.

67. The method of claim 66, wherein nucleocapsid protein-specific cytokine secretion comprises secretion of one or both of interferon-gamma and interleukin-4.

68. The method of claim 65, wherein the cellular immune response comprises a nucleocapsid protein-specific cytotoxic T lymphocyte (CTL) response.

69. The method of any one of claims 65-68, wherein the immune response further comprises a humoral immune response reactive with the two or more viruses.

70. The method of any one of claims 63-69, wherein the mammalian subject is a human subject.

71. A composition for stimulating an immune response against a virus in a mammalian subject, comprising an excipient, and a messenger RNA (mRNA) comprising an open reading frame (ORF) encoding a fusion protein, wherein the ORF comprises from 5′ to 3′:
(i) a nucleotide sequence encoding a mammalian signal peptide;
(ii) a nucleotide sequence encoding a first viral antigen or fragment thereof of a first virus; and
(iii) a nucleotide sequence encoding a second viral antigen or fragment thereof of the first virus or a second virus,
wherein the first viral antigen is a nucleocapsid protein and the second viral antigen is a surface protein, or the first viral antigen is a surface protein and the second viral antigen is a nucleocapsid protein.

72. A composition for stimulating an immune response against two or more viruses in a mammalian subject, comprising an excipient, and a messenger RNA (mRNA) comprising an open reading frame (ORF) encoding a fusion protein, wherein the ORF comprises from 5′ to 3′:
(i) a nucleotide sequence encoding a mammalian signal peptide;
(ii) a nucleotide sequence encoding a first viral antigen or fragment thereof of a first virus;
(iii) a nucleotide sequence encoding a second viral antigen or fragment thereof of the first virus;
(iv) a nucleotide sequence encoding a third viral antigen or fragment thereof of a second virus;
(iii) a nucleotide sequence encoding a fourth viral antigen or fragment thereof of the second virus,
wherein the first viral antigen is a first nucleocapsid protein and the second viral antigen is a first surface protein, or the first viral antigen is a first surface protein and the second viral antigen is a first nucleocapsid protein, and
wherein the third viral antigen is a second nucleocapsid protein and the fourth viral antigen is a second surface protein, or the third viral antigen is a second surface protein and the fourth viral antigen is a second nucleocapsid protein.

73. The composition of claim 71 or claim 72, wherein the mRNA is a self-replicating mRNA.

74. The composition of claim 73, wherein the self-replicating RNA comprises an Alphavirus replicon lacking a viral structural protein coding region.

75. The composition of claim 74, wherein the Alphavirus is selected from the group consisting of a Venezuelan equine encephalitis virus, a Sindbis virus, and a Semliki Forrest virus.

76. The composition of claim 74, wherein the Alphavirus is a Venezuelan equine encephalitis virus.

77. The composition of any one of claims 73-76, wherein the self-replicating mRNA is a temperature-sensitive agent (ts-agent) that is capable of expressing the fusion protein at a permissive temperature but not at a non-permissive temperature.

78. The composition of claim 77, wherein the permissive temperature is from 31° C. to 35° C., and the non-permissive temperature is at least 37° C.±0.5° ° C.

79. The composition of any one of claims 74-78, wherein the Alphavirus replicon comprises a nonstructural protein coding region with an insertion of 12-18 nucleotides resulting in expression of a nonstructural Protein 2 (nsP2) comprising from 4 to 6 additional amino acids between beta sheet 4 and beta sheet 6 of the nsP2.

80. The composition of any one of claims 71-79, wherein the first virus and/or the second virus is a coronavirus, optionally wherein the coronavirus is a betacoronavirus, optionally wherein the betacoronavirus is a human betacoronavirus.

81. The composition of claim 80, wherein the first and/or the second virus is a betacoronavirus independently selected from the group consisting of a severe acute respiratory syndrome coronavirus-2 (SARS-COV-2), a severe acute respiratory syndrome coronavirus-1 (SARS-COV-1), and a middle east respiratory syndrome-related coronavirus (MERS-COV).

82. The composition of claim 80, wherein the first virus is SARS-COV-2 and the second virus is MERS-COV.

83. The composition of any one of claims 80-82, wherein the surface protein, the first surface protein and/or the second surface protein each comprise a receptor-binding domain (RBD) of a coronavirus Spike protein.

84. The composition of claim 83, wherein the amino acid sequence of the fusion protein comprises SEQ ID NO:27, or the amino acid sequence at least 75%, 85%, 90%, 95%, 96%, 97%, 98% or 99% identical to SEQ ID NO:27.

85. The composition of any one of claims 71-79, wherein the first virus and/or the second virus is a member of the orthomyxoviridae family.

86. The composition of claim 85, wherein the first and/or the second virus is independently selected from the group consisting of an influenza A virus (IAV) and an influenza B virus (IBV).

87. The composition of claim 86, wherein the first virus is IAV and the second virus is IBV.

88. The composition of any one of claims 85-87, wherein the surface protein, the first surface protein and/or the second surface protein each comprise a portion of an influenza hemagglutinin.

89. The composition of claim 88, wherein the amino acid sequence of the fusion protein comprises SEQ ID NO:29, or the amino acid sequence at least 75%, 85%, 90%, 95%, 96%, 97%, 98% or 99% identical to SEQ ID NO:29.

90. The composition of any one of claims 71-89, wherein the composition further comprises chitosan.

91. A kit comprising:
(i) the composition of any one of claims 71-90; and
(ii) a device for intradermal delivery of the composition to a mammalian subject.

92. The kit of claim 91, wherein the device comprises a syringe and a needle.

93. The kit of claim 91 or claim 92, further comprising instructions for use of the device to administer the composition to a mammalian subject to stimulate an immune response against one or more of the first viral antigen, the second viral antigen, the third viral antigen, and the fourth viral antigen.

94. A method of stimulating an immune response in a mammalian subject, comprising administering the composition of any one of claims 71-90 to a mammalian subject to stimulate an immune response against one or more of the first viral antigen, the second viral antigen, the third viral antigen, and the fourth viral antigen in the mammalian subject.

95. The method of claim 94, wherein the composition is administered intradermally.

96. The method of claim 95, wherein the immune response comprises a cellular immune response reactive against one or more of the first viral antigen, the second viral antigen, the third viral antigen, and the fourth viral antigen.

97. The method of claim 96, wherein the immune response further comprises a humoral immune response reactive against one or more of the first viral antigen, the second viral antigen, the third viral antigen, and the fourth viral antigen.

98. The method of any one of claims 94-97, wherein the mammalian subject is a human subject.

99. A method for active booster immunization against at least one virus, comprising intradermally administering the composition of any one of claims 1-29, any one of claims 37-62, or any one of claims 71-90 to a mammalian subject in need thereof to stimulate a secondary immune response against the virus, wherein the mammalian subject had already undergone a primary immunization regimen against the virus.

100. The method of claim 99, wherein the primary immunization regimen comprises administration of at least one dose of a different vaccine against the virus.

101. The method of claim 100, wherein the different vaccine comprises a protein antigen of the at least one virus, optionally wherein the protein antigen is a recombinant protein or fragment thereof, or an inactivated virus.

102. A method for active booster immunization against at least one virus, comprising:
(i) intradermally administering the composition of any one of claims 1-29, any one of claims 37-62, or any one of claims 71-90 to a mammalian subject in need thereof to stimulate a primary immune response against the virus; and
(ii) administering at least one dose of a different vaccine against the virus to the mammalian subject to stimulate a secondary immune response against the virus.

103. The method of claim 102, wherein the different vaccine comprises a protein antigen of the at least one virus, optionally wherein the protein antigen is a recombinant protein or fragment thereof, or an inactivated virus.

104. A method for active primary immunization against at least one virus, comprising:
(i) intradermally administering the composition of any one of claims 1-29, any one of claims 37-62, or any one of claims 71-90 to a mammalian subject in need thereof to stimulate a primary immune response against the virus; wherein the mammalian subject had not undergone a primary immunization regimen against the virus.

105. The method of claim 104, further comprising:
(ii) administering at least one dose of a different vaccine against the virus to the mammalian subject to stimulate a secondary immune response against the virus.

106. The method of claim 105, wherein the different vaccine comprises a protein antigen of the at least one virus, optionally wherein the protein antigen is a recombinant protein or fragment thereof, or an inactivated virus.

107. The method of any one of claims 94-106, wherein the mammalian subject is a human subject.
